SEQUENCE LISTING (627 sequences)

SEQ ID NO: 1 - DNA, 20 bp, artificial
  human mitoNEET antisense
ttgtctccagtctcttcgtt

SEQ ID NO: 2 - DNA, 20 bp, artificial
  human mitoNEET antisense
gtctccagtctcttcgttat

SEQ ID NO: 3 - DNA, 20 bp, artificial
  human mitoNEET antisense
tgtctccagtctcttcgtta

SEQ ID NO: 4 - DNA, 20 bp, artificial
  human mitoNEET antisense
attgtctccagtctcttcgt

SEQ ID NO: 5 - DNA, 20 bp, artificial
  human mitoNEET antisense
ctccagtctcttcgttatgt

SEQ ID NO: 6 - DNA, 20 bp, artificial
  human mitoNEET antisense
tccagtctcttcgttatgtt

SEQ ID NO: 7 - DNA, 20 bp, artificial
  human mitoNEET antisense
tctccagtctcttcgttatg

SEQ ID NO: 8 - DNA, 20 bp, artificial
  human mitoNEET antisense
ccagtctcttcgttatgttt

SEQ ID NO: 9 - DNA, 20 bp, artificial
  human mitoNEET antisense
gtctcttcgttatgttttgt

SEQ ID NO: 10 - DNA, 20 bp, artificial
  human mitoNEET antisense
cattgtctccagtctcttcg

SEQ ID NO: 11 - DNA, 20 bp, artificial
  human mitoNEET antisense
accgagctcaaacgggttcg

SEQ ID NO: 12 - DNA, 20 bp, artificial
  human mitoNEET antisense
cagtctcttcgttatgtttt

SEQ ID NO: 13 - DNA, 20 bp, artificial
  human mitoNEET antisense
ccgagctcaaacgggttcgc

SEQ ID NO: 14 - DNA, 20 bp, artificial
  human mitoNEET antisense
agtctcttcgttatgttttg

SEQ ID NO: 15 - DNA, 20 bp, artificial
  human mitoNEET antisense
gataccgagctcaaacgggt

SEQ ID NO: 16 - DNA, 20 bp, artificial
  human mitoNEET antisense
taccgagctcaaacgggttc

SEQ ID NO: 17 - DNA, 20 bp, artificial
  human mitoNEET antisense
ataccgagctcaaacgggtt

SEQ ID NO: 18 - DNA, 20 bp, artificial
  human mitoNEET antisense
acattgtctccagtctcttc

SEQ ID NO: 19 - DNA, 20 bp, artificial
  human mitoNEET antisense
ggataccgagctcaaacggg

SEQ ID NO: 20 - DNA, 20 bp, artificial
  human mitoNEET antisense
gagctcaaacgggttcgcgc

SEQ ID NO: 21 - DNA, 20 bp, artificial
  human mitoNEET antisense
tctcttcgttatgttttgtg

SEQ ID NO: 22 - DNA, 20 bp, artificial
  human mitoNEET antisense
ttagaatccagcgaaggtga

SEQ ID NO: 23 - DNA, 20 bp, artificial
  human mitoNEET antisense
agctcaaacgggttcgcgcg

SEQ ID NO: 24 - DNA, 20 bp, artificial
  human mitoNEET antisense
cacattgtctccagtctctt

SEQ ID NO: 25 - DNA, 20 bp, artificial
  human mitoNEET antisense
atcctccatgtcaaaagcat

SEQ ID NO: 26 - DNA, 20 bp, artificial
  human mitoNEET antisense
ccacattgtctccagtctct

SEQ ID NO: 27 - DNA, 20 bp, artificial
  human mitoNEET antisense
ttcaactcgtacgctggaac

SEQ ID NO: 28 - DNA, 20 bp, artificial
  human mitoNEET antisense
agaatccagcgaaggtgaat

SEQ ID NO: 29 - DNA, 20 bp, artificial
  human mitoNEET antisense
ctcttcgttatgttttgtgt

SEQ ID NO: 30 - DNA, 20 bp, artificial
  human mitoNEET antisense
cctccatgtcaaaagcatgt

SEQ ID NO: 31 - DNA, 20 bp, artificial
  human mitoNEET antisense
tagaatccagcgaaggtgaa

SEQ ID NO: 32 - DNA, 20 bp, artificial
  human mitoNEET antisense
tcctccatgtcaaaagcatg

SEQ ID NO: 33 - DNA, 20 bp, artificial
  human mitoNEET antisense
cgagctcaaacgggttcgcg

SEQ ID NO: 34 - DNA, 20 bp, artificial
  human mitoNEET antisense
tgcaccacgatgtttcaaca

SEQ ID NO: 35 - DNA, 20 bp, artificial
  human mitoNEET antisense
ctaggataccgagctcaaac

SEQ ID NO: 36 - DNA, 20 bp, artificial
  human mitoNEET antisense
actaggataccgagctcaaa

SEQ ID NO: 37 - DNA, 20 bp, artificial
  human mitoNEET antisense
tcaactcgtacgctggaact

SEQ ID NO: 38 - DNA, 20 bp, artificial
  human mitoNEET antisense
actcgtacgctggaactgga

SEQ ID NO: 39 - DNA, 20 bp, artificial
  human mitoNEET antisense
aatcctccatgtcaaaagca

SEQ ID NO: 40 - DNA, 20 bp, artificial
  human mitoNEET antisense
tttagaatccagcgaaggtg

SEQ ID NO: 41 - DNA, 20 bp, artificial
  human mitoNEET antisense
aatccagcgaaggtgaatca

SEQ ID NO: 42 - DNA, 20 bp, artificial
  human mitoNEET antisense
tccattcaactcgtacgctg

SEQ ID NO: 43 - DNA, 20 bp, artificial
  human mitoNEET antisense
cagcgaaggtgaatcagaca

SEQ ID NO: 44 - DNA, 20 bp, artificial
  human mitoNEET antisense
gtgaatcagacagaggtggt

SEQ ID NO: 45 - DNA, 20 bp, artificial
  human mitoNEET antisense
gcgaaggtgaatcagacaga

SEQ ID NO: 46 - DNA, 20 bp, artificial
  human mitoNEET antisense
attcaactcgtacgctggaa

SEQ ID NO: 47 - DNA, 20 bp, artificial
  human mitoNEET antisense
cccacattgtctccagtctc

SEQ ID NO: 48 - DNA, 20 bp, artificial
  human mitoNEET antisense
gaatccagcgaaggtgaatc

SEQ ID NO: 49 - DNA, 20 bp, artificial
  human mitoNEET antisense
gcaccacgatgtttcaacaa

SEQ ID NO: 50 - DNA, 20 bp, artificial
  human mitoNEET antisense
ctcgtacgctggaactggaa

SEQ ID NO: 51 - DNA, 20 bp, artificial
  human mitoNEET antisense
ggtgaatcagacagaggtgg

SEQ ID NO: 52 - DNA, 20 bp, artificial
  human mitoNEET antisense
gctcaaacgggttcgcgcgg

SEQ ID NO: 53 - DNA, 20 bp, artificial
  human mitoNEET antisense
aggataccgagctcaaacgg

SEQ ID NO: 54 - DNA, 20 bp, artificial
  human mitoNEET antisense
atccagcgaaggtgaatcag

SEQ ID NO: 55 - DNA, 20 bp, artificial
  human mitoNEET antisense
atccattcaactcgtacgct

SEQ ID NO: 56 - DNA, 20 bp, artificial
  human mitoNEET antisense
agcgaaggtgaatcagacag

SEQ ID NO: 57 - DNA, 20 bp, artificial
  human mitoNEET antisense
atttcgatgatctttaacat

SEQ ID NO: 58 - DNA, 20 bp, artificial
  human mitoNEET antisense
ccacatttagaatccagcga

SEQ ID NO: 59 - DNA, 20 bp, artificial
  human mitoNEET antisense
ctcccaaatcctccatgtca

SEQ ID NO: 60 - DNA, 20 bp, artificial
  human mitoNEET antisense
aatgtgcaccacgatgtttc

SEQ ID NO: 61 - DNA, 20 bp, artificial
  human mitoNEET antisense
tttcgatgatctttaacata

SEQ ID NO: 62 - DNA, 20 bp, artificial
  human mitoNEET antisense
tatttcgatgatctttaaca

SEQ ID NO: 63 - DNA, 20 bp, artificial
  human mitoNEET antisense
cattcaactcgtacgctgga

SEQ ID NO: 64 - DNA, 20 bp, artificial
  human mitoNEET antisense
ctccatgtcaaaagcatgta

SEQ ID NO: 65 - DNA, 20 bp, artificial
  human mitoNEET antisense
accacatttagaatccagcg

SEQ ID NO: 66 - DNA, 20 bp, artificial
  human mitoNEET antisense
cctccaacaacggcagtaca

SEQ ID NO: 67 - DNA, 20 bp, artificial
  human mitoNEET antisense
atttagaatccagcgaaggt

SEQ ID NO: 68 - DNA, 20 bp, artificial
  human mitoNEET antisense
tcgtacgctggaactggaag

SEQ ID NO: 69 - DNA, 20 bp, artificial
  human mitoNEET antisense
ccatgtcaaaagcatgtact

SEQ ID NO: 70 - DNA, 20 bp, artificial
  human mitoNEET antisense
tgaatcagacagaggtggta

SEQ ID NO: 71 - DNA, 20 bp, artificial
  human mitoNEET antisense
aaatcctccatgtcaaaagc

SEQ ID NO: 72 - DNA, 20 bp, artificial
  human mitoNEET antisense
tcccaaatcctccatgtcaa

SEQ ID NO: 73 - DNA, 20 bp, artificial
  human mitoNEET antisense
tccagcgaaggtgaatcaga

SEQ ID NO: 74 - DNA, 20 bp, artificial
  human mitoNEET antisense
cggcgagagtaaaggtgcca

SEQ ID NO: 75 - DNA, 20 bp, artificial
  human mitoNEET antisense
gcccacattgtctccagtct

SEQ ID NO: 76 - DNA, 20 bp, artificial
  human mitoNEET antisense
aggtgaatcagacagaggtg

SEQ ID NO: 77 - DNA, 20 bp, artificial
  human mitoNEET antisense
atgtgcaccacgatgtttca

SEQ ID NO: 78 - DNA, 20 bp, artificial
  human mitoNEET antisense
taggataccgagctcaaacg

SEQ ID NO: 79 - DNA, 20 bp, artificial
  human mitoNEET antisense
ttcgatgatctttaacataa

SEQ ID NO: 80 - DNA, 20 bp, artificial
  human mitoNEET antisense
tccatgtcaaaagcatgtac

SEQ ID NO: 81 - DNA, 20 bp, artificial
  human mitoNEET antisense
cgaaggtgaatcagacagag

SEQ ID NO: 82 - DNA, 20 bp, artificial
  human mitoNEET antisense
cactaggataccgagctcaa

SEQ ID NO: 83 - DNA, 20 bp, artificial
  human mitoNEET antisense
caactcgtacgctggaactg

SEQ ID NO: 84 - DNA, 20 bp, artificial
  human mitoNEET antisense
tcttcgttatgttttgtgtg

SEQ ID NO: 85 - DNA, 20 bp, artificial
  human mitoNEET antisense
aaatgtgcaccacgatgttt

SEQ ID NO: 86 - DNA, 20 bp, artificial
  human mitoNEET antisense
tcagacagaggtggtagtca

SEQ ID NO: 87 - DNA, 20 bp, artificial
  human mitoNEET antisense
ttttttttttttttttgttt

SEQ ID NO: 88 - DNA, 20 bp, artificial
  human mitoNEET antisense
ccggcgagagtaaaggtgcc

SEQ ID NO: 89 - DNA, 20 bp, artificial
  human mitoNEET antisense
gccgtcgcttgcaaaggcgt

SEQ ID NO: 90 - DNA, 20 bp, artificial
  human mitoNEET antisense
cgtacgctggaactggaagt

SEQ ID NO: 91 - DNA, 20 bp, artificial
  human mitoNEET antisense
gtcaaaagcatgtactatct

SEQ ID NO: 92 - DNA, 20 bp, artificial
  human mitoNEET antisense
taccacatttagaatccagc

SEQ ID NO: 93 - DNA, 20 bp, artificial
  human mitoNEET antisense
gcactaggataccgagctca

SEQ ID NO: 94 - DNA, 20 bp, artificial
  human mitoNEET antisense
atgtcaaaagcatgtactat

SEQ ID NO: 95 - DNA, 20 bp, artificial
  human mitoNEET antisense
gtgcactaggataccgagct

SEQ ID NO: 96 - DNA, 20 bp, artificial
  human mitoNEET antisense
gaaggtgaatcagacagagg

SEQ ID NO: 97 - DNA, 20 bp, artificial
  human mitoNEET antisense
ctccaacaacggcagtacac

SEQ ID NO: 98 - DNA, 20 bp, artificial
  human mitoNEET antisense
cagacagaggtggtagtcat

SEQ ID NO: 99 - DNA, 20 bp, artificial
  human mitoNEET antisense
tcgatgatctttaacataaa

SEQ ID NO: 100 - DNA, 20 bp, artificial
  human mitoNEET antisense
tgtcaaaagcatgtactatc

SEQ ID NO: 101 - DNA, 20 bp, artificial
  human mitoNEET antisense
cccaaatcctccatgtcaaa

SEQ ID NO: 102 - DNA, 20 bp, artificial
  human mitoNEET antisense
aggtggtagtcattctaatt

SEQ ID NO: 103 - DNA, 20 bp, artificial
  human mitoNEET antisense
acgggttcgcgcggccggcg

SEQ ID NO: 104 - DNA, 20 bp, artificial
  human mitoNEET antisense
ttatttcgatgatctttaac

SEQ ID NO: 105 - DNA, 20 bp, artificial
  human mitoNEET antisense
agcatgtactatcttggggt

SEQ ID NO: 106 - DNA, 20 bp, artificial
  human mitoNEET antisense
ggtggtagtcattctaatta

SEQ ID NO: 107 - DNA, 20 bp, artificial
  human mitoNEET antisense
gtttgcaatataccacattt

SEQ ID NO: 108 - DNA, 20 bp, artificial
  human mitoNEET antisense
acaaatgtgcaccacgatgt

SEQ ID NO: 109 - DNA, 20 bp, artificial
  human mitoNEET antisense
tcaaacgggttcgcgcggcc

SEQ ID NO: 110 - DNA, 20 bp, artificial
  human mitoNEET antisense
tgtgcaccacgatgtttcaa

SEQ ID NO: 111 - DNA, 20 bp, artificial
  human mitoNEET antisense
cgggttcgcgcggccggcga

SEQ ID NO: 112 - DNA, 20 bp, artificial
  human mitoNEET antisense
agacagaggtggtagtcatt

SEQ ID NO: 113 - DNA, 20 bp, artificial
  human mitoNEET antisense
gatccattcaactcgtacgc

SEQ ID NO: 114 - DNA, 20 bp, artificial
  human mitoNEET antisense
catgtcaaaagcatgtacta

SEQ ID NO: 115 - DNA, 20 bp, artificial
  human mitoNEET antisense
cttcgttatgttttgtgtga

SEQ ID NO: 116 - DNA, 20 bp, artificial
  human mitoNEET antisense
ggttgtctttctggatgtga

SEQ ID NO: 117 - DNA, 20 bp, artificial
  human mitoNEET antisense
ctcaaacgggttcgcgcggc

SEQ ID NO: 118 - DNA, 20 bp, artificial
  human mitoNEET antisense
tgtgcactaggataccgagc

SEQ ID NO: 119 - DNA, 20 bp, artificial
  human mitoNEET antisense
tgcaatataccacatttaga

SEQ ID NO: 120 - DNA, 20 bp, artificial
  human mitoNEET antisense
caaatgtgcaccacgatgtt

SEQ ID NO: 121 - DNA, 20 bp, artificial
  human mitoNEET antisense
acctccaacaacggcagtac

SEQ ID NO: 122 - DNA, 20 bp, artificial
  human mitoNEET antisense
tcttttttcttgatgatcag

SEQ ID NO: 123 - DNA, 20 bp, artificial
  human mitoNEET antisense
gaatcagacagaggtggtag

SEQ ID NO: 124 - DNA, 20 bp, artificial
  human mitoNEET antisense
tttatttcgatgatctttaa

SEQ ID NO: 125 - DNA, 20 bp, artificial
  human mitoNEET antisense
aacgggttcgcgcggccggc

SEQ ID NO: 126 - DNA, 20 bp, artificial
  human mitoNEET antisense
gtcagactcatggcgccgtc

SEQ ID NO: 127 - DNA, 20 bp, artificial
  human mitoNEET antisense
cgatgatctttaacataaaa

SEQ ID NO: 128 - DNA, 20 bp, artificial
  human mitoNEET antisense
gttgtctttctggatgtgaa

SEQ ID NO: 129 - DNA, 20 bp, artificial
  human mitoNEET antisense
gcgagagtaaaggtgccagc

SEQ ID NO: 130 - DNA, 20 bp, artificial
  human mitoNEET antisense
ccattcaactcgtacgctgg

SEQ ID NO: 131 - DNA, 20 bp, artificial
  human mitoNEET antisense
tcaaaagcatgtactatctt

SEQ ID NO: 132 - DNA, 20 bp, artificial
  human mitoNEET antisense
aaacaaatgtgcaccacgat

SEQ ID NO: 133 - DNA, 20 bp, artificial
  human mitoNEET antisense
aaggtgaatcagacagaggt

SEQ ID NO: 134 - DNA, 20 bp, artificial
  human mitoNEET antisense
ataccacatttagaatccag

SEQ ID NO: 135 - DNA, 20 bp, artificial
  human mitoNEET antisense
gccggcgagagtaaaggtgc

SEQ ID NO: 136 - DNA, 20 bp, artificial
  human mitoNEET antisense
ttcgcgcggccggcgagagt

SEQ ID NO: 137 - DNA, 20 bp, artificial
  human mitoNEET antisense
gttcgcgcggccggcgagag

SEQ ID NO: 138 - DNA, 20 bp, artificial
  human mitoNEET antisense
tctttaacataaaatctttt

SEQ ID NO: 139 - DNA, 20 bp, artificial
  human mitoNEET antisense
gcatgtactatcttggggtt

SEQ ID NO: 140 - DNA, 20 bp, artificial
  human mitoNEET antisense
gcaatataccacatttagaa

SEQ ID NO: 141 - DNA, 20 bp, artificial
  human mitoNEET antisense
tttttttttttttttgttta

SEQ ID NO: 142 - DNA, 20 bp, artificial
  human mitoNEET antisense
cgcgcggccggcgagagtaa

SEQ ID NO: 143 - DNA, 20 bp, artificial
  human mitoNEET antisense
tcgcgcggccggcgagagta

SEQ ID NO: 144 - DNA, 20 bp, artificial
  human mitoNEET antisense
tcagactcatggcgccgtcg

SEQ ID NO: 145 - DNA, 20 bp, artificial
  human mitoNEET antisense
cgatccattcaactcgtacg

SEQ ID NO: 146 - DNA, 20 bp, artificial
  human mitoNEET antisense
atctttaacataaaatcttt

SEQ ID NO: 147 - DNA, 20 bp, artificial
  human mitoNEET antisense
taaacaaatgtgcaccacga

SEQ ID NO: 148 - DNA, 20 bp, artificial
  human mitoNEET antisense
gatctttaacataaaatctt

SEQ ID NO: 149 - DNA, 20 bp, artificial
  human mitoNEET antisense
ttcttttttcttgatgatca

SEQ ID NO: 150 - DNA, 20 bp, artificial
  human mitoNEET antisense
tttaagtttcttttttcttg

SEQ ID NO: 151 - DNA, 20 bp, artificial
  human mitoNEET antisense
tataccacatttagaatcca

SEQ ID NO: 152 - DNA, 20 bp, artificial
  human mitoNEET antisense
ttctaattaaacaatcaggt

SEQ ID NO: 153 - DNA, 20 bp, artificial
  human mitoNEET antisense
tgcactaggataccgagctc

SEQ ID NO: 154 - DNA, 20 bp, artificial
  human mitoNEET antisense
ctttaacataaaatcttttg

SEQ ID NO: 155 - DNA, 20 bp, artificial
  human mitoNEET antisense
gatgatctttaacataaaat

SEQ ID NO: 156 - DNA, 20 bp, artificial
  human mitoNEET antisense
tctcccaaatcctccatgtc

SEQ ID NO: 157 - DNA, 20 bp, artificial
  human mitoNEET antisense
agtttgcaatataccacatt

SEQ ID NO: 158 - DNA, 20 bp, artificial
  human mitoNEET antisense
ccagcgaaggtgaatcagac

SEQ ID NO: 159 - DNA, 20 bp, artificial
  human mitoNEET antisense
catttagaatccagcgaagg

SEQ ID NO: 160 - DNA, 20 bp, artificial
  human mitoNEET antisense
gcgcggccggcgagagtaaa

SEQ ID NO: 161 - DNA, 20 bp, artificial
  human mitoNEET antisense
tttaacataaaatcttttgt

SEQ ID NO: 162 - DNA, 20 bp, artificial
  human mitoNEET antisense
ctttatttcgatgatcttta

SEQ ID NO: 163 - DNA, 20 bp, artificial
  human mitoNEET antisense
aagcatgtactatcttgggg

SEQ ID NO: 164 - DNA, 20 bp, artificial
  human mitoNEET antisense
ggcccacattgtctccagtc

SEQ ID NO: 165 - DNA, 20 bp, artificial
  human mitoNEET antisense
cttttttcttgatgatcaga

SEQ ID NO: 166 - DNA, 20 bp, artificial
  human mitoNEET antisense
tgtttaaacaaatgtgcacc

SEQ ID NO: 167 - DNA, 20 bp, artificial
  human mitoNEET antisense
cgagagtaaaggtgccagcg

SEQ ID NO: 168 - DNA, 20 bp, artificial
  human mitoNEET antisense
ggcgagagtaaaggtgccag

SEQ ID NO: 169 - DNA, 20 bp, artificial
  human mitoNEET antisense
gtacgctggaactggaagtc

SEQ ID NO: 170 - DNA, 20 bp, artificial
  human mitoNEET antisense
gtggtagtcattctaattaa

SEQ ID NO: 171 - DNA, 20 bp, artificial
  human mitoNEET antisense
ctaaagcaccgactccgcga

SEQ ID NO: 172 - DNA, 20 bp, artificial
  human mitoNEET antisense
tgatctttaacataaaatct

SEQ ID NO: 173 - DNA, 20 bp, artificial
  human mitoNEET antisense
caaatcctccatgtcaaaag

SEQ ID NO: 174 - DNA, 20 bp, artificial
  human mitoNEET antisense
ggccggcgagagtaaaggtg

SEQ ID NO: 175 - DNA, 20 bp, artificial
  human mitoNEET antisense
gtcccagcagcaatggtaac

SEQ ID NO: 176 - DNA, 20 bp, artificial
  human mitoNEET antisense
atataccacatttagaatcc

SEQ ID NO: 177 - DNA, 20 bp, artificial
  human mitoNEET antisense
gtgcaccacgatgtttcaac

SEQ ID NO: 178 - DNA, 20 bp, artificial
  human mitoNEET antisense
tactatcttggggttgtctt

SEQ ID NO: 179 - DNA, 20 bp, artificial
  human mitoNEET antisense
tacgctggaactggaagtca

SEQ ID NO: 180 - DNA, 20 bp, artificial
  human mitoNEET antisense
atgatctttaacataaaatc

SEQ ID NO: 181 - DNA, 20 bp, artificial
  human mitoNEET antisense
aatcagacagaggtggtagt

SEQ ID NO: 182 - DNA, 20 bp, artificial
  human mitoNEET antisense
tcccagcagcaatggtaact

SEQ ID NO: 183 - DNA, 20 bp, artificial
  human mitoNEET antisense
ttaagtttcttttttcttga

SEQ ID NO: 184 - DNA, 20 bp, artificial
  human mitoNEET antisense
tttgcaatataccacattta

SEQ ID NO: 185 - DNA, 20 bp, artificial
  human mitoNEET antisense
agtaaaggtgccagcggcgt

SEQ ID NO: 186 - DNA, 20 bp, artificial
  human mitoNEET antisense
acgctggaactggaagtcag

SEQ ID NO: 187 - DNA, 20 bp, artificial
  human mitoNEET antisense
gcgatccattcaactcgtac

SEQ ID NO: 188 - DNA, 20 bp, artificial
  human mitoNEET antisense
tggacctccaacaacggcag

SEQ ID NO: 189 - DNA, 20 bp, artificial
  human mitoNEET antisense
actaaagcaccgactccgcg

SEQ ID NO: 190 - DNA, 20 bp, artificial
  human mitoNEET antisense
aactcgtacgctggaactgg

SEQ ID NO: 191 - DNA, 20 bp, artificial
  human mitoNEET antisense
tcttggggttgtctttctgg

SEQ ID NO: 192 - DNA, 20 bp, artificial
  human mitoNEET antisense
attctaattaaacaatcagg

SEQ ID NO: 193 - DNA, 20 bp, artificial
  human mitoNEET antisense
gtaaaggtgccagcggcgta

SEQ ID NO: 194 - DNA, 20 bp, artificial
  human mitoNEET antisense
gcgtgtgcactaggataccg

SEQ ID NO: 195 - DNA, 20 bp, artificial
  human mitoNEET antisense
cagtttgcaatataccacat

SEQ ID NO: 196 - DNA, 20 bp, artificial
  human mitoNEET antisense
atttaagtttcttttttctt

SEQ ID NO: 197 - DNA, 20 bp, artificial
  human mitoNEET antisense
cagaggtggtagtcattcta

SEQ ID NO: 198 - DNA, 20 bp, artificial
  human mitoNEET antisense
aacaaatgtgcaccacgatg

SEQ ID NO: 199 - DNA, 20 bp, artificial
  human mitoNEET antisense
ttttttttttttttgtttaa

SEQ ID NO: 200 - DNA, 20 bp, artificial
  human mitoNEET antisense
cgcggccggcgagagtaaag

SEQ ID NO: 201 - DNA, 20 bp, artificial
  human mitoNEET antisense
gactcatggcgccgtcgctt

SEQ ID NO: 202 - DNA, 20 bp, artificial
  human mitoNEET antisense
cgctggaactggaagtcaga

SEQ ID NO: 203 - DNA, 20 bp, artificial
  human mitoNEET antisense
gggaactttttggacctcca

SEQ ID NO: 204 - DNA, 20 bp, artificial
  human mitoNEET antisense
atcagacagaggtggtagtc

SEQ ID NO: 205 - DNA, 20 bp, artificial
  human mitoNEET antisense
caatataccacatttagaat

SEQ ID NO: 206 - DNA, 20 bp, artificial
  human mitoNEET antisense
gagtaaaggtgccagcggcg

SEQ ID NO: 207 - DNA, 20 bp, artificial
  human mitoNEET antisense
gctttatttcgatgatcttt

SEQ ID NO: 208 - DNA, 20 bp, artificial
  human mitoNEET antisense
ttggacctccaacaacggca

SEQ ID NO: 209 - DNA, 20 bp, artificial
  human mitoNEET antisense
gtagtcattctaattaaaca

SEQ ID NO: 210 - DNA, 20 bp, artificial
  human mitoNEET antisense
caccacgatgtttcaacaag

SEQ ID NO: 211 - DNA, 20 bp, artificial
  human mitoNEET antisense
ttgtctttctggatgtgaag

SEQ ID NO: 212 - DNA, 20 bp, artificial
  human mitoNEET antisense
cttggggttgtctttctgga

SEQ ID NO: 213 - DNA, 20 bp, artificial
  human mitoNEET antisense
actatcttggggttgtcttt

SEQ ID NO: 214 - DNA, 20 bp, artificial
  human mitoNEET antisense
gtactatcttggggttgtct

SEQ ID NO: 215 - DNA, 20 bp, artificial
  human mitoNEET antisense
cggccggcgagagtaaaggt

SEQ ID NO: 216 - DNA, 20 bp, artificial
  human mitoNEET antisense
ccaaatcctccatgtcaaaa

SEQ ID NO: 217 - DNA, 20 bp, artificial
  human mitoNEET antisense
tccaacaacggcagtacaca

SEQ ID NO: 218 - DNA, 20 bp, artificial
  human mitoNEET antisense
tgggaactttttggacctcc

SEQ ID NO: 219 - DNA, 20 bp, artificial
  human mitoNEET antisense
tctaattaaacaatcaggta

SEQ ID NO: 220 - DNA, 20 bp, artificial
  human mitoNEET antisense
ggttcgcgcggccggcgaga

SEQ ID NO: 221 - DNA, 20 bp, artificial
  human mitoNEET antisense
ttaacataaaatcttttgta

SEQ ID NO: 222 - DNA, 20 bp, artificial
  human mitoNEET antisense
atctcccaaatcctccatgt

SEQ ID NO: 223 - DNA, 20 bp, artificial
  human mitoNEET antisense
gagggcccacattgtctcca

SEQ ID NO: 224 - DNA, 20 bp, artificial
  human mitoNEET antisense
tactaaagcaccgactccgc

SEQ ID NO: 225 - DNA, 20 bp, artificial
  human mitoNEET antisense
gagagtaaaggtgccagcgg

SEQ ID NO: 226 - DNA, 20 bp, artificial
  human mitoNEET antisense
cgtgtgcactaggataccga

SEQ ID NO: 227 - DNA, 20 bp, artificial
  human mitoNEET antisense
aacaacggcagtacacagct

SEQ ID NO: 228 - DNA, 20 bp, artificial
  human mitoNEET antisense
gacctccaacaacggcagta

SEQ ID NO: 229 - DNA, 20 bp, artificial
  human mitoNEET antisense
agactcatggcgccgtcgct

SEQ ID NO: 230 - DNA, 20 bp, artificial
  human mitoNEET antisense
acaacggcagtacacagctt

SEQ ID NO: 231 - DNA, 20 bp, artificial
  human mitoNEET antisense
tttcttttttcttgatgatc

SEQ ID NO: 232 - DNA, 20 bp, artificial
  human mitoNEET antisense
aaagcatgtactatcttggg

SEQ ID NO: 233 - DNA, 20 bp, artificial
  human mitoNEET antisense
caaaagcatgtactatcttg

SEQ ID NO: 234 - DNA, 20 bp, artificial
  human mitoNEET antisense
gaggtggtagtcattctaat

SEQ ID NO: 235 - DNA, 20 bp, artificial
  human mitoNEET antisense
aagctgcagtttgcaatata

SEQ ID NO: 236 - DNA, 20 bp, artificial
  human mitoNEET antisense
ctttatctcccaaatcctcc

SEQ ID NO: 237 - DNA, 20 bp, artificial
  human mitoNEET antisense
atcttggggttgtctttctg

SEQ ID NO: 238 - DNA, 20 bp, artificial
  human mitoNEET antisense
taagtttcttttttcttgat

SEQ ID NO: 239 - DNA, 20 bp, artificial
  human mitoNEET antisense
ttaaacaaatgtgcaccacg

SEQ ID NO: 240 - DNA, 20 bp, artificial
  human mitoNEET antisense
caacggcagtacacagcttt

SEQ ID NO: 241 - DNA, 20 bp, artificial
  human mitoNEET antisense
agggcccacattgtctccag

SEQ ID NO: 242 - DNA, 20 bp, artificial
  human mitoNEET antisense
agaggtggtagtcattctaa

SEQ ID NO: 243 - DNA, 20 bp, artificial
  human mitoNEET antisense
tatcatagctttatttcgat

SEQ ID NO: 244 - DNA, 20 bp, artificial
  human mitoNEET antisense
caacaacggcagtacacagc

SEQ ID NO: 245 - DNA, 20 bp, artificial
  human mitoNEET antisense
agagggcccacattgtctcc

SEQ ID NO: 246 - DNA, 20 bp, artificial
  human mitoNEET antisense
aaacaatcaggtaacttcac

SEQ ID NO: 247 - DNA, 20 bp, artificial
  human mitoNEET antisense
gcaaaggcgtgtgcactagg

SEQ ID NO: 248 - DNA, 20 bp, artificial
  human mitoNEET antisense
agcaatggtaactgctgcga

SEQ ID NO: 249 - DNA, 20 bp, artificial
  human mitoNEET antisense
tatcttggggttgtctttct

SEQ ID NO: 250 - DNA, 20 bp, artificial
  human mitoNEET antisense
ggcagtacacagctttatct

SEQ ID NO: 251 - DNA, 20 bp, artificial
  human mitoNEET antisense
acagaggtggtagtcattct

SEQ ID NO: 252 - DNA, 20 bp, artificial
  human mitoNEET antisense
acatttagaatccagcgaag

SEQ ID NO: 253 - DNA, 20 bp, artificial
  human mitoNEET antisense
tgtccatttaagtttctttt

SEQ ID NO: 254 - DNA, 20 bp, artificial
  human mitoNEET antisense
agctgcagtttgcaatatac

SEQ ID NO: 255 - DNA, 20 bp, artificial
  human mitoNEET antisense
accacgatgtttcaacaaga

SEQ ID NO: 256 - DNA, 20 bp, artificial
  human mitoNEET antisense
gcaatggtaactgctgcgat

SEQ ID NO: 257 - DNA, 20 bp, artificial
  human mitoNEET antisense
aatataccacatttagaatc

SEQ ID NO: 258 - DNA, 20 bp, artificial
  human mitoNEET antisense
tgtcccagcagcaatggtaa

SEQ ID NO: 259 - DNA, 20 bp, artificial
  human mitoNEET antisense
ctgtcccagcagcaatggta

SEQ ID NO: 260 - DNA, 20 bp, artificial
  human mitoNEET antisense
ggtttatcatagctttattt

SEQ ID NO: 261 - DNA, 20 bp, artificial
  human mitoNEET antisense
tttatctcccaaatcctcca

SEQ ID NO: 262 - DNA, 20 bp, artificial
  human mitoNEET antisense
ttgtttaaacaaatgtgcac

SEQ ID NO: 263 - DNA, 20 bp, artificial
  human mitoNEET antisense
gtgtgcactaggataccgag

SEQ ID NO: 264 - DNA, 20 bp, artificial
  human mitoNEET antisense
aggtttatcatagctttatt

SEQ ID NO: 265 - DNA, 20 bp, artificial
  human mitoNEET antisense
ttcgttatgttttgtgtgag

SEQ ID NO: 266 - DNA, 20 bp, artificial
  human mitoNEET antisense
attaaacaatcaggtaactt

SEQ ID NO: 267 - DNA, 20 bp, artificial
  human mitoNEET antisense
cattctaattaaacaatcag

SEQ ID NO: 268 - DNA, 20 bp, artificial
  human mitoNEET antisense
tttaaacaaatgtgcaccac

SEQ ID NO: 269 - DNA, 20 bp, artificial
  human mitoNEET antisense
aggtgccagcggcgtactaa

SEQ ID NO: 270 - DNA, 20 bp, artificial
  human mitoNEET antisense
tgcagcatcaaaagtgtcca

SEQ ID NO: 271 - DNA, 20 bp, artificial
  human mitoNEET antisense
agtcattctaattaaacaat

SEQ ID NO: 272 - DNA, 20 bp, artificial
  human mitoNEET antisense
ttgcaatataccacatttag

SEQ ID NO: 273 - DNA, 20 bp, artificial
  human mitoNEET antisense
tttttttttttttgtttaaa

SEQ ID NO: 274 - DNA, 20 bp, artificial
  human mitoNEET antisense
cgccgtcgcttgcaaaggcg

SEQ ID NO: 275 - DNA, 20 bp, artificial
  human mitoNEET antisense
tgcgatccattcaactcgta

SEQ ID NO: 276 - DNA, 20 bp, artificial
  human mitoNEET antisense
catgtactatcttggggttg

SEQ ID NO: 277 - DNA, 20 bp, artificial
  human mitoNEET antisense
ttatgttttgtgtgagcccc

SEQ ID NO: 278 - DNA, 20 bp, artificial
  human mitoNEET antisense
gctggaactggaagtcagac

SEQ ID NO: 279 - DNA, 20 bp, artificial
  human mitoNEET antisense
ggacctccaacaacggcagt

SEQ ID NO: 280 - DNA, 20 bp, artificial
  human mitoNEET antisense
atcacagaatgggaactttt

SEQ ID NO: 281 - DNA, 20 bp, artificial
  human mitoNEET antisense
caaacgggttcgcgcggccg

SEQ ID NO: 282 - DNA, 20 bp, artificial
  human mitoNEET antisense
ttttgtaagctagataacca

SEQ ID NO: 283 - DNA, 20 bp, artificial
  human mitoNEET antisense
taacataaaatcttttgtaa

SEQ ID NO: 284 - DNA, 20 bp, artificial
  human mitoNEET antisense
ttatcatagctttatttcga

SEQ ID NO: 285 - DNA, 20 bp, artificial
  human mitoNEET antisense
ctatcttggggttgtctttc

SEQ ID NO: 286 - DNA, 20 bp, artificial
  human mitoNEET antisense
tagtcattctaattaaacaa

SEQ ID NO: 287 - DNA, 20 bp, artificial
  human mitoNEET antisense
gacagaggtggtagtcattc

SEQ ID NO: 288 - DNA, 20 bp, artificial
  human mitoNEET antisense
aacaatcaggtaacttcacg

SEQ ID NO: 289 - DNA, 20 bp, artificial
  human mitoNEET antisense
tcattctaattaaacaatca

SEQ ID NO: 290 - DNA, 20 bp, artificial
  human mitoNEET antisense
ggtagtcattctaattaaac

SEQ ID NO: 291 - DNA, 20 bp, artificial
  human mitoNEET antisense
gtgaaagctgcagtttgcaa

SEQ ID NO: 292 - DNA, 20 bp, artificial
  human mitoNEET antisense
tttttttttttgtttaaaca

SEQ ID NO: 293 - DNA, 20 bp, artificial
  human mitoNEET antisense
gggttcgcgcggccggcgag

SEQ ID NO: 294 - DNA, 20 bp, artificial
  human mitoNEET antisense
agctttatttcgatgatctt

SEQ ID NO: 295 - DNA, 20 bp, artificial
  human mitoNEET antisense
gtccatttaagtttcttttt

SEQ ID NO: 296 - DNA, 20 bp, artificial
  human mitoNEET antisense
aaagctgcagtttgcaatat

SEQ ID NO: 297 - DNA, 20 bp, artificial
  human mitoNEET antisense
tgtgaaagctgcagtttgca

SEQ ID NO: 298 - DNA, 20 bp, artificial
  human mitoNEET antisense
ctgcgatccattcaactcgt

SEQ ID NO: 299 - DNA, 20 bp, artificial
  human mitoNEET antisense
ggaactttttggacctccaa

SEQ ID NO: 300 - DNA, 20 bp, artificial
  human mitoNEET antisense
ctaattaaacaatcaggtaa

SEQ ID NO: 301 - DNA, 20 bp, artificial
  human mitoNEET antisense
ggtgccagcggcgtactaaa

SEQ ID NO: 302 - DNA, 20 bp, artificial
  human mitoNEET antisense
ccgtcgcttgcaaaggcgtg

SEQ ID NO: 303 - DNA, 20 bp, artificial
  human mitoNEET antisense
tatgttttgtgtgagcccca

SEQ ID NO: 304 - DNA, 20 bp, artificial
  human mitoNEET antisense
cagagggcccacattgtctc

SEQ ID NO: 305 - DNA, 20 bp, artificial
  human mitoNEET antisense
aattaaacaatcaggtaact

SEQ ID NO: 306 - DNA, 20 bp, artificial
  human mitoNEET antisense
gcggccggcgagagtaaagg

SEQ ID NO: 307 - DNA, 20 bp, artificial
  human mitoNEET antisense
ggcgtgtgcactaggatacc

SEQ ID NO: 308 - DNA, 20 bp, artificial
  human mitoNEET antisense
aagtcagactcatggcgccg

SEQ ID NO: 309 - DNA, 20 bp, artificial
  human mitoNEET antisense
ttatctcccaaatcctccat

SEQ ID NO: 310 - DNA, 20 bp, artificial
  human mitoNEET antisense
gtttcttttttcttgatgat

SEQ ID NO: 311 - DNA, 20 bp, artificial
  human mitoNEET antisense
catttaagtttcttttttct

SEQ ID NO: 312 - DNA, 20 bp, artificial
  human mitoNEET antisense
ggtaactgctgcgatccatt

SEQ ID NO: 313 - DNA, 20 bp, artificial
  human mitoNEET antisense
ccaacaacggcagtacacag

SEQ ID NO: 314 - DNA, 20 bp, artificial
  human mitoNEET antisense
catcacagaatgggaacttt

SEQ ID NO: 315 - DNA, 20 bp, artificial
  human mitoNEET antisense
cacatttagaatccagcgaa

SEQ ID NO: 316 - DNA, 20 bp, artificial
  human mitoNEET antisense
tttgtttaaacaaatgtgca

SEQ ID NO: 317 - DNA, 20 bp, artificial
  human mitoNEET antisense
tgcaaaggcgtgtgcactag

SEQ ID NO: 318 - DNA, 20 bp, artificial
  human mitoNEET antisense
gaagtcagactcatggcgcc

SEQ ID NO: 319 - DNA, 20 bp, artificial
  human mitoNEET antisense
tggtaactgctgcgatccat

SEQ ID NO: 320 - DNA, 20 bp, artificial
  human mitoNEET antisense
atggtaactgctgcgatcca

SEQ ID NO: 321 - DNA, 20 bp, artificial
  human mitoNEET antisense
tttgtaagctagataaccaa

SEQ ID NO: 322 - DNA, 20 bp, artificial
  human mitoNEET antisense
ctttctggatgtgaaggttt

SEQ ID NO: 323 - DNA, 20 bp, artificial
  human mitoNEET antisense
gctttatctcccaaatcctc

SEQ ID NO: 324 - DNA, 20 bp, artificial
  human mitoNEET antisense
gcagtacacagctttatctc

SEQ ID NO: 325 - DNA, 20 bp, artificial
  human mitoNEET antisense
gcgccgtcgcttgcaaaggc

SEQ ID NO: 326 - DNA, 20 bp, artificial
  human mitoNEET antisense
ttgtaagctagataaccaat

SEQ ID NO: 327 - DNA, 20 bp, artificial
  human mitoNEET antisense
aaggtttatcatagctttat

SEQ ID NO: 328 - DNA, 20 bp, artificial
  human mitoNEET antisense
tctggatgtgaaggtttatc

SEQ ID NO: 329 - DNA, 20 bp, artificial
  human mitoNEET antisense
tgtctttctggatgtgaagg

SEQ ID NO: 330 - DNA, 20 bp, artificial
  human mitoNEET antisense
tatctcccaaatcctccatg

SEQ ID NO: 331 - DNA, 20 bp, artificial
  human mitoNEET antisense
ccagcagcaatggtaactgc

SEQ ID NO: 332 - DNA, 20 bp, artificial
  human mitoNEET antisense
tttctggatgtgaaggttta

SEQ ID NO: 333 - DNA, 20 bp, artificial
  human mitoNEET antisense
gggcccacattgtctccagt

SEQ ID NO: 334 - DNA, 20 bp, artificial
  human mitoNEET antisense
agcatcaaaagtgtccattt

SEQ ID NO: 335 - DNA, 20 bp, artificial
  human mitoNEET antisense
tgatttgcagcatcaaaagt

SEQ ID NO: 336 - DNA, 20 bp, artificial
  human mitoNEET antisense
atgtgaaagctgcagtttgc

SEQ ID NO: 337 - DNA, 20 bp, artificial
  human mitoNEET antisense
ttctggatgtgaaggtttat

SEQ ID NO: 338 - DNA, 20 bp, artificial
  human mitoNEET antisense
cggcagtacacagctttatc

SEQ ID NO: 339 - DNA, 20 bp, artificial
  human mitoNEET antisense
tcgttatgttttgtgtgagc

SEQ ID NO: 340 - DNA, 20 bp, artificial
  human mitoNEET antisense
acaatcaggtaacttcacga

SEQ ID NO: 341 - DNA, 20 bp, artificial
  human mitoNEET antisense
tgaaagctgcagtttgcaat

SEQ ID NO: 342 - DNA, 20 bp, artificial
  human mitoNEET antisense
ccacgatgtttcaacaagac

SEQ ID NO: 343 - DNA, 20 bp, artificial
  human mitoNEET antisense
aaggtgccagcggcgtacta

SEQ ID NO: 344 - DNA, 20 bp, artificial
  human mitoNEET antisense
aggcgtgtgcactaggatac

SEQ ID NO: 345 - DNA, 20 bp, artificial
  human mitoNEET antisense
atttgcagcatcaaaagtgt

SEQ ID NO: 346 - DNA, 20 bp, artificial
  human mitoNEET antisense
gtttaaacaaatgtgcacca

SEQ ID NO: 347 - DNA, 20 bp, artificial
  human mitoNEET antisense
aaaagcatgtactatcttgg

SEQ ID NO: 348 - DNA, 20 bp, artificial
  human mitoNEET antisense
gaactttttggacctccaac

SEQ ID NO: 349 - DNA, 20 bp, artificial
  human mitoNEET antisense
gatgtttcaacaagacaaat

SEQ ID NO: 350 - DNA, 20 bp, artificial
  human mitoNEET antisense
acgatgtttcaacaagacaa

SEQ ID NO: 351 - DNA, 20 bp, artificial
  human mitoNEET antisense
ttttgtttaaacaaatgtgc

SEQ ID NO: 352 - DNA, 20 bp, artificial
  human mitoNEET antisense
agagtaaaggtgccagcggc

SEQ ID NO: 353 - DNA, 20 bp, artificial
  human mitoNEET antisense
agtcagactcatggcgccgt

SEQ ID NO: 354 - DNA, 20 bp, artificial
  human mitoNEET antisense
ggaactggaagtcagactca

SEQ ID NO: 355 - DNA, 20 bp, artificial
  human mitoNEET antisense
cagctttatctcccaaatcc

SEQ ID NO: 356 - DNA, 20 bp, artificial
  human mitoNEET antisense
gtaacttcacgacaagctga

SEQ ID NO: 357 - DNA, 20 bp, artificial
  human mitoNEET antisense
taaaggtgccagcggcgtac

SEQ ID NO: 358 - DNA, 20 bp, artificial
  human mitoNEET antisense
gcttgcaaaggcgtgtgcac

SEQ ID NO: 359 - DNA, 20 bp, artificial
  human mitoNEET antisense
tcagagggcccacattgtct

SEQ ID NO: 360 - DNA, 20 bp, artificial
  human mitoNEET antisense
ttttttcttgatgatcagag

SEQ ID NO: 361 - DNA, 20 bp, artificial
  human mitoNEET antisense
ttaaacaatcaggtaacttc

SEQ ID NO: 362 - DNA, 20 bp, artificial
  human mitoNEET antisense
gaaagctgcagtttgcaata

SEQ ID NO: 363 - DNA, 20 bp, artificial
  human mitoNEET antisense
cgatgtttcaacaagacaaa

SEQ ID NO: 364 - DNA, 20 bp, artificial
  human mitoNEET antisense
aaagcaccgactccgcgatc

SEQ ID NO: 365 - DNA, 20 bp, artificial
  human mitoNEET antisense
aaaggtgccagcggcgtact

SEQ ID NO: 366 - DNA, 20 bp, artificial
  human mitoNEET antisense
atgttttgtgtgagccccat

SEQ ID NO: 367 - DNA, 20 bp, artificial
  human mitoNEET antisense
aagtttcttttttcttgatg

SEQ ID NO: 368 - DNA, 20 bp, artificial
  human mitoNEET antisense
tggtagtcattctaattaaa

SEQ ID NO: 369 - DNA, 20 bp, artificial
  human mitoNEET antisense
ttttttttttgtttaaacaa

SEQ ID NO: 370 - DNA, 20 bp, artificial
  human mitoNEET antisense
ctggaactggaagtcagact

SEQ ID NO: 371 - DNA, 20 bp, artificial
  human mitoNEET antisense
caatggtaactgctgcgatc

SEQ ID NO: 372 - DNA, 20 bp, artificial
  human mitoNEET antisense
ataaaatcttttgtaagcta

SEQ ID NO: 373 - DNA, 20 bp, artificial
  human mitoNEET antisense
gaactggaagtcagactcat

SEQ ID NO: 374 - DNA, 20 bp, artificial
  human mitoNEET antisense
atcatagctttatttcgatg

SEQ ID NO: 375 - DNA, 20 bp, artificial
  human mitoNEET antisense
tttatcatagctttatttcg

SEQ ID NO: 376 - DNA, 20 bp, artificial
  human mitoNEET antisense
cagactcatggcgccgtcgc

SEQ ID NO: 377 - DNA, 20 bp, artificial
  human mitoNEET antisense
cttttgtaagctagataacc

SEQ ID NO: 378 - DNA, 20 bp, artificial
  human mitoNEET antisense
atagctttatttcgatgatc

SEQ ID NO: 379 - DNA, 20 bp, artificial
  human mitoNEET antisense
ccatcacagaatgggaactt

SEQ ID NO: 380 - DNA, 20 bp, artificial
  human mitoNEET antisense
tagctttatttcgatgatct

SEQ ID NO: 381 - DNA, 20 bp, artificial
  human mitoNEET antisense
gaaggtttatcatagcttta

SEQ ID NO: 382 - DNA, 20 bp, artificial
  human mitoNEET antisense
cagtacacagctttatctcc

SEQ ID NO: 383 - DNA, 20 bp, artificial
  human mitoNEET antisense
aacggcagtacacagcttta

SEQ ID NO: 384 - DNA, 20 bp, artificial
  human mitoNEET antisense
gtgtccatttaagtttcttt

SEQ ID NO: 385 - DNA, 20 bp, artificial
  human mitoNEET antisense
aaggcgtgtgcactaggata

SEQ ID NO: 386 - DNA, 20 bp, artificial
  human mitoNEET antisense
cccagcagcaatggtaactg

SEQ ID NO: 387 - DNA, 20 bp, artificial
  human mitoNEET antisense
tgtaagctagataaccaatt

SEQ ID NO: 388 - DNA, 20 bp, artificial
  human mitoNEET antisense
aacataaaatcttttgtaag

SEQ ID NO: 389 - DNA, 20 bp, artificial
  human mitoNEET antisense
gtcattctaattaaacaatc

SEQ ID NO: 390 - DNA, 20 bp, artificial
  human mitoNEET antisense
gcatcaaaagtgtccattta

SEQ ID NO: 391 - DNA, 20 bp, artificial
  human mitoNEET antisense
ttttgtgtgagccccatcac

SEQ ID NO: 392 - DNA, 20 bp, artificial
  human mitoNEET antisense
caatcaggtaacttcacgac

SEQ ID NO: 393 - DNA, 20 bp, artificial
  human mitoNEET antisense
taaacaatcaggtaacttca

SEQ ID NO: 394 - DNA, 20 bp, artificial
  human mitoNEET antisense
tggaagtcagactcatggcg

SEQ ID NO: 395 - DNA, 20 bp, artificial
  human mitoNEET antisense
acataaaatcttttgtaagc

SEQ ID NO: 396 - DNA, 20 bp, artificial
  human mitoNEET antisense
gcagtttgcaatataccaca

SEQ ID NO: 397 - DNA, 20 bp, artificial
  human mitoNEET antisense
tggaactggaagtcagactc

SEQ ID NO: 398 - DNA, 20 bp, artificial
  human mitoNEET antisense
tcacgacaagctgatttgca

SEQ ID NO: 399 - DNA, 20 bp, artificial
  human mitoNEET antisense
taacttcacgacaagctgat

SEQ ID NO: 400 - DNA, 20 bp, artificial
  human mitoNEET antisense
ttttttttttttgtttaaac

SEQ ID NO: 401 - DNA, 20 bp, artificial
  human mitoNEET antisense
acacagctttatctcccaaa

SEQ ID NO: 402 - DNA, 20 bp, artificial
  human mitoNEET antisense
tctttctggatgtgaaggtt

SEQ ID NO: 403 - DNA, 20 bp, artificial
  human mitoNEET antisense
agtacacagctttatctccc

SEQ ID NO: 404 - DNA, 20 bp, artificial
  human mitoNEET antisense
cgttatgttttgtgtgagcc

SEQ ID NO: 405 - DNA, 20 bp, artificial
  human mitoNEET antisense
cagcagcaatggtaactgct

SEQ ID NO: 406 - DNA, 20 bp, artificial
  human mitoNEET antisense
cacagctttatctcccaaat

SEQ ID NO: 407 - DNA, 20 bp, artificial
  human mitoNEET antisense
ggtaacttcacgacaagctg

SEQ ID NO: 408 - DNA, 20 bp, artificial
  human mitoNEET antisense
cacgatgtttcaacaagaca

SEQ ID NO: 409 - DNA, 20 bp, artificial
  human mitoNEET antisense
ttttttgtttaaacaaatgt

SEQ ID NO: 410 - DNA, 20 bp, artificial
  human mitoNEET antisense
aaacgggttcgcgcggccgg

SEQ ID NO: 411 - DNA, 20 bp, artificial
  human mitoNEET antisense
agctttatctcccaaatcct

SEQ ID NO: 412 - DNA, 20 bp, artificial
  human mitoNEET antisense
gatgatcagagggcccacat

SEQ ID NO: 413 - DNA, 20 bp, artificial
  human mitoNEET antisense
tgatgatcagagggcccaca

SEQ ID NO: 414 - DNA, 20 bp, artificial
  human mitoNEET antisense
tttttgtttaaacaaatgtg

SEQ ID NO: 415 - DNA, 20 bp, artificial
  human mitoNEET antisense
aaaggcgtgtgcactaggat

SEQ ID NO: 416 - DNA, 20 bp, artificial
  human mitoNEET antisense
ctggaagtcagactcatggc

SEQ ID NO: 417 - DNA, 20 bp, artificial
  human mitoNEET antisense
acggcagtacacagctttat

SEQ ID NO: 418 - DNA, 20 bp, artificial
  human mitoNEET antisense
ctgcagtttgcaatatacca

SEQ ID NO: 419 - DNA, 20 bp, artificial
  human mitoNEET antisense
gctgcgatccattcaactcg

SEQ ID NO: 420 - DNA, 20 bp, artificial
  human mitoNEET antisense
aaccaattgcagctgtccca

SEQ ID NO: 421 - DNA, 20 bp, artificial
  human mitoNEET antisense
acagctttatctcccaaatc

SEQ ID NO: 422 - DNA, 20 bp, artificial
  human mitoNEET antisense
tttttggacctccaacaacg

SEQ ID NO: 423 - DNA, 20 bp, artificial
  human mitoNEET antisense
aactttttggacctccaaca

SEQ ID NO: 424 - DNA, 20 bp, artificial
  human mitoNEET antisense
gcagcaatggtaactgctgc

SEQ ID NO: 425 - DNA, 20 bp, artificial
  human mitoNEET antisense
gctgtcccagcagcaatggt

SEQ ID NO: 426 - DNA, 20 bp, artificial
  human mitoNEET antisense
gtacacagctttatctccca

SEQ ID NO: 427 - DNA, 20 bp, artificial
  human mitoNEET antisense
agaatgggaactttttggac

SEQ ID NO: 428 - DNA, 20 bp, artificial
  human mitoNEET antisense
atcagagggcccacattgtc

SEQ ID NO: 429 - DNA, 20 bp, artificial
  human mitoNEET antisense
gctgcagtttgcaatatacc

SEQ ID NO: 430 - DNA, 20 bp, artificial
  human mitoNEET antisense
taaagcaccgactccgcgat

SEQ ID NO: 431 - DNA, 20 bp, artificial
  human mitoNEET antisense
actcatggcgccgtcgcttg

SEQ ID NO: 432 - DNA, 20 bp, artificial
  human mitoNEET antisense
gtaactgctgcgatccattc

SEQ ID NO: 433 - DNA, 20 bp, artificial
  human mitoNEET antisense
agataaccaattgcagctgt

SEQ ID NO: 434 - DNA, 20 bp, artificial
  human mitoNEET antisense
ttcacgacaagctgatttgc

SEQ ID NO: 435 - DNA, 20 bp, artificial
  human mitoNEET antisense
gtactaaagcaccgactccg

SEQ ID NO: 436 - DNA, 20 bp, artificial
  human mitoNEET antisense
caaaggcgtgtgcactagga

SEQ ID NO: 437 - DNA, 20 bp, artificial
  human mitoNEET antisense
cttgcaaaggcgtgtgcact

SEQ ID NO: 438 - DNA, 20 bp, artificial
  human mitoNEET antisense
taaccaattgcagctgtccc

SEQ ID NO: 439 - DNA, 20 bp, artificial
  human mitoNEET antisense
agtgtccatttaagtttctt

SEQ ID NO: 440 - DNA, 20 bp, artificial
  human mitoNEET antisense
agcagcaatggtaactgctg

SEQ ID NO: 441 - DNA, 20 bp, artificial
  human mitoNEET antisense
aaagtgtccatttaagtttc

SEQ ID NO: 442 - DNA, 20 bp, artificial
  human mitoNEET antisense
gatttgcagcatcaaaagtg

SEQ ID NO: 443 - DNA, 20 bp, artificial
  human mitoNEET antisense
aatcaggtaacttcacgaca

SEQ ID NO: 444 - DNA, 20 bp, artificial
  human mitoNEET antisense
taattaaacaatcaggtaac

SEQ ID NO: 445 - DNA, 20 bp, artificial
  human mitoNEET antisense
gggttgtctttctggatgtg

SEQ ID NO: 446 - DNA, 20 bp, artificial
  human mitoNEET antisense
tttttcttgatgatcagagg

SEQ ID NO: 447 - DNA, 20 bp, artificial
  human mitoNEET antisense
tttgcagcatcaaaagtgtc

SEQ ID NO: 448 - DNA, 20 bp, artificial
  human mitoNEET antisense
cataaaatcttttgtaagct

SEQ ID NO: 449 - DNA, 20 bp, artificial
  human mitoNEET antisense
tgaaggtttatcatagcttt

SEQ ID NO: 450 - DNA, 20 bp, artificial
  human mitoNEET antisense
gtaagctagataaccaattg

SEQ ID NO: 451 - DNA, 20 bp, artificial
  human mitoNEET antisense
atgggaactttttggacctc

SEQ ID NO: 452 - DNA, 20 bp, artificial
  human mitoNEET antisense
tccatttaagtttctttttt

SEQ ID NO: 453 - DNA, 20 bp, artificial
  human mitoNEET antisense
cagcatcaaaagtgtccatt

SEQ ID NO: 454 - DNA, 20 bp, artificial
  human mitoNEET antisense
ggcgccgtcgcttgcaaagg

SEQ ID NO: 455 - DNA, 20 bp, artificial
  human mitoNEET antisense
ggaagtcagactcatggcgc

SEQ ID NO: 456 - DNA, 20 bp, artificial
  human mitoNEET antisense
aatgtgaaagctgcagtttg

SEQ ID NO: 457 - DNA, 20 bp, artificial
  human mitoNEET antisense
tttttttgtttaaacaaatg

SEQ ID NO: 458 - DNA, 20 bp, artificial
  human mitoNEET antisense
cggcgtactaaagcaccgac

SEQ ID NO: 459 - DNA, 20 bp, artificial
  human mitoNEET antisense
ttttggacctccaacaacgg

SEQ ID NO: 460 - DNA, 20 bp, artificial
  human mitoNEET antisense
tgttttgtgtgagccccatc

SEQ ID NO: 461 - DNA, 20 bp, artificial
  human mitoNEET antisense
gacaaatgccataaatgtga

SEQ ID NO: 462 - DNA, 20 bp, artificial
  human mitoNEET antisense
cgtactaaagcaccgactcc

SEQ ID NO: 463 - DNA, 20 bp, artificial
  human mitoNEET antisense
aactggaagtcagactcatg

SEQ ID NO: 464 - DNA, 20 bp, artificial
  human mitoNEET antisense
tcatagctttatttcgatga

SEQ ID NO: 465 - DNA, 20 bp, artificial
  human mitoNEET antisense
tcacagaatgggaacttttt

SEQ ID NO: 466 - DNA, 20 bp, artificial
  human mitoNEET antisense
aatggtaactgctgcgatcc

SEQ ID NO: 467 - DNA, 20 bp, artificial
  human mitoNEET antisense
tttggacctccaacaacggc

SEQ ID NO: 468 - DNA, 20 bp, artificial
  human mitoNEET antisense
tgcagtttgcaatataccac

SEQ ID NO: 469 - DNA, 20 bp, artificial
  human mitoNEET antisense
tggatgtgaaggtttatcat

SEQ ID NO: 470 - DNA, 20 bp, artificial
  human mitoNEET antisense
atgtactatcttggggttgt

SEQ ID NO: 471 - DNA, 20 bp, artificial
  human mitoNEET antisense
agtttcttttttcttgatga

SEQ ID NO: 472 - DNA, 20 bp, artificial
  human mitoNEET antisense
ttggggttgtctttctggat

SEQ ID NO: 473 - DNA, 20 bp, artificial
  human mitoNEET antisense
gttatgttttgtgtgagccc

SEQ ID NO: 474 - DNA, 20 bp, artificial
  human mitoNEET antisense
gacaagctgatttgcagcat

SEQ ID NO: 475 - DNA, 20 bp, artificial
  human mitoNEET antisense
tttttttttgtttaaacaaa

SEQ ID NO: 476 - DNA, 20 bp, artificial
  human mitoNEET antisense
ctcatggcgccgtcgcttgc

SEQ ID NO: 477 - DNA, 20 bp, artificial
  human mitoNEET antisense
ggggttgtctttctggatgt

SEQ ID NO: 478 - DNA, 20 bp, artificial
  human mitoNEET antisense
tcatggcgccgtcgcttgca

SEQ ID NO: 479 - DNA, 20 bp, artificial
  human mitoNEET antisense
ataaccaattgcagctgtcc

SEQ ID NO: 480 - DNA, 20 bp, artificial
  human mitoNEET antisense
ttgcagcatcaaaagtgtcc

SEQ ID NO: 481 - DNA, 20 bp, artificial
  human mitoNEET antisense
ctgatttgcagcatcaaaag

SEQ ID NO: 482 - DNA, 20 bp, artificial
  human mitoNEET antisense
cttcacgacaagctgatttg

SEQ ID NO: 483 - DNA, 20 bp, artificial
  human mitoNEET antisense
catagctttatttcgatgat

SEQ ID NO: 484 - DNA, 20 bp, artificial
  human mitoNEET antisense
gtttatcatagctttatttc

SEQ ID NO: 485 - DNA, 20 bp, artificial
  human mitoNEET antisense
ctggatgtgaaggtttatca

SEQ ID NO: 486 - DNA, 20 bp, artificial
  human mitoNEET antisense
gttttgtgtgagccccatca

SEQ ID NO: 487 - DNA, 20 bp, artificial
  human mitoNEET antisense
aagtgtccatttaagtttct

SEQ ID NO: 488 - DNA, 20 bp, artificial
  human mitoNEET antisense
tgtactatcttggggttgtc

SEQ ID NO: 489 - DNA, 20 bp, artificial
  human mitoNEET antisense
atgatcagagggcccacatt

SEQ ID NO: 490 - DNA, 20 bp, artificial
  human mitoNEET antisense
ggcgtactaaagcaccgact

SEQ ID NO: 491 - DNA, 20 bp, artificial
  human mitoNEET antisense
taaaatcttttgtaagctag

SEQ ID NO: 492 - DNA, 20 bp, artificial
  human mitoNEET antisense
gcagcatcaaaagtgtccat

SEQ ID NO: 493 - DNA, 20 bp, artificial
  human mitoNEET antisense
cacgacaagctgatttgcag

SEQ ID NO: 494 - DNA, 20 bp, artificial
  human mitoNEET antisense
acttcacgacaagctgattt

SEQ ID NO: 495 - DNA, 20 bp, artificial
  human mitoNEET antisense
caagctgatttgcagcatca

SEQ ID NO: 496 - DNA, 20 bp, artificial
  human mitoNEET antisense
cagcaatggtaactgctgcg

SEQ ID NO: 497 - DNA, 20 bp, artificial
  human mitoNEET antisense
attgcagctgtcccagcagc

SEQ ID NO: 498 - DNA, 20 bp, artificial
  human mitoNEET antisense
ggatgtgaaggtttatcata

SEQ ID NO: 499 - DNA, 20 bp, artificial
  human mitoNEET antisense
ccatttaagtttcttttttc

SEQ ID NO: 500 - DNA, 20 bp, artificial
  human mitoNEET antisense
ttttttttgtttaaacaaat

SEQ ID NO: 501 - DNA, 20 bp, artificial
  human mitoNEET antisense
tggggttgtctttctggatg

SEQ ID NO: 502 - DNA, 20 bp, artificial
  human mitoNEET antisense
cagaatgggaactttttgga

SEQ ID NO: 503 - DNA, 20 bp, artificial
  human mitoNEET antisense
atcaggtaacttcacgacaa

SEQ ID NO: 504 - DNA, 20 bp, artificial
  human mitoNEET antisense
gtgccagcggcgtactaaag

SEQ ID NO: 505 - DNA, 20 bp, artificial
  human mitoNEET antisense
cgcttgcaaaggcgtgtgca

SEQ ID NO: 506 - DNA, 20 bp, artificial
  human mitoNEET antisense
tttgtgtgagccccatcaca

SEQ ID NO: 507 - DNA, 20 bp, artificial
  human mitoNEET antisense
actggaagtcagactcatgg

SEQ ID NO: 508 - DNA, 20 bp, artificial
  human mitoNEET antisense
ttgcagctgtcccagcagca

SEQ ID NO: 509 - DNA, 20 bp, artificial
  human mitoNEET antisense
gtgaaggtttatcatagctt

SEQ ID NO: 510 - DNA, 20 bp, artificial
  human mitoNEET antisense
tgctgcgatccattcaactc

SEQ ID NO: 511 - DNA, 20 bp, artificial
  human mitoNEET antisense
acagaatgggaactttttgg

SEQ ID NO: 512 - DNA, 20 bp, artificial
  human mitoNEET antisense
tcaggtaacttcacgacaag

SEQ ID NO: 513 - DNA, 20 bp, artificial
  human mitoNEET antisense
aaatgtgaaagctgcagttt

SEQ ID NO: 514 - DNA, 20 bp, artificial
  human mitoNEET antisense
gcggcgtactaaagcaccga

SEQ ID NO: 515 - DNA, 20 bp, artificial
  human mitoNEET antisense
ttgcaaaggcgtgtgcacta

SEQ ID NO: 516 - DNA, 20 bp, artificial
  human mitoNEET antisense
taactgctgcgatccattca

SEQ ID NO: 517 - DNA, 20 bp, artificial
  human mitoNEET antisense
aaaatcttttgtaagctaga

SEQ ID NO: 518 - DNA, 20 bp, artificial
  human mitoNEET antisense
ctttttggacctccaacaac

SEQ ID NO: 519 - DNA, 20 bp, artificial
  human mitoNEET antisense
gatcagagggcccacattgt

SEQ ID NO: 520 - DNA, 20 bp, artificial
  human mitoNEET antisense
ttttcttgatgatcagaggg

SEQ ID NO: 521 - DNA, 20 bp, artificial
  human mitoNEET antisense
caaaagtgtccatttaagtt

SEQ ID NO: 522 - DNA, 20 bp, artificial
  human mitoNEET antisense
cgacaagctgatttgcagca

SEQ ID NO: 523 - DNA, 20 bp, artificial
  human mitoNEET antisense
gataaccaattgcagctgtc

SEQ ID NO: 524 - DNA, 20 bp, artificial
  human mitoNEET antisense
aatgggaactttttggacct

SEQ ID NO: 525 - DNA, 20 bp, artificial
  human mitoNEET antisense
gtgtgagccccatcacagaa

SEQ ID NO: 526 - DNA, 20 bp, artificial
  human mitoNEET antisense
aacttcacgacaagctgatt

SEQ ID NO: 527 - DNA, 20 bp, artificial
  human mitoNEET antisense
gcgtactaaagcaccgactc

SEQ ID NO: 528 - DNA, 20 bp, artificial
  human mitoNEET antisense
actgctgcgatccattcaac

SEQ ID NO: 529 - DNA, 20 bp, artificial
  human mitoNEET antisense
tacacagctttatctcccaa

SEQ ID NO: 530 - DNA, 20 bp, artificial
  human mitoNEET antisense
cacagaatgggaactttttg

SEQ ID NO: 531 - DNA, 20 bp, artificial
  human mitoNEET antisense
aagctgatttgcagcatcaa

SEQ ID NO: 532 - DNA, 20 bp, artificial
  human mitoNEET antisense
tggcgccgtcgcttgcaaag

SEQ ID NO: 533 - DNA, 20 bp, artificial
  human mitoNEET antisense
tgcagctgtcccagcagcaa

SEQ ID NO: 534 - DNA, 20 bp, artificial
  human mitoNEET antisense
catggcgccgtcgcttgcaa

SEQ ID NO: 535 - DNA, 20 bp, artificial
  human mitoNEET antisense
catcaaaagtgtccatttaa

SEQ ID NO: 536 - DNA, 20 bp, artificial
  human mitoNEET antisense
cgtcgcttgcaaaggcgtgt

SEQ ID NO: 537 - DNA, 20 bp, artificial
  human mitoNEET antisense
agctgatttgcagcatcaaa

SEQ ID NO: 538 - DNA, 20 bp, artificial
  human mitoNEET antisense
taagctagataaccaattgc

SEQ ID NO: 539 - DNA, 20 bp, artificial
  human mitoNEET antisense
aaaagtgtccatttaagttt

SEQ ID NO: 540 - DNA, 20 bp, artificial
  human mitoNEET antisense
tgtttcaacaagacaaatgc

SEQ ID NO: 541 - DNA, 20 bp, artificial
  human mitoNEET antisense
atgtttcaacaagacaaatg

SEQ ID NO: 542 - DNA, 20 bp, artificial
  human mitoNEET antisense
gtctttctggatgtgaaggt

SEQ ID NO: 543 - DNA, 20 bp, artificial
  human mitoNEET antisense
atggcgccgtcgcttgcaaa

SEQ ID NO: 544 - DNA, 20 bp, artificial
  human mitoNEET antisense
agccccatcacagaatggga

SEQ ID NO: 545 - DNA, 20 bp, artificial
  human mitoNEET antisense
ttcttgatgatcagagggcc

SEQ ID NO: 546 - DNA, 20 bp, artificial
  human mitoNEET antisense
caggtaacttcacgacaagc

SEQ ID NO: 547 - DNA, 20 bp, artificial
  human mitoNEET antisense
accaattgcagctgtcccag

SEQ ID NO: 548 - DNA, 20 bp, artificial
  human mitoNEET antisense
actttttggacctccaacaa

SEQ ID NO: 549 - DNA, 20 bp, artificial
  human mitoNEET antisense
tgtgagccccatcacagaat

SEQ ID NO: 550 - DNA, 20 bp, artificial
  human mitoNEET antisense
gctgatttgcagcatcaaaa

SEQ ID NO: 551 - DNA, 20 bp, artificial
  human mitoNEET antisense
aactgctgcgatccattcaa

SEQ ID NO: 552 - DNA, 20 bp, artificial
  human mitoNEET antisense
taaatgtgaaagctgcagtt

SEQ ID NO: 553 - DNA, 20 bp, artificial
  human mitoNEET antisense
ctgctgcgatccattcaact

SEQ ID NO: 554 - DNA, 20 bp, artificial
  human mitoNEET antisense
tttcttgatgatcagagggc

SEQ ID NO: 555 - DNA, 20 bp, artificial
  human mitoNEET antisense
agctgtcccagcagcaatgg

SEQ ID NO: 556 - DNA, 20 bp, artificial
  human mitoNEET antisense
tcaaaagtgtccatttaagt

SEQ ID NO: 557 - DNA, 20 bp, artificial
  human mitoNEET antisense
acaagctgatttgcagcatc

SEQ ID NO: 558 - DNA, 20 bp, artificial
  human mitoNEET antisense
agcggcgtactaaagcaccg

SEQ ID NO: 559 - DNA, 20 bp, artificial
  human mitoNEET antisense
tcgcttgcaaaggcgtgtgc

SEQ ID NO: 560 - DNA, 20 bp, artificial
  human mitoNEET antisense
tagataaccaattgcagctg

SEQ ID NO: 561 - DNA, 20 bp, artificial
  human mitoNEET antisense
ctagataaccaattgcagct

SEQ ID NO: 562 - DNA, 20 bp, artificial
  human mitoNEET antisense
gatgtgaaggtttatcatag

SEQ ID NO: 563 - DNA, 20 bp, artificial
  human mitoNEET antisense
gaatgggaactttttggacc

SEQ ID NO: 564 - DNA, 20 bp, artificial
  human mitoNEET antisense
atcaaaagtgtccatttaag

SEQ ID NO: 565 - DNA, 20 bp, artificial
  human mitoNEET antisense
tgccagcggcgtactaaagc

SEQ ID NO: 566 - DNA, 20 bp, artificial
  human mitoNEET antisense
acgacaagctgatttgcagc

SEQ ID NO: 567 - DNA, 20 bp, artificial
  human mitoNEET antisense
aggtaacttcacgacaagct

SEQ ID NO: 568 - DNA, 20 bp, artificial
  human mitoNEET antisense
cagcggcgtactaaagcacc

SEQ ID NO: 569 - DNA, 20 bp, artificial
  human mitoNEET antisense
gccagcggcgtactaaagca

SEQ ID NO: 570 - DNA, 20 bp, artificial
  human mitoNEET antisense
aaatcttttgtaagctagat

SEQ ID NO: 571 - DNA, 20 bp, artificial
  human mitoNEET antisense
aatcttttgtaagctagata

SEQ ID NO: 572 - DNA, 20 bp, artificial
  human mitoNEET antisense
cccatcacagaatgggaact

SEQ ID NO: 573 - DNA, 20 bp, artificial
  human mitoNEET antisense
gccccatcacagaatgggaa

SEQ ID NO: 574 - DNA, 20 bp, artificial
  human mitoNEET antisense
atcttttgtaagctagataa

SEQ ID NO: 575 - DNA, 20 bp, artificial
  human mitoNEET antisense
gagccccatcacagaatggg

SEQ ID NO: 576 - DNA, 20 bp, artificial
  human mitoNEET antisense
ccaattgcagctgtcccagc

SEQ ID NO: 577 - DNA, 20 bp, artificial
  human mitoNEET antisense
ataaatgtgaaagctgcagt

SEQ ID NO: 578 - DNA, 20 bp, artificial
  human mitoNEET antisense
aagacaaatgccataaatgt

SEQ ID NO: 579 - DNA, 20 bp, artificial
  human mitoNEET antisense
aagctagataaccaattgca

SEQ ID NO: 580 - DNA, 20 bp, artificial
  human mitoNEET antisense
ttgtgtgagccccatcacag

SEQ ID NO: 581 - DNA, 20 bp, artificial
  human mitoNEET antisense
tgtgtgagccccatcacaga

SEQ ID NO: 582 - DNA, 20 bp, artificial
  human mitoNEET antisense
caattgcagctgtcccagca

SEQ ID NO: 583 - DNA, 20 bp, artificial
  human mitoNEET antisense
tttcaacaagacaaatgcca

SEQ ID NO: 584 - DNA, 20 bp, artificial
  human mitoNEET antisense
cagctgtcccagcagcaatg

SEQ ID NO: 585 - DNA, 20 bp, artificial
  human mitoNEET antisense
ttcaacaagacaaatgccat

SEQ ID NO: 586 - DNA, 20 bp, artificial
  human mitoNEET antisense
tgatcagagggcccacattg

SEQ ID NO: 587 - DNA, 20 bp, artificial
  human mitoNEET antisense
ccagcggcgtactaaagcac

SEQ ID NO: 588 - DNA, 20 bp, artificial
  human mitoNEET antisense
ccccatcacagaatgggaac

SEQ ID NO: 589 - DNA, 20 bp, artificial
  human mitoNEET antisense
tgagccccatcacagaatgg

SEQ ID NO: 590 - DNA, 20 bp, artificial
  human mitoNEET antisense
aattgcagctgtcccagcag

SEQ ID NO: 591 - DNA, 20 bp, artificial
  human mitoNEET antisense
tcttttgtaagctagataac

SEQ ID NO: 592 - DNA, 20 bp, artificial
  human mitoNEET antisense
atgtgaaggtttatcatagc

SEQ ID NO: 593 - DNA, 20 bp, artificial
  human mitoNEET antisense
gtttcaacaagacaaatgcc

SEQ ID NO: 594 - DNA, 20 bp, artificial
  human mitoNEET antisense
caaatgccataaatgtgaaa

SEQ ID NO: 595 - DNA, 20 bp, artificial
  human mitoNEET antisense
acaaatgccataaatgtgaa

SEQ ID NO: 596 - DNA, 20 bp, artificial
  human mitoNEET antisense
tcaacaagacaaatgccata

SEQ ID NO: 597 - DNA, 20 bp, artificial
  human mitoNEET antisense
gtcgcttgcaaaggcgtgtg

SEQ ID NO: 598 - DNA, 20 bp, artificial
  human mitoNEET antisense
cataaatgtgaaagctgcag

SEQ ID NO: 599 - DNA, 20 bp, artificial
  human mitoNEET antisense
caacaagacaaatgccataa

SEQ ID NO: 600 - DNA, 20 bp, artificial
  human mitoNEET antisense
aaatgccataaatgtgaaag

SEQ ID NO: 601 - DNA, 20 bp, artificial
  human mitoNEET antisense
agacaaatgccataaatgtg

SEQ ID NO: 602 - DNA, 20 bp, artificial
  human mitoNEET antisense
tcttgatgatcagagggccc

SEQ ID NO: 603 - DNA, 20 bp, artificial
  human mitoNEET antisense
gcagctgtcccagcagcaat

SEQ ID NO: 604 - DNA, 20 bp, artificial
  human mitoNEET antisense
ccataaatgtgaaagctgca

SEQ ID NO: 605 - DNA, 20 bp, artificial
  human mitoNEET antisense
aatgccataaatgtgaaagc

SEQ ID NO: 606 - DNA, 20 bp, artificial
  human mitoNEET antisense
cttgatgatcagagggccca

SEQ ID NO: 607 - DNA, 20 bp, artificial
  human mitoNEET antisense
caagacaaatgccataaatg

SEQ ID NO: 608 - DNA, 20 bp, artificial
  human mitoNEET antisense
ttgatgatcagagggcccac

SEQ ID NO: 609 - DNA, 20 bp, artificial
  human mitoNEET antisense
atgccataaatgtgaaagct

SEQ ID NO: 610 - DNA, 20 bp, artificial
  human mitoNEET antisense
acaagacaaatgccataaat

SEQ ID NO: 611 - DNA, 20 bp, artificial
  human mitoNEET antisense
tgtgaaggtttatcatagct

SEQ ID NO: 612 - DNA, 20 bp, artificial
  human mitoNEET antisense
aacaagacaaatgccataaa

SEQ ID NO: 613 - DNA, 20 bp, artificial
  human mitoNEET antisense
gtgagccccatcacagaatg

SEQ ID NO: 614 - DNA, 20 bp, artificial
  human mitoNEET antisense
tgccataaatgtgaaagctg

SEQ ID NO: 615 - DNA, 20 bp, artificial
  human mitoNEET antisense
gctagataaccaattgcagc

SEQ ID NO: 616 - DNA, 20 bp, artificial
  human mitoNEET antisense
agctagataaccaattgcag

SEQ ID NO: 617 - DNA, 20 bp, artificial
  human mitoNEET antisense
gccataaatgtgaaagctgc

SEQ ID NO: 618 - DNA, 20 bp, Artificial
  human mitoNEET forward PCR primer
tcctagtgcacacgcctttg

SEQ ID NO: 619 - DNA, 21 bp, Artificial
  human mitoNEET reverse PCR primer
actcgtacgctggaactggaa

SEQ ID NO: 620 - DNA, 22 bp, Artificial
  human mitoNEET PCR probe
aagcgacggcgccatgagtctg

SEQ ID NO: 621 - DNA, 20 bp, Artificial
  human cyclophilin forward PCR primer
cccaccgtgttcttcgacat

SEQ ID NO: 622 - DNA, 22 bp, Artificial
  human cyclophilin reverse PCR primer
tttctgctgtctttgggacctt

SEQ ID NO: 623 - DNA, 24 bp, Artificial
  human cyclophilin PCR probe
cgcgtctcctttgagctgtttgca

SEQ ID NO: 624 - DNA, 636 bp, Homo sapiens
gatcgcggagtcggtgctttagtacgccgctggcacctttactctcgccggccgcgcgaacccgtttgagctcggtatcctagtgcacacgcctttgcaagcgacggcgccatgagtctgacttccagttccagcgtacgagttgaatggatcgcagcagttaccattgctgctgggacagctgcaattggttatctagcttacaaaagattttatgttaaagatcatcgaaataaagctatgataaaccttcacatccagaaagacaaccccaagatagtacatgcttttgacatggaggatttgggagataaagctgtgtactgccgttgttggaggtccaaaaagttcccattctgtgatggggctcacacaaaacataacgaagagactggagacaatgtgggccctctgatcatcaagaaaaaagaaacttaaatggacacttttgatgctgcaaatcagcttgtcgtgaagttacctgattgtttaattagaatgactaccacctctgtctgattcaccttcgctggattctaaatgtggtatattgcaaactgcagctttcacatttatggcatttgtcttgttgaaacatcgtggtgcacatttgtttaaacaaaaaaaaaaaaaaaa

SEQ ID NO: 625 - protein, 108 aa, Homo sapiens
MSLTSSSSVRVEWIAAVTIAAGTAAIGYLAYKRFYVKDHRNKAMINLHIQKDNPKIVHAFDMEDLGDKAVYCRCWRSKKFPFCDGAHTKHNEETGDNVGPLIIKKKET

SEQ ID NO: 626 - protein, 106 aa, Bos taurus
MSMTSSVRVEWIAAVTIAAGTAAIGYLAYKRFYVKDHRNKSMINPHIQKDNPKVVHAFDMEDLGDKAVYCRCWRSKKFPLCDGSHTKHNEETGDNVGPLIIKKKDT

SEQ ID NO: 627 - protein, 108 aa, Mus musculus
MGLSSNSAVRVEWIAAVTFAAGTAALGYLAYKKFYAKENRTKAMVNLQIQKDNPKVVHAFDMEDLGDKAVYCRCWRSKKFPFCDGAHIKHNEETGDNVGPLIIKKKET

(19) United States
(12) Patent Application Publication
(10) Pub. No.: US 2004/0132078 A1
(43) Pub. Date: Jul. 8, 2004

(54) Antisense modulation of mitoNEET expression

(76) Inventor: Jerry R. Colca, Chesterfield, MO (US)

Correspondence Address: Pharmacia Corporation, Global Patent Department, P.O. Box 1027, Mail Zone MC5, St. Louis, MO, 63141, US

(21) Appl. No.: 10/728,399

(22) Filed: Dec. 5, 2003

Related U.S. Application Data
(60) Provisional application No. 60/431,529, filed on Dec. 6, 2002.

(51) Int. Cl. C12Q 1/68; C07H 21/04; A61K 48/00

(52) U.S. Cl. 435/6; 514/44; 536/23.2

(57) ABSTRACT

Antisense compounds, compositions, and methods are provided for modulating the expression of a family of polypeptides from mitochondrial membranes, which bind insulin sensitizing, antidiabetic thiazolodinediones (mitoNEET). The compositions comprise antisense compounds, particularly antisense oligonucleotides, targeted to nucleic acids encoding mitoNEET. Methods of using these compounds for modulation of mitoNEET expression and for treatment of diseases associated with expression of mitoNEET are provided.

The present application claims priority under Title 35, United States Code, § 119 to U.S. Provisional application Serial No. 60/431,529, filed Dec. 6, 2002, which is incorporated by reference in its entirety as if written herein.

### FIELD OF THE INVENTION

The present invention provides compositions and methods for modulating the expression of a family of polypeptides from mitochondrial membranes, which bind insulin sensitizing, antidiabetic thiazolodinediones (referred to as “mitoNEET”). In particular, this invention relates to antisense compounds, particularly oligonucleotides, specifically hybridizable with nucleic acids encoding mitoNEET. Such oligonucleotides have been shown to modulate the expression of mitoNEET.

### BACKGROUND OF THE INVENTION

Non-insulin-dependent diabetes mellitus (NIDDM) or Type 2 Diabetes is characterized by insulin resistance of the peripheral tissues, including the skeletal muscle, liver, and adipose. The resulting hyperglycemia is often accompanied by defective lipid metabolism that can lead to cardiovascular complications such as atherosclerosis and hypertension.

Thiazolidinediones comprise a group of structurally related antidiabetic compounds that increases the insulin sensitivity of target tissues (skeletal muscle, liver, adipose) in insulin resistant animals. In addition to these effects on hyperglycemia, thiazolidinediones also reduce lipid and insulin levels in animal models of NIDDM. The thiazolidinediones troglitazone, rosiglitazone, and pioglitazone have been shown to have these same beneficial effects in human patients suffering from impaired glucose tolerance, a metabolic condition that precedes the development of NIDDM, as in patients suffering from NIDDM (e.g., Nolan et al., N. Eng. J. Med. 331, 1188-1193, 1994). While their mechanism of action remains unclear, it is known that the thiazolidinediones do not cause increases in insulin secretion or in the number or affinity of insulin receptor binding sites, suggesting that thiazolidinediones amplify post-receptor events in the insulin signaling cascade (Colca and Morton, New Antidiabetic Drugs (C. J. Bailey and P. R. Flatt, eds.). Smith-Gordon, New York, 255-261, 1990, Chang et al., Diabetes 32: 839-845, 1983).

Thiazolidinediones have been found to be efficacious inducers of differentiation in cultured pre-adipocyte cell lines (Hiragun et al., J. Cell Physiol. 134:124-130, 1988; Sparks et al., J. Cell. Physiol. 146:101-109, 1991; Kletzien et al., Mol. Pharmacol. 41:393-398, 1992). Treatment of pre-adipocyte cell lines with the thiazolidinedione pioglitazone results in increased expression of the adipocyte-specific genes aP2 and adipsin as well as the glucose transporter proteins GLUT-I and GLUT-4. These data suggest that the hypoglycemic effects of thiazolidinediones seen in vivo may be mediated through adipose tissue. However, as estimates of the contribution of adipose tissue to whole body glucose usage range from only 1-3%, it remains unclear whether the hypoglycemic effects of thiazolidinediones can be accounted for by changes in adipocytes. Additionally, thiazolidinediones have been implicated in appetite regulation disorders, see PCT patent application WO 94/25026 A1, and in increase of bone marrow fat content, (Williams, et al, Diabetes 42, Supplement 1, p. 59A1993).

Peroxisome proliferator-activated receptor γ (PPARγ) is an orphan member of the steroid/thyroid/retinoid superfamily of ligand-activated transcription factors. PPARγ is one of a subfamily of closely related PPARs encoded by independent genes (Dreyer et al., Cell 68:879-887, 1992; Schmidt et al, J. Cell. Physiol. 146:101-1091992; Zhu et al., J. Biol. Chem. 268:26817-26820, 1993; Kliewer et al., Proc. Natl. Acad. Sci. USA 91:7355-7359, 1994). Three mammalian PPARs have been identified and termed PPARα, γ, and NUC-1. Homologs of PPARα and γ have been identified in the frog, Xenopus laevis; however, a third Xenopus PPAR, termed PPARβ, is not a NUC-1 homolog, leading to the suggestion that there may be additional subtypes in either or both species.

The PPARs are activated to various degrees by high (micromolar) concentrations of long-chain fatty acids and peroxisome proliferators (Isseman and Green, Nature 347, 645-650, 1990; Gottlicher, Proc. Natl. Acad. USA 89, 4653-4657, 1992). Peroxisome proliferators are a structurally diverse group of compounds that includes herbicides, phthalate plasticizers, and the fibrate class of hypolipidemic drugs. While these data suggest that the PPARs are bona fide receptors, they remain “orphans” as none of these compounds have been shown to interact directly with the PPARs.

PPARs regulate expression of target genes by binding to DNA sequence elements, termed PPAR response elements (PPRE), as heterodimers with the retinoid X receptors (reviewed in Keller and Whali, Trends Endocrin. Met. 4:291-296, 1993). To date, PPREs have been identified in the enhancers of a number of genes encoding proteins that regulate lipid metabolism including the three enzymes required for peroxisomal beta-oxidation of fatty acids, medium-chain acyl-CoA dehydrogenase, a key enzyme in mitochondrial beta-oxidation, and aP2, a lipid binding protein expressed exclusively in adipocytes. The nature of the PPAR target genes coupled with the activation of PPARs by fatty acids and hypolipidemic drugs suggests a physiological role for the PPARs in lipid homeostasis (reviewed in Keller and Whali, Trends Endocrin. Met. 4:291-296, 1993).

A second isoform of PPARγ, termed PPARγ2, was cloned from a mouse adipocyte library (Tontonoz et al., Genes & Dev. 8, 1224-1234, 1994). PPARγ1 and γ2 differ in only 30 amino acids at the extreme N-terminus of the receptor and likely arise from a single gene. PPARγ 2 is expressed in a strikingly adipose-specific manner and its expression is markedly induced during the course of differentiation of several preadipocyte cell lines; furthermore, forced expression of PPARγ2 was shown to be sufficient to activate the adipocyte-specific aP2 enhancer in non-adipocyte cell lines. These data suggest that PPARγ2 plays an important role in adipocyte differentiation.

The thiazolidinedione pioglitazone was reported to stimulate expression of a chimeric gene containing the enhancer/promoter of the lipid-binding protein aP2 upstream of the chloroamphenicol acetyl transferase reporter gene (Harris and Kletzien, Mol. Pharmacol. 45:439-445, 1994). Deletion analysis led to the identification of an approximately 30 bp region responsible for pioglitazone responsiveness. Interestingly, in an independent study, this 30 bp fragment was shown to contain a PPRE (Tontonoz et al., Genes & Dev. 8:1224-1234, 1994). Taken together, these studies suggested the possibility that the thiazolidinediones modulate gene expression at the transcriptional level through interactions with a PPAR.

Insulin-sensitizing thiazolidinedione have shown efficacy as potential anti-cancer agents in breast cancer, colon cancer, pancreatic cancer, and hepatoma (e.g. Mueller, E. et al.,. Molecular Cell (1998), 1(3), 465-470; Tanaka, T. et al., Cancer Research (2001), 61(6), 2424-2428; Itami, A. et al., International Journal of Cancer (2001), 94(3), 370-376; Goeke, R. et al., Digestion (2001), 64(2), 75-80; Okano, H et al., Anti-Cancer Drugs (2002), 13(1), 59-65; and WO/0243716).

Current evidence suggests that a simple direct interaction with nuclear receptors may not explain the pharmacology of these promising drugs. Efforts to improve on the pharmacology by directly targeting PPAR nuclear receptors have not yet proven successful. It is possible that an additional site of action may be relevant. We have shown that thiazoldinediones also bind directly to mitochondria and used a photoaffinity probe to label a 17-kDa protein, referred to as “mitoNEET”, as the potential target for this interaction.

Homologous amino acid and nucleic sequences of a human polypeptide described as an uncharacterized hematopoietic stem/progenitor cell protein (MDS029) are disclosed (Genbank Accession Number NM_{—}018464).

Homologous amino acid and nucleic sequences of an uncharacterized murine polypeptide are disclosed (Genbank Accession Number NM_{—}134007).

Antisense technology is emerging as an effective means for reducing the expression of specific gene products and may therefore prove to be uniquely useful in a number of therapeutic, diagnostic, and research applications for the modulation of mitoNEET expression.

### SUMMARY OF THE INVENTION

The present invention is directed to antisense compounds, particularly oligonucleotides, which are targeted to a nucleic acid encoding mitoNEET, and which modulate the expression of mitoNEET. Pharmaceutical and other compositions comprising the antisense compounds of the invention are also provided. Further provided are methods of modulating the expression of mitoNEET in cells or tissues comprising contacting said cells or tissues with one or more of the antisense compounds or compositions of the invention. Further provided are methods of treating an animal, particularly a human, suspected of having or being prone to a disease or condition associated with expression of mitoNEET by administering a therapeutically or prophylactically effective amount of one or more of the antisense compounds or compositions of the invention.

### BRIEF DESCRIPTION OF THE FIGURE

FIG. 1. Amino acid sequence of human mitoNEET and a nucleic acid encoding same.

FIG. 2. Sequence alignment of bovine, human, and murine mitoNEET.

FIG. 3. Underlined amino sequence was found experimentally by nanospray mass spectral analysis of tryptic peptides from purified mitoNEET. Sequence after M62 was confirmed by N-terminal sequencing of purified CnBr fragment. This portion of the molecule contains the site of crosslinking by the probe.

### DETAILED DESCRIPTION OF THE INVENTION

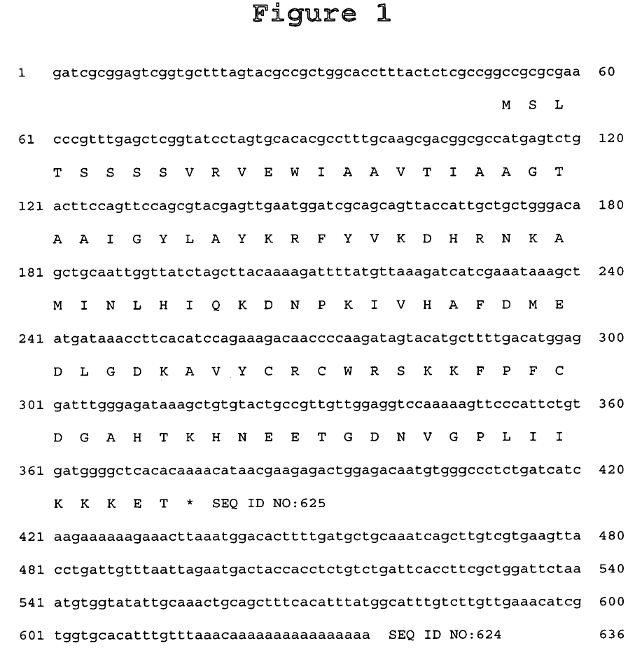

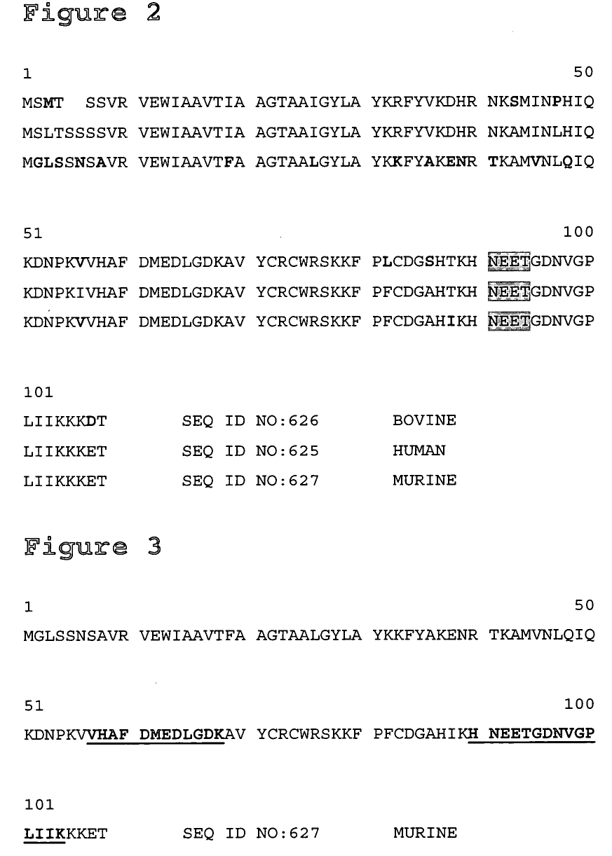

It is well known in the art that antidiabetic thiazolidinediones, some of which have recently been approved for use in man, are insulin sensitizers (Hofmann CA. et al., Diabetes Care. 15(8):1075-8, 1992; Goldstein B J., Rosiglitazone. International Journal of Clinical Practice. 54(5):333-7, 2000; Lawrence J M. et al., Pioglitazone. International Journal of Clinical Practice. 54(9):614-8, 2000). Further it is well accepted that the molecular mechanism of action of these compounds involves direct interaction/modulation of the nuclear receptor, PPARγ (Olefsky J M. et al., Trends in Endocrinology & Metabolism. 11(9):362-8, 2000; Lenhard J M. Receptors & Channels. 7(4):249-58, 2001; Lehmann J M. et al., Journal of Biological Chemistry. 270(22):12953-6, 1995). Thus, there have been a plethora of attempts by those well-schooled in the art to find other compounds that are better modulators of this and other similar nuclear receptors to produce better therapeutic agents for treatment of metabolic disease (Willson T M. Et al., Annals of the New York Academy of Sciences. 804:276-83, 1996; Henke B R. Et al., Bioorganic & Medicinal Chemistry Letters. 9(23):3329-34, 1999; Murakami K. et al., Diabetes. 47(12):1841-7, 1998; Cesario R M. Et al., Molecular Endocrinology. 15(8):1360-9, 2001; Elbrecht A. et al., Journal of Biological Chemistry. 274(12):7913-22, 1999; Brown K K. Et al., Diabetes. 48(7):1415-24, 1999). Because of the effects of these antidiabetic compounds on inhibition of apoptosis and inflammation, compounds of this class are expected to have relevance in the control of cancers as well diseases related to neurodegeneration and inflammation (Eibl G. et al., Biochemical & Biophysical Research Communications. 287(2):522-9, 2001; Takashima T. et al., International Journal of Oncology. 19(3):465-71, 2001; Goke R. et al., Digestion. 64(2):75-80, 2001; Rohn T T. Et al., Neuroreport. 12(4):839-43, 2001; Patel L. et al., Current Biology. 11(10):764-8, 2001). It is generally accepted that all of these effects occur secondary to direct modulation of nuclear receptors, especially PPARγ. On the other hand, we have discovered that prototypical thiazolidinediones bind to mitochondria and we have developed a novel photoprobe that we have used to locate the site of specific binding to a <17 kDa mitochondrial protein. We have identified this protein by biochemical separation techniques and have obtained its amino acid sequence by both mass spectral techniques and N-terminal sequence of a CnBr fragment containing the residues that are crosslinked by radiolabeled photoaffinity probe. This sequence exists previously in the public domain only as predicted from DNA sequence from both the mouse and human genome and from expressed sequence tags found an in house library from bovine kidney. The expected sequences of the protein in these three species are shown in FIG. 1. The sequences of tryptic fragments of the protein that we have determined experimentally by nanospray mass spectroscopy of purified, photoprobe crosslinked protein from bovine brain mitochondria and rat liver mitochondria are shown in FIG. 2. These peptide sequences agree with N-terminal sequencing of the CnBr fragmentation of our crosslinked bovine brain mitochondrial protein starting at the methionine (M) found at position 62. The key components of our discovery are that this actual protein exists in the mitochondria (two tissues from two species) and that the protein is unexpectedly involved in the direct recognition of insulin sensitizer molecules. Our discovery will allow the use of this protein and factors involved in regulating its expression and disposition to discover novel therapeutics and therapeutic strategies.

The present invention employs oligomeric antisense compounds, particularly oligonucleotides, for use in modulating the function of nucleic acid molecules encoding mitoNEET, ultimately modulating the amount of mitoNEET produced. This is accomplished by providing antisense compounds, which specifically hybridize with one or more nucleic acids encoding mitoNEET. As used herein, the terms “target nucleic acid” and “nucleic acid encoding mitoNEET” encompass DNA encoding mitoNEET, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds, which specifically hybridize to it, is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of mitoNEET. In the context of the present invention, “modulation” means either an increase (stimulation) or a decrease (inhibition) in the expression of a gene. In the context of the present invention, inhibition is the preferred form of modulation, of gene expression and mRNA is a preferred target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding mitoNEET. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding mitoNEET, regardless of the sequence(s) of such codons.

It is also known in the art that a translation termination codon (or “stop codon”) of a gene may have one of three sequences, i.e. 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases, which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans. In the context of this invention, the term “oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for nucleic acid target and increased stability in the presence of nucleases. Syndrome X (including metabolic syndrome) is loosely defined as a collection of abnormalities including hyperinsulemia, obesity, elevated levels of triglycerides, uric acid, 20 fibrinogen, small dense LDL particles, plasminogen activator inhibitor 1 (PAI-1), and decreased levels of HDL c.

Similar metabolic conditions include dyslipidemia including associated diabetic dyslipidemia and mixed dyslipidemia, syndrome X (as defined in this application this embraces metabolic syndrome), heart failure, hypercholesteremia, cardiovascular disease including atherosclerosis, arteriosclerosis, and hypertriglyceridemia, type 11 diabetes mellitus, type I diabetes, insulin resistance, hyperlipidemia, inflammation, epithelial hyperproliferative diseases 25 including eczema and psoriasis and conditions associated with the lung and gut and regulation of appetite and food intake in subjects suffering from disorders such as obesity, anorexia bulimia, and anorexia nervosa. In particular, the compounds of this invention are useful in the treatment and prevention of diabetes and cardiovascular diseases and conditions including atherosclerosis, arteriosclerosis, hypertriglyceridemia, and mixed dyslipidaemia.

MitoNEET, or modulators thereof, that has activity in the cardiovascular, angiogenic, and endothelial assays described herein, and/or whose gene product has been found to be localized to the cardiovascular system, is likely to have therapeutic uses in a variety of cardiovascular, endothelial, and angiogenic disorders, including systemic disorders that affect vessels, such as diabetes mellitus. Its therapeutic utility could include diseases of the arteries, capillaries, veins, and/or lymphatics. Examples of treatments hereunder include treating muscle wasting disease, treating osteoporosis, aiding in implant fixation to stimulate the growth of cells around the implant and therefore facilitate its attachment to its intended site, increasing IGF stability in tissues or in serum, if applicable, and increasing binding to the IGF receptor (since IGF has been shown in vitro to enhance human marrow erythroid and granulocytic progenitor cell growth).

MitoNEET or modulators thereof may also be employed to stimulate erythropoiesis or granulopoiesis, to stimulate wound healing or tissue regeneration and associated therapies concerned with re-growth of tissue, such as connective tissue, skin, bone, cartilage, muscle, lung, or kidney, to promote angiogenesis, to stimulate or inhibit migration of endothelial cells, and to proliferate the growth of vascular smooth muscle and endothelial cell production. The increase in angiogenesis mediated by mitoNEET or agonist would be beneficial to ischemic tissues and to collateral coronary development in the heart subsequent to coronary stenosis.

Antagonists are used to inhibit the action of such polypeptides, for example, to limit the production of excess connective tissue during wound healing or pulmonary fibrosis if mitoNEET promotes such production. This would include treatment of acute myocardial infarction and heart failure.

Moreover, the present invention provides the treatment of cardiac hypertrophy, regardless of the underlying cause, by administering a therapeutically effective dose of mitoNEET, or agonist or antagonist thereto.

If the objective is the treatment of human patients, mitoNEET preferably is recombinant human mitoNEET polypeptide (rhmitoNEET polypeptide). The treatment for cardiac hypertrophy can be performed at any of its various stages, which may result from a variety of diverse pathologic conditions, including myocardial infarction, hypertension, hypertrophic cardiomyopathy, and valvular regurgitation. The treatment extends to all stages of the progression of cardiac hypertrophy, with or without structural damage of the heart muscle, regardless of the underlying cardiac disorder.

The decision of whether to use the molecule itself or an agonist thereof for any particular indication, as opposed to an antagonist to the molecule, would depend mainly on whether the molecule herein promotes cardio vascularization, genesis of endothelial cells, or angiogenesis or inhibits these conditions. For example, if the molecule promotes angiogenesis, an antagonist thereof would be useful for treatment of disorders where it is desired to limit or prevent angiogenesis. Examples of such disorders include vascular tumors such as haemangioma, tumor angiogenesis, neovascularization in the retina, choroid, or cornea, associated with diabetic retinopathy or premature infant retinopathy or macular degeneration and proliferative vitreoretinopathy, rheumatoid arthritis, Crohn's disease, atherosclerosis, ovarian hyperstimulation, psoriasis, endometriosis associated with neovascularization, restenosis subsequent to balloon angioplasty, sear tissue overproduction, for example, that seen in a keloid that forms after surgery, fibrosis after myocardial infarction, or fibrotic lesions associated with pulmonary fibrosis.

If, however, the molecule inhibits angiogenesis, it would be expected to be used directly for treatment of the above conditions.

On the other hand, if the molecule stimulates angiogenesis it would be used itself (or an agonist thereof) for indications where angiogenesis is desired such as peripheral vascular disease, hypertension, inflammatory vasculitides, Reynaud's disease and Reynaud's phenomenon, aneurysms, arterial restenosis, thrombophlebitis, lymphangitis, lymphedema, wound healing and tissue repair, ischemia reperfusion injury, angina, myocardial infarctions such as acute myocardial infarctions, chronic heart conditions, heart failure such as congestive heart failure, and osteoporosis.

If, however, the molecule inhibits angiogenesis, an antagonist thereof would be used for treatment of those conditions where angiogenesis is desired.

Specific types of diseases are described below, where mitoNEET or agonists or antagonists thereof may serve as useful for vascular-related drug targeting or as therapeutic targets for the treatment or prevention of the disorders.

Atherosclerosis is a disease characterized by accumulation of plaques of intimal thickening in arteries, due to accumulation of lipids, proliferation of smooth muscle cells, and formation of fibrous tissue within the arterial wall. The disease can affect large, medium, and small arteries in any organ. Changes in endothelial and vascular smooth muscle cell function are known to play an important role in modulating the accumulation and regression of these plaques.

Hypertension is characterized by raised vascular pressure in the systemic arterial, pulmonary arterial, or portal venous systems. Elevated pressure may result from or result in impaired endothelial function and/or vascular disease.

Inflammatory vasculitides include giant cell arteritis, Takayasu's arteritis, polyarteritis nodosa (including the microangiopathic form), Kawasaki's disease, microscopic polyarightis, Wegener's granulomatosis, and a variety 101 of infectious-related vascular disorders (including Henoch-Schonlein Prupura). Altered endothelial cell function has been shown to be important in these diseases. Reynaud's disease and Reynaud's phenomenon are characterized by intermittent abnormal impairment of the circulation through the extremities on exposure to cold. Altered endothelial cell function has been shown to be important in this disease.

Aneurysms are saccular or fusiform dilatations of the arterial or venous tree that are associated with altered endothelial cell and/or vascular smooth muscle cells.

Arterial restenosis (restenosis of the arterial wall) may occur following angioplasty as a result of alteration in the function and proliferation of endothelial and vascular smooth muscle cells.

Thrombophlebitis and lymphangitis are inflammatory disorders of veins and lymphatics, respectively, that may result from, and/or in, altered endothelial cell function. Similarly, lymphedema is a condition involving impaired lymphatic vessels resulting from endothelial cell function.

The family of benign and malignant vascular tumors is characterized by abnormal proliferation and growth of cellular elements of the vascular system. For example, lymphangiomas are benign tumors of the lymphatic system that are congenital, often cystic, malformations of the lymphatics that usually occur in newborns.

Cystic tumors tend to grow into the adjacent tissue. Cystic tumors usually occur in the cervical and axillary region. They can also occur in the soft tissue of the extremities. The main symptoms are dilated, sometimes reticular, structured lymphatics and lymphocysts surrounded by connective tissue.

Lymphangiomas are assumed to be caused by improperly connected embryonic lymphatics or their deficiency. The result is impaired local lymph drainage.

Another use for mitoNEET antagonists thereto is in the prevention of tumor angiogenesis, which involves vascularization of a tumor to enable it to growth and/or metastasize. This process is dependent on the growth of new blood vessels. Examples of neoplasms and related conditions that involve tumor angiogenesis include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendrogliorna, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, mitoNEET agonist thereto is expected to be useful in reducing the severity of AMD.

Healing of trauma such as wound healing and tissue repair is also a targeted use for mitoNEET or its agonists. Formation and regression of new blood vessels is essential for tissue healing and repair. This category includes bone, cartilage, tendon, ligament, and/or nerve tissue growth or regeneration, as well as wound healing and tissue repair and replacement, and in the treatment of burns, incisions, and ulcers.

MitoNEET or modulators thereof that induces cartilage and/or bone growth in circumstances where bone is not normally formed has application in the healing of bone fractures and cartilage damage or defects in humans and other animals. Such a preparation employing mitoNEET or agonist or antagonist thereof may have prophylactic use in closed as well as open fracture reduction and also in the improved fixation of artificial joints. De novo bone formation induced by an osteogenic agent contributes to the repair of congenital, trauma induced, or oncologic, resection-induced craniofacial defects, and also is useful in cosmetic plastic surgery.

MitoNEET or modulators thereof may also be useful to promote better or faster closure of non-healing wounds, including without limitation pressure ulcers, ulcers associated with vascular insufficiency, surgical and traumatic wounds, and the like.

It is expected that mitoNEET modulators may also exhibit activity for generation or regeneration of other tissues, such as organs (including, for example, pancreas, liver, intestine, kidney, skin, or endothelium), muscle (smooth, skeletal, or cardiac), and vascular (including vascular endothelium) tissue, or for promoting the growth of cells comprising such tissues. Part of the desired effects may be by inhibition or modulation of fibrotic scarring to allow normal tissue to regenerate.

MitoNEET modulators may also be useful for gut protection or regeneration and treatment of lung or liver fibrosis, reperfusion injury in various tissues, and conditions resulting from systemic cytokine damage. Also, mitoNEET or modulators thereof may be useful for promoting or inhibiting differentiation of tissues described above from precursor tissues or cells, or for inhibiting the growth of tissues described above.

MitoNEET modulators may also be used in the treatment of periodontal diseases and in other tooth-repair processes. Such agents may provide an environment to attract bone-forming cells, stimulate growth of bone-forming cells, or induce differentiation of progenitors of bone-forming cells mitoNEET or an agonist or an antagonist thereto may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes, since blood vessels play an important role in the regulation of bone turnover and growth.

Another category of tissue regeneration activity that may be attributable to mitoNEET or modulators thereof is tendon/ligament formation. A protein that induces tendon/ligament-like tissue or other tissue formation in circumstances where such tissue is not normally formed has application in the healing of tendon or ligament tears, deformities, and other tendon or ligament defects in humans and other animals. Such a preparation may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of mitoNEET or agonist or antagonist thereto contributes to the repair of congenital, trauma-induced, or other tendon or ligament defects of other origin, and is also useful in cosmetic plastic surgery for attachment or repair of tendons or ligaments. The compositions herein may provide an environment to attract tendon- or ligament-forming cells, stimulate growth of tendon- or ligament-forming cells, induce differentiation of progenitors of tendon- or ligament forming cells, or induce growth of tendon/ligament cells or progenitors ex vivo for return in vivo to effect tissue repair. The compositions herein may also be useful in the treatment of tendinitis, carpal tunnel syndrome, and other tendon or ligament defects. The compositions may also include an appropriate matrix and/or sequestering agent as a carrier as is well known in the art.

MitoNEET or its modulators may also be useful for proliferation of neural cells and for regeneration of nerve and brain tissue, i.e., for the treatment of central and peripheral nervous system disease and neuropathies, as well as mechanical and traumatic disorders, that involve degeneration, death, or trauma to neural cells or nerve tissue. More specifically, mitoNEET or its agonist may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions that may be treated in accordance with the present invention include mechanical and traumatic disorders, such as spinal cord disorders, head trauma, and cerebrovascular diseases such as stroke. Peripheral neuropathies resulting from chemotherapy or other medical therapies may also be treatable using mitoNEET agonist or antagonist thereto.

Ischemia-reperfusion injury is another indication. Endothelial cell dysfunction may be important in both the initiation of, and in regulation of the sequelae of events that occur following ischemia-reperfusion injury.

Rheumatoid arthritis is a further indication. Blood vessel growth and targeting of inflammatory cells through the vasculature is an important component in the pathogenesis of rheumatoid and sero-negative forms of arthritis.

MitoNEET or its modulators thereof may also be administered prophylactically to patients with cardiac hypertrophy, to prevent the progression of the condition, and avoid sudden death, including death of asymptomatic patients. Such preventative therapy is particularly warranted in the case of patients diagnosed with massive left ventricular cardiac hypertrophy (a maximal wall thickness of 35 mm. or more in adults, or a comparable value in children), or in instances when the hemodynamic burden on the heart is particularly strong.

MitoNEET or its modulators may also be useful in the management of atrial fibrillation, which develops in a substantial portion of patients diagnosed with hypertrophic cardiomyopathy. Further indications include angina, myocardial infarctions such as acute myocardial infarctions, and heart failure such as congestive heart failure. Additional non-neoplastic conditions include psoriasis, diabetic and other proliferative retinopathies including retinopathy of prematurity, retrolental fibroplasia, neovascular glaucoma, thyroid hyperplasias (including Grave's disease), corneal and other tissue transplantation, chronic inflammation, lung inflammation, nephrotic syndrome, preeclampsia, ascites, pericardial effusion (such as that associated with pericarditis), and pleural effusion.

In view of the above, mitoNEET or modulators thereof described herein, which are shown to alter or impact endothelial, epithelial, or specialized cell function, proliferation, and/or form, are likely to play an important role in the etiology and pathogenesis of many or all of the disorders noted above, and as such can serve as therapeutic targets to augment or inhibit these processes or for vascular-related drug targeting in these disorders.

Assays for Diabetes

Various assays can be used to test for compounds that interact with mitoNEET and/or mitoNEET associated proteins. For example, in addition to evaluation of direct interaction with mitoNEET, compounds can be evaluated for the ability to affect enzymatic activities that are associated with mitoNEET. This includes, but is not limited to, enzymes involved in fatty acid oxidation particularly in the mitochondria. One example of this approach is to measure the rate of β-oxidation of fatty acyl-CoA esters using isolated membranes or intact mitochondria that contain mitoNEET. Metabolites are measured by the appearance of products as assessed by HPLC or by the rate of reduction of cofactors or substrates (e.g., FIG. 9). Compounds active at modulating mitoNEET activity with respect to these enzymatic activities can then be evaluated in intact cells (e.g., hepatocytes, adipocytes, etc) where intermediates are measured by HPLC following extraction from the cells. Active compounds that modulate mitoNEET activity in these assays and also contain the appropriate properties to become therapeutic agents (e.g., bioavailability, half-live, etc.) would then be expected to produce antidiabetic actions in animal models of diabetes such as lowering circulating glucose and insulin levels and improving insulin-dependent gene expression (e.g., Hofmann, C., Lornez, K., and Colca, J. R. (1991) Endocrinology, 129:1915-1925; Hofmann, C., Lornez, K., and Colca, J. R. (1992) Endocrinology, 130:735-740.)

Assays for Cardiovascular, Endothelial, and Angiogenic Activity

Various assays can be used to test mitoNEET herein for cardiovascular, endothelial, arid angiogenic activity. Such assays include those provided in the Examples below.

Assays for testing for endothelin antagonist activity, as disclosed in U.S. Pat. No. 5,773,414, include a rat heart ventricle binding assay where mitoNEET is tested for its ability to inhibit iodinized endothelin-1 binding in a receptor assay, an endothelin receptor binding assay testing for intact cell binding of radiolabeled endothelin-1 using rabbit renal artery vascular smooth muscle cells, an inositol phosphate accumulation assay where functional activity is determined in Rat-I cells by measuring intra-cellular levels of second messengers, an arachidonic acid release assay that measures the ability of added compounds to reduce endothelin-stimulated arachidonic acid release in cultured vascular smooth muscles, in vitro (isolated vessel) studies using endothelium from male New Zealand rabbits, and in vivo studies using male Sprague-Dawley rats.

Assays for tissue generation activity include, without limitation, those described in WO 95/16035 (bone, cartilage, tendon), WO 95/05846 (nerve, neuronal), and WO 91/07491 (skin, endothelium).

Assays for wound-healing activity include, for example, those described in Winter, Epidermal Wound Healing, Maibach, H I and Rovee, D T, Eds. (Year Book Medical Publishers, Inc., Chicago), pp. 71-112, as modified by the article of Eaglstein and Mertz, J. Invest. Dermatol., 71: 382-384(1978).

There are several cardiac hypertrophy assays. In vitro assays include induction of spreading of adult rat cardiac myocytes. In this assay, ventricular myocytes are isolated from a single (male Sprague-Dawley) rat, essentially following a modification of the procedure described in detail by Piper et al., “Adult ventricular rat heart muscle cells” in Cell Culture Techniques in Heart and Vessel Research, H. M. Piper, ed. (Berlin: Springer-Verlag, 1990), pp. 36-60. This procedure permits the isolation of adult ventricular myocytes and the long-term culture of these cells in the rod-shaped phenotype. Phenylephrine and Prostaglandin F2 (PGF₂) have been shown to induce a spreading response in these adult cells. The inhibition of myocyte spreading induced by PGF₂ or PGF₂ analogs (e.g., fluprostenol) and phenylephrine by various potential inhibitors of cardiac hypertrophy is then tested.

The efficacy for anti-hypertensive action may be measured by indirect or direct means in animal models that demonstrate insulin resistant hypertension (e.g., Hypertension 24(1), 106-10, (1994); Metabolism, Clinical and Experimental 44: 1105-9 (1995)). Efficacy of mitoNEET identified compounds may also be measured directly in vitro (e.g., Journal of Clinical Investigation 96: 354-60, (1995).

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e. from about 8 to about 30 linked nucleo sides). Particularly preferred antisense compounds are antisense oligonucleotides, even more preferably those comprising from about 12 to about 25 nucleobases. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal I linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙ, OCH₃, O(CH₂)ₙNH₂, O(CH₂)ₙCH₃, O(CH₂)ₙONH₂, and O(CH₂ₙON[(CH₂)ₙCH₃)]₂ where n and m are from 1 to about 10. Other preferred oligonucleotides comprise one of the following at the 2′ position: C₁ to C₁₀, (lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ON0₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other preferred modifications include 2′-methoxy (2′-O CH₃), 2′-aminopropoxy (2′-O CH₂ CH₂ CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds, Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,12′, 5,596,091; 5,614,617; 5,750,692, and 5,681,941, each of which is herein incorporated by reference.

Another modification of the oligonucleotides of the invention involves chemically linking to the oligonucleotide one or more moieties or conjugates, which enhance the activity, cellular distribution, or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds, which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease, which cleaves the RNA strand of RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The antisense compounds used in accordance with this invention may be conveniently, and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonuclectides of the invention are prepared as SATE [(S-acetyl-2-thioethyl) phosphate] derivatives according to the methods disclosed in WO 93/24510 to Gosselin et al., published Dec. 9, 1993 or in WO 94/26764 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfoic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits. For therapeutics, an animal, preferably a human, suspected of having a disease or disorder, which can be treated by modulating the expression of mitoNEET, is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The antisense compounds of the invention are useful for research and diagnostics, because these compounds hybridize to nucleic acids encoding mitoNEET, enabling sandwich and other assays to easily be constructed to exploit this fact. Hybridization of the antisense oligonucleotides of the invention with a nucleic acid encoding mitoNEET can be detected by means known in the art. Such means may include conjugation of an enzyme to the oligonucleotide, radiolabelling of the oligonucleotide or any other suitable detection means. Kits using such detection means for detecting the level of mitoNEET in a sample may also be prepared.

The present invention also includes pharmaceutical compositions and formulations, which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer, intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves, and the like may also be useful.

Compositions and formulations for oral administration include powders or granules, suspensions, or solutions in water or non-aqueous media, capsules, sachets, or tablets. Thickeners, flavoring agents, diluents, emulsifiers, dispersing aids, or binders may be desirable.

Compositions and formulations for parenteral, intrathecal or intraventricular administration may include sterile aqueous solutions, which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances, which increase the viscosity of the suspension including, for example, sodium carboxymethylcellulose, sorbitol, and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies, and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention. Emulsions

The compositions of the present invention may be prepared and formulated as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington 's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug, which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosaqe Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic, and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives, and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed phase droplets and by increasing the viscosity of the external phase.

Since emulsions often contain a number of ingredients such as carbohydrates, proteins, sterols, and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallate, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral, and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins, and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil, and amphiphile, which is a single optically isotropic, and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 1852-5). Microemulsions commonly are prepared via a combination of three to five components that include oil, water, surfactant, cosurfactant, and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and triglycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides, or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

There are many organized surfactant structures besides microemulsions that have been studied and used for the formulation of drugs. These include monolayers, micelles, bilayers, and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Noncationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome, which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, P. 245). Important considerations in the preparation of liposome formulations are the lipid surface charge, vesicle size, and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones, and high-molecular weight DNAs have been administered to the skin. The majority of applications resulted in the targeting of the upper epidermis.

Liposomes fall into two broad classes. Cationic liposomes are positively charged liposomes, which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985)

Liposomes, which are pH-sensitive or negatively charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside GM1, galactocerebroside sulfate, and phosphatidylinositol to improve blood half-lives of liposomes. These findings were expounded upon by Gabizon et al. (Proc. Natl. Acad. Sci. U.S.A., 1988, 85, 6949), U.S. Pat. No. 4,837,028 and WO 88/04924, both to Allen et al., disclose liposomes comprising (1) sphingomyelin and (2) the ganglioside Gjor a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C1215G, which contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. Nos. 5,540,935 (Miyazaki et al.) and 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets that are so highly deformable that they are easily able to penetrate through pores that are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285)

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285). Penetration Enhancers

In one embodiment, the present invention employs various penetration enhancers to effect the efficient delivery of nucleic acids particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating nonsurfactants (Lee et al., Critical Reviews in Therapeutic Drug Carrier Systems, 1991, p. 92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-.rac-glycerol), dilaurin, caprylic acid, arachidonic acid, glycerol 1-monocaprate, 1-dodecylazacycloheptan-2-one, acylcarnitines, acylcholines, C1-10 alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds. McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate' and polyoxyethylene-9-lauryl ether (POE) (Lee et al., Critical Reviews in Therapeutic Drug Carrier Systems, 1991, page 92; Swinyard, Chapter 39 In: Remington 's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents:

Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1-33; Buur et al., J Control Rel., 1990, 14, 43-51).

Non-chelating non-surfactants: As used herein, nonchelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers includes, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin, and phenylbutazone (Yamashita et al., J. Pharm. Pharmacol., 1987, 39, 621-626).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′disulfonic acid (Miyao et al., Antisense Res. Dev., 1995, 5, 115-121; Takakura et al., Antisense & Nucl. Acid Drug Dev., 1996, 6, 177-183).

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration, which does not deleteriously react with nucleic acids, can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents, and other suitable additives. Pharmaceutically acceptable organic or inorganic excipients suitable for non-parenteral administration that do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention.’ The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances that increase the viscosity of the suspension including, for example, sodium carboxymethylcellulose, sorbitol, and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). See, generally, The Merck Manual of Diagnosis and Therapy, 15th Ed., Berkow et al., eds., 1987, Rahway, N.J., pages 1206-1228). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively) other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50s found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

While the present invention has been described with specificity in accordance with certain of its preferred embodiments, the following examples serve only to illustrate the invention and are not intended to limit the same.

### EXAMPLES

### Example 1

Nucleoside Phosphoramidites for Oligonucleotide Synthesis Deoxy and 2′-alkoxy Amidites

2′-Deoxy and 2′-methoxy beta-cyanoethyldiisopropyl phosphoramidites are available from commercial sources (e.g. Chemgenes, Needham Mass. or Glen Research, Inc. Sterling Va.). Other 2′-O-alkoxy substituted nucleoside amidites are prepared as described in U.S. Pat. No. 5,506,351, herein incorporated by reference. For oligonucleotides synthesized using 2′-alkoxy amidites, the standard cycle for unmodified oligonucleotides is utilized, except the wait step after pulse delivery of tetrazole and base is increased to 360 seconds.

Oligonucleotides containing 5-methyl-2′-deoxycytidine (5-Me-C) nucleotides are synthesized according to published methods [Sanghvi, et. al., Nucleic Acids Research, 1993, 21, 3197-3203] using commercially available phosphoramidites (Glen Research, Sterling Va. or ChemGenes, Needham Mass.).

2′-Fluoro Amidites

2′-Fluorodeoxyadenosine Amidites

2′-fluoro oligonucleotides are synthesized as described previously [Kawasaki, et. al., J. Med. Chem., 1993, 36, 831-841] and U.S. Pat. No. 5,670,633, herein incorporated by reference. Briefly, the protected nucleoside N6-benzoyl-2′-deoxy-2′-fluoroadenosine is synthesized utilizing commercially available 9-beta-D-arabinofuranosyladenine as starting material and by modifying literature procedures whereby the 2′-alpha-fluoro atom is introduced by a S_{N}2-displacement of a 2′-beta-trityl group. Thus N6-benzoyl-9-beta-D-arabinofuranosyladenine is selectively protected in moderate yield as the 3′,5′-ditetrahydropyranyl (THP) intermediate. Deprotection of the THP and N6-benzoyl groups is accomplished using standard methodologies and standard methods are used to obtain the 5′-dimethoxytrityl-(DMT) and 5′-DMT-3′-phosphoramidite intermediates.

2′-Fluorodeoxyguanosine

The synthesis of 2′-deoxy-2′-fluoroguanosine is accomplished using tetraisopropyldisiloxanyl (TPDS) protected 9-beta-D-arabinofuranosylguanine as starting material, and conversion to the intermediate diisobutyrylarabinofuranosylguanosine. Deprotection of the TPDS group is followed by protection of the hydroxyl group with THP to give diisobutyryl di-THP protected arabinofuranosylguanine. Selective O-deacylation and triflation is followed by treatment of the crude product with fluoride, then deprotection of the THP groups. Standard methodologies are used to obtain the 5′-DMT- and 5′-DMT-3′-phosphoramidites.

2′-Fluorouridine

Synthesis of 2′-deoxy-2′-fluorouridine is accomplished by the modification of a literature procedure in which 2,2′anhydro-1-beta-D-arabinofuranosyluracil is treated with 70% hydrogen fluoride-pyridine. Standard procedures are used to obtain the 5′-DMT and 5′-DMT-3′-phosphoramidites.

2′-Fluorodeoxycytidine

2′-deoxy-2′-fluorocytidine is synthesized via amination of 2′-deoxy-2′-fluorouridine, followed by selective protection to give N4-benzoyl-2′-deoxy-2′-fluorocytidine. Standard procedures are used to obtain the 5′-DMT and 5′-DMT-3′phosphoramidites.

2′-O-(2-Methoxyethyl) Modified Amidites

2′-O-Methoxyethyl-substituted nucleoside amidites are prepared as follows, or alternatively, as per the methods of Martin, P., Helvetica Chimica Acta, 1995, 78, 486-504.

2,2′-Anhydro [1-(beta-D-arabinofuranosyl)-5-methyluridinel

5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) are added to DMF (300 mL). The mixture is heated to reflux, with stirring, allowing the evolved carbon dioxide gas to be released in a controlled manner. After 1 hour, the slightly darkened solution is concentrated under reduced pressure. The resulting syrup is poured into diethylether (2.5 L), with stirring. The product formed a gum. The ether is decanted and the residue is dissolved in a minimum amount of methanol (ca. 400 mL). The solution is poured into fresh ether (2.5 L) to yield a stiff gum. The ether is decanted and the gum is dried in a vacuum oven (60° C. at 1 mm Hg for 24 h) to give a solid that is crushed to a light tan powder. The material is used as is for further reactions (or it can be purified further by column chromatography using a gradient of methanol in ethyl acetate (10-25%) to give a white solid.

2′-O-Methoxyethyl-5-methyluridine

2,2′-Anhydro-5-methyluridine (195 g, 0.81 M), tris(2-methoxyethyl)borate (231 g, 0.98 M) and 2-methoxyethanol (1.2 L) are added to a 2 L stainless steel pressure vessel and placed in a pre-heated oil bath at 160° C. After heating for 48 hours at 155-160° C., the vessel is opened and the solution evaporated to dryness and triturated with MeOH (200 mL). The residue is suspended in hot acetone (1 L). The insoluble salts are filtered, washed with acetone (150 mL) and the filtrate evaporated. The residue (280 g) is dissolved in CH₃CN (600 mL) and evaporated. A silica gel column (3 kg) is packed in CH₂Cl₂/acetone/MeOH (20:5:3) containing 0.5% Et₃NH. The residue is dissolved in CH₂Cl₂ (250 mL) and adsorbed onto silica (150 g) prior to loading onto the column. The product is eluted with the packing solvent to give the title product. Additional material can be obtained by reworking impure fractions.

2′-O-Methoxyethyl-5′-O-dimethoxytrityl-5-methyluridine

2′-O-Methoxyethyl-5-methyluridine (160 g, 0.506 M) is co-evaporated with pyridine (250 mL) and the dried residue dissolved in pyridine (1.3 L). A first aliquot of dimethoxytrityl chloride (94.3 g, 0.278 M) is added and the mixture stirred at room temperature for one hour. A second aliquot of dimethoxytrityl chloride (94.3 g, 0.278 M) is added and the reaction stirred for an additional one hour. Methanol (170 mL) is then added to stop the reaction. The solvent is evaporated and triturated with CH₃CN (200 mL) The residue is dissolved in CHCl (1.5 L) and extracted with 2×500 mL of saturated NaHCO₃ and 2×500 mL of saturated NaCl. The organic phase is dried over Na₂SO₄, filtered, and evaporated. The residue is purified on a 3.5 kg silica gel column, packed and eluted with EtOAc/hexane/acetone (5:5:1) containing 0-5% Et₃NH. The pure fractions are evaporated to give the title product.

3′-O-Acetyl-2′-O-methoxyethyl-5′-O-dimethoxytrityl-5-methyluridine

2′-O-Methoxyethyl-5′-O-dimethoxytrityl-5-methyluridine (106 g, 0.167 M), DMF/pyridine (750 mL of a 3:1 mixture prepared from 562 mL of DMF and 188 mL of pyridine) and acetic anhydride (24.38 mL, 0.258 M) are combined and stirred at room temperature for 24 hours. The reaction is monitored by TLC by first quenching the TLC sample with the addition of MeOH. Upon completion of the reaction, as judged by TLC, MeOH (50 mL) is added and the mixture evaporated at 35° C. The residue is dissolved in CHCl₃ (800 mL) and extracted with 2×200 mL of saturated sodium bicarbonate and 2×200 mL of saturated NaCl. The water layers are back extracted with 200 mL of CHCl₃. The combined organics are dried with sodium sulfate and evaporated to a residue. The residue is purified on a 3.5 kg silica gel column and eluted using EtOAc/hexane(4:1). Pure product fractions are evaporated to yield the title compounds.

3′-O-Acetyl-2′-O-methoxyethyl-5′-O-dimethoxytrityl-5-methyl-4-triazoleuridine

A first solution is prepared by dissolving 3′-O-acetyl-2′-O-methoxyethyl-5′-O-dimethoxytrityl-5-methyluridine (96 g, 0.144 M) in CH₃CN (700 mL) and set aside. Triethylamine (189 mL, 1.44 M) is added to a solution of triazole (90 g, 1.3 M) in CH₃CN (1 L), cooled to −5° C. and stirred for 0.5 h using an overhead stirrer. POCl₃ is added dropwise, over a 30 minute period, to the stirred solution maintained at 0-10° C., and the resulting mixture stirred for an additional 2 hours. The first solution is added dropwise, over a 45 minute period, to the latter solution. The resulting reaction mixture is stored overnight in a cold room. Salts are filtered from the reaction mixture and the solution is evaporated. The residue is dissolved in EtOAc (1 L) and the insoluble solids are removed by filtration. The filtrate is washed with 1×300 mL of NaHCO₃ and 2×300 mL of saturated NaCl, dried over sodium sulfate and evaporated. The residue is triturated with EtOAc to give the title compound.

2′-O-Methoxyethyl-5′-O-dimethoxytrityl-5-methylcytidine

A solution of 3′-O-acetyl-2′-O-methoxyethyl-5′-O-dimethoxytrityl-5-methyl-4-triazoleuridine (103 g, 0.141 M) in dioxane (500 mL) and NH₄OH (30 mL) is stirred at room temperature for 2 hours. The dioxane solution is evaporated and the residue azeotroped with MeOH (2×200 mL). The residue is dissolved in MeOH (300 mL) and transferred to a 2-liter stainless steel pressure vessel. MeOH (400 mL) saturated with NH₃ gas is added and the vessel heated to 100° C. for 2 hours (TLC showed complete conversion). The vessel contents are evaporated to dryness and the residue is dissolved in EtOAc (500 mL) and washed once with saturated NaCl (200 mL). The organics are dried over sodium sulfate and the solvent is evaporated to give the title compound.

N4-Benzoyl-2′-O-methoxyethyl-5′-O-dimethoxytrityl-5-methylcytidine

2′-O-Methoxyethyl-5′-O-dimethoxytrityl-5-methylcytidine (85 g, 0.134 M) is dissolved in DMF (800 mL) and benzoic anhydride (37.2 g, 0.165 M) is added with stirring. After stirring for 3 hours, TLC showed the reaction to be approximately 95% complete. The solvent is evaporated and the residue azeotroped with MeOH (200 mL). The residue is dissolved in CHCl₃ (700 mL) and extracted with saturated NaHCO₃, (2×300 mL) and saturated NaCl (2×300 mL), dried over MgSO₄ and evaporated to give a residue. The residue is chromatographed on a 1.5 kg silica column using EtOAc/hexane (1:1) containing 0-5% Et₃NH as the eluting solvent. The pure product fractions are evaporated to give the title compound.

N4-Benzoyl-2′-O-methoxyethyl-5′-O-dimethoxytrityl-5-methylcytidine-3′-amidite

N4-Benzoyl-2′-O-methoxyethyl-5′-O-dimethoxytrityl-5-methylcytidine (74 g, 0.10 M) is dissolved in CH₂Cl₂ (1 L) Tetrazole diisopropylamine (7.1 g) and 2-cyanoethoxy-tetra(isopropyl)phosphite (40.5 mL, 0.123 M) are added with stirring, under a nitrogen atmosphere. The resulting mixture is stirred for 20 hours at room temperature (TLC showed the reaction to be 95% complete). The reaction mixture is extracted with saturated NaHCO₃ (1×300 mL) and saturated NaCl (3×300 mL). The aqueous washes are back-extracted with CH₂Cl₂ (300 mL), and the extracts are combined, dried over MgSO₄, and concentrated. The residue obtained is chromatographed on a 1.5 kg silica column using EtOAc/hexane (3:1) as the eluting solvent. The pure fractions were combined to give the title compound.

2′-O-(Aminooxyethyl)nucleoside amidites and 2′-O-(dimethylaminooxyethyl) Nucleoside Amidites

2′-(Dimethylaminooxyethoxy) Nucleoside Amidites

2′-(Dimethylaminooxyethoxy) nucleoside amidites [also known in the art as 2′-O-(dimethylaminooxyethyl) nucleoside amidites] are prepared as described in the following paragraphs. Adenosine, cytidine and guanosine nucleoside amidites are prepared similarly to the thymidine (5-methyluridine) except the exocyclic amines are protected with a benzoyl moiety in the case of adenosine and cytidine and with isobutyryl in the case of guanosine.

5′-O-tert-Butyldiphenylsilyl-O²-2′-anhydro-5-methyluridine

O²-2′-anhydro-5-methyluridine (Pro. Bio. Sint., Varese, Italy, 100.0 g, 0.4′6 mmol), dimethylaminopyridine (0.66 g, 0.013 eq, 0.0054 mmol) are dissolved in dry pyridine (500 ml) at ambient temperature under an argon atmosphere and with mechanical stirring tert-Butyldiphenylchlorosilane (125.8 g, 119.0 mL, 1.1 eq, 0.458 mmol) is added in one portion. The reaction is stirred for 16 h at ambient temperature. TLC (Rf 0.22, ethyl acetate) indicated a complete reaction. The solution is concentrated under reduced pressure to a thick oil. This is partitioned between dichloromethane (1 L) and saturated sodium bicarbonate (2×1 L) and brine (1 L). The organic layer is dried over sodium sulfate and concentrated under reduced pressure to a thick oil. The oil is dissolved in a 1:1 mixture of ethyl acetate and ethyl ether (600 mL) and the solution is cooled to −10° C. The resulting crystalline product is collected by filtration, washed with ethyl ether (3×200 mL), and dried (40° C., 1 mm Hg, 24 h) to a white solid

5′-O-tert-Butyldiphenylsilyl-2′-O-(2-hydroxyethyl)-5-methyluridine

In a 2 L stainless steel, unstirred pressure reactor is added borane in tetrahydrofuran (1.0 M, 2.0 eq, 622 mL). In the fume hood and with manual stirring, ethylene glycol (350 mL, excess) is added cautiously at first until the evolution of hydrogen gas subsides. 5′-O-tert-Butyldiphenylsilyl-O²-2′anhydro-5-methyluridine (149 g, 0.3′1 mol) and sodium bicarbonate (0.074 g, 0.003 eq) are added with manual stirring. The reactor is sealed and heated in an oil bath until an internal temperature of 160° C. is reached and then maintained for 16 h (pressure <100 psig). The reaction vessel is cooled to ambient and opened. TLC (Rf 0.67 for desired product and Rf 0.82 for ara-T side product, ethyl acetate) indicated about 70% conversion to the product. In order to avoid additional side product formation, the reaction is stopped, concentrated under reduced pressure (10 to 1 mm, Hg) in a warm water bath (40-100° C.) with the more extreme conditions used to remove the ethylene glycol. [Alternatively, once the low boiling solvent is gone, the remaining solution can be partitioned between ethyl acetate and water. The product will be in the organic phase.] The residue is purified by column chromatography (2 kg silica gel, ethyl acetate-hexanes gradient 1:1 to 4:1). The appropriate fractions are combined, stripped, and dried to product as a white crisp foam, contaminated starting material, and pure reusable starting material.

2′-O-([2-phthalimidoxy)ethyl]-5′-t-butyldiphenylsilyl-5-methyluridine

5′-O-tert-Butyldiphenylsilyl-2′-O-(2-hydroxyethyl)-5-methyluridine (20 g, 36.98 mmol) is mixed with triphenylphosphine (11.63 g, 44.36 mmol) and N-hydroxyphthalimide (7.24 g, 44.36 mmol). It is then dried over P₂O₅ under high vacuum for two days at 40° C. The reaction mixture is flushed with argon and dry THF (369.8 mL, Aldrich, sure seal bottle) is added to get a clear solution. Diethyl-azodicarboxylate (6.98 mL, 44.36 mmol) is added dropwise to the reaction mixture. The rate of addition is maintained such that resulting deep red coloration is just discharged before adding the next drop. After the addition is complete, the reaction is stirred for 4 hrs. By that time TLC showed the completion of the reaction (ethylacetate:hexane, 60:40). The solvent is evaporated in vacuum. Residue obtained is placed on a flash column and eluted with ethyl acetate:hexane (60:40), to get 2′-O-([2-phthalimidoxy)ethyl]-5′-t-butyldiphenylsilyl-5-methyluridine as white foam.

5′-O-tert-butyldiphenylsilyl-2′-O-[(2-formadoximinooxy)ethyl]-5-methyluridine

2′-O-([2-phthalimidoxy)ethyl]-5′-t-butyldiphenylsilyl-5-methyluridine (3.1 g, 4.5 mmol) is dissolved in dry CH₂Cl₂ (4.5 mL) and methylhydrazine (300 mL, 4.64 mmol) is added dropwise at −10° C. to 0° C. After 1 h the mixture is filtered, the filtrate is washed with ice cold CH₂Cl₂ and the combined organic phase is washed with water, brine and dried over anhydrous Na₂SO₄. The solution is concentrated to get 2′-O(aminooxyethyl) thymidine, which is then dissolved in MeOH (67.5 mL). To this formaldehyde (20% aqueous solution, w/w, 1.1 eq.) is added and the resulting mixture is stirred for 1 h. Solvent is removed under vacuum; residue chromatographed to get 5′-O-tert-butyldiphenylsilyl-2′-O-[(2-formadoximinooxy) ethyl]-5-methyluridine as white foam. 5′-O-tert-Butyldiphenylsilyl-2′-O-[N,N-dimethylaminooxyethyl]-5-methyluridine

5′-O-tert-butyldiphenylsilyl-2′-O-[(2-formadoximinooxy)ethyl]-5-methyluridine (1.77 g, 3.12 mmol) is dissolved in a solution of 1M pyridinium p-toluenesulfonate (PPTS) in dry MeOH (30.6 mL). Sodium cyanoborohydride (0.39 g, 6.13 mmol) is added to this solution at 110° C. under inert atmosphere. The reaction mixture is stirred for 10 minutes at 110° C. After that the reaction vessel is removed from the ice bath and stirred at room temperature for 2 h, the reaction monitored by TLC (5% MeOH in CH₂Cl₂). Aqueous NaHCO₃ solution (5%, 10 mL) is added and extracted with ethyl acetate (2×20 mL). Ethyl acetate phase is dried over anhydrous Na₂SO₄, evaporated to dryness. Residue is dissolved in a solution of 1M PPTS in MeOH (30.6 mL). Formaldehyde (20% w/w, 30 mL, 3.37 mmol) is added and the reaction mixture is stirred at room temperature for 10 minutes. Reaction mixture cooled to 10° C. in an ice bath, sodium cyanoborohydride (0.39 g, 6.13 mmol) is added, and reaction mixture stirred at 10° C. for 10 minutes. After 10 minutes, the reaction mixture is removed from the ice bath and stirred at room temperature for 2 hrs. To the reaction mixture 5% NaHCO₃ (25 mL) solution is added and extracted with ethyl acetate (2×25 mL). Ethyl acetate layer is dried over anhydrous Na₂SO₄ and evaporated to dryness. The residue obtained is purified by flash column chromatography and eluted with 5% MeOH in CH₂Cl₂ to get 5′-O-tertbutyldiphenylsilyl-2′-O-[N,N-dimethylaminooxyethyl]-5-methyluridine as a white foam.

2′-O-(dimethylaminooxyethyl)-5-methyluridine

Triethylamine trihydrofluoride (3.91 mL, 24.0 mmol) is dissolved in dry THF and triethylamine (1.67 mL, 12 mmol, dry, kept over KOH). This mixture of triethylamine-2 HF is then added to 5′-O-tert-butyldiphenylsilyl-2′-O-[N,N-dimethylaminooxyethyl]-5-methyluridine (1.40 g, 2.4 mmol) and stirred at room temperature for 24 hrs. Reaction is monitored by TLC (5% MeOH in CH₂Cl₂). Solvent is removed under vacuum and the residue placed on a flash column and eluted with 10% MeOH in CH₂Cl₂ to get 2′-O-(dimethylaminooxyethyl)-5-methyluridine.

5′-O-DMT-2′-O-(dimethylaminooxyethyl)-5-methyluridine

2′-O-(dimethylaminooxyethyl)-5-methyluridine (750 mg, 2.17 mmol) is dried over P₂O₅ under high vacuum overnight at 40° C. It is then co-evaporated with anhydrous pyridine (20 mL). The residue obtained is dissolved in pyridine (11 mL) under argon atmosphere. 4-dimethylaminopyridine (26.5 mg, 2.60 mmol), 4,4′-dimethoxytrityl chloride (880 mg, 2.60 mmol) is added to the mixture and the reaction mixture is stirred at room temperature until all of the starting material disappeared. Pyridine is removed under vacuum and the residue chromatographed and eluted with 10% MeOH in CH₂Cl₂ (containing a few drops of pyridine) to get 5′-O-DMT-2′-0(dimethylamino-oxyethyl)-5-methyluridine.

5′-O-DMT-2′-O-(2-N,N-dimethylaminooxyethyl)-5-methyluridine-3′-[(2-cyanoethyl)-N,N-diisopropylphosphoramidite]

5′-O-DMT-2′-O-(dimethylaminooxyethyl)-5-methyluridine (1.08 g, 1.67 mmol) is co-evaporated with toluene (20 mL). To the residue N,N-diisopropylamine tetrazonide (0.29 g, 1.67 mmol) is added and dried over P20, under high vacuum overnight at 40° C. Then the reaction mixture is dissolved in anhydrous acetonitrile (8.4 mL) and 2-cyanoethyl-N,N,N¹,N¹-tetraisopropylphosphoramidite (2.12 mL, 6.08 mmol) is added. The reaction mixture is stirred at ambient temperature for 4 hrs under inert atmosphere. The progress of the reaction is monitored by TLC (hexane:ethyl acetate 1:1). The solvent is evaporated, then the residue is dissolved in ethyl acetate (70 mL) and washed with 5% aqueous NaHCO₃ (40 mL). Ethyl acetate layer is dried over anhydrous Na₂SO₄ and concentrated. Residue obtained is chromatographed (ethyl acetate as eluent) to get 5′-O-DMT-2′-O-(2-N,N-dimethylaminooxyethyl)-5-methyluridine-3′-[(2-cyanoethyl)-N,N-diisopropylphosphoramidite] as a foam.

2′-(Aminooxyethoxy) Nucleoside Amidites

2′-(Aminooxyethoxy) nucleoside amidites [also known in the art as 2′-O-(Aminooxyethyl)nucleoside amidites] are prepared as described in the following paragraphs. Adenosine, cytidine and thymidine nucleoside amidites are prepared similarly.

N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite]

The 2′-O-aminooxyethyl guanosine analog may be obtained by selective 2′-O-alkylation of diaminopurine riboside. Multigram quantities of diaminopurine riboside may be purchased from Schering AG (Berlin) to provide 2′-O-(2-ethylacetyl) diaminopurine riboside along with a minor amount of the 3′-O-isomer. 2′-O-(2-ethylacetyl) diaminopurine riboside may be resolved and converted to 2′-O-(2ethylacetyl)guanosine by treatment with adenosine deaminase. (McGee, D. P. C., Cook, P. D., Guinosso, C. J., WO 94/02501 A1 940203.) Standard protection procedures should afford 2′-O-(2-ethylacetyl)-5′-O-(4,4′-dimethoxytrityl)guanosine and 2-N-isobutyryl-6-O-diphenylcarbamoyl-2′-O-(2-ethylacetyl)-5′-O-(4,4′-dimethoxytrityl)guanosine which may be reduced to provide 2-N-isobutyryl-6-O-diphenylcarbamoyl-2′-O-(2-ethylacetyl)-5′-O-(4,4′-dimethoxytrityl)guanosine. As before the hydroxyl group may be displaced by N-hydroxyphthalimide via a Mitsunobu reaction, and the protected nucleoside may phosphitylated as usual to yield 2-N-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramiditel.

2′-dimethylaminoethoxyethoxy (2′-DMAEOE) Nucleoside Amidites

2′-dimethylaminoethoxyethoxy nucleoside amidites (also known in the art as 2′-O-dimethylaminoethoxyethyl, i.e., 2′0-CH₂—O—CH₂—N(CH₂)₂, or 2′-DMAEOE nucleoside amidites) are prepared as follows. Other nucleoside amidites are prepared similarly.

2′-O-[2(2-N,N-dimethylaminoethoxy)ethyl]-5-methyl Uridine

2[2-(Dimethylamino)ethoxy]ethanol (Aldrich, 6.66 g, 50 mmol) is slowly added to a solution of borane in tetrahydrofuran (1 M, 10 mL, 10 mmol) with stirring in a 100 mL bomb. Hydrogen gas evolves as the solid dissolves. O²-, 2′-anhydro-5-methyluridine (1.2 g, 5 mmol), and sodium bicarbonate (2.5 mg) are added and the bomb is sealed, placed in an oil bath, and heated to 155° C. for 26 hours. The bomb is cooled to room temperature and opened. The crude solution is concentrated and the residue partitioned between water (200 mL) and hexanes (200 mL). The excess phenol is extracted into the hexane layer. The aqueous layer is extracted with ethyl acetate (3×200 mL) and the combined organic layers are washed once with water, dried over anhydrous sodium sulfate, and concentrated. The residue is columned on silica gel using methanol/methylene chloride 1:20 (which has 2% triethylamine) as the eluent. As the column fractions are concentrated a colorless solid forms which is collected to give the title compound as a white solid.

5′-O-dimethoxytrityl-2′-O-[2(2-N,N-dimethylaminoethoxy) ethyl)]-5-methyl Uridine

To 0.5 g (1.3 mmol) of 2′-O-[2(2-N,N-dimethylaminoethoxy)ethyl)1-5-methyl uridine in anhydrous pyridine (8 mL), triethylamine (0.36 mL) and dimethoxytrityl chloride (DMT-Cl, 0.87 g, 2 eq.) are added and stirred for 1 hour. The reaction mixture is poured into water (200 mL) and extracted with CH₂Cl₂ (2×200 mL). The combined CH₂Cl₂ layers are washed with saturated NaHCO₃ solution, followed by saturated NaCl solution, and dried over anhydrous sodium sulfate. Evaporation of the solvent followed by silica gel chromatography using MeOH:CH₂Cl₂:Et₃N (20:1, v/v, with 1% triethylamine) gives the title compound.

5′-O-Dimethoxytrityl-2′-O-[2(2-N,N-dimethylaminoethoxy)ethyl)]-5-methyl uridine-3′-O-(cyanoethyl-N,N-diisopropyl)phosphoramidite

Diisopropylaminotetrazolide (0.6 g) and 2-cyanoethoxyN,N-diisopropyl phosphoramidite (1.1 mL, 2 eq.) are added to a solution of 5′-O-dimethoxytrityl-2′-O-[2(2-N,N-dimethylaminoethoxy)ethyl)]-5-methyluridine (2.17 g, 3 mmol) dissolved in CH₂Cl₂ (20 mL) under an atmosphere of argon. The reaction mixture is stirred overnight and the solvent evaporated. The resulting residue is purified by silica gel flash column chromatography with ethyl acetate as the eluent to give the title compound.

### Example 2

Oligonucleotide Synthesis

Unsubstituted and substituted phosphodiester (P═O) oligonucleotides are synthesized on an automated DNA synthesizer (Applied Biosystems model 380B) using standard phosphoramidite chemistry with oxidation by iodine.

Phosphorothioates (P═S) are synthesized as for the phosphodiester oligonucleotides except the standard oxidation bottle is replaced by 0.2 M solution of 3H-1,2-benzodithiole-3-one 1,1-dioxide in acetonitrile for the stepwise thiation of the phosphite linkages. The thiation wait step is increased to 68 sec and is followed by the capping step. After cleavage from the CPG column and deblocking in concentrated ammonium hydroxide at 55° C. (18 h), the oligonucleotides are purified by precipitating twice with 2.5 volumes of ethanol from a 0.5 M NaCl solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. Nos. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in WO 94/17093 and WO 94/02499 herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

### Example 3

Oligonucleoside Synthesis

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825; 5,386,023; 5,489,677; 5,602,240; and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 4

PNA Synthesis

Peptide nucleic acids (PNAs) are prepared in accordance with any of the various procedures referred to in Peptide Nucleic Acids (PNA): Synthesis, Properties and Potential Applications, Bioorganic & Medicinal Chemistry, 1996, 4, 523. They may also be prepared in accordance with U.S. Pat. Nos. 5,539,082; 5,700,922; and 5,719,262, herein incorporated by reference.

### Example 5

Synthesis of Chimeric Oligonucleotides

Chimeric oligonucleotides, oligonucleosides, or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

[2′-O-Me]—[2′-deoxy]—[2′-O-Me] Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 380B, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by increasing the wait step after the delivery of tetrazole and base to 600 s repeated four times for RNA and twice for 2′-O-methyl. The fully protected oligonucleotide is cleaved from the support and the phosphate group is deprotected in 3:1 ammonia/ethanol at room temperature overnight then lyophilized to dryness. Treatment in methanolic ammonia for 24 hrs at room temperature is then done to deprotect all bases and sample is again lyophilized to dryness. The pellet is resuspended in 1M TBAF in THF for 24 hrs at room temperature to deprotect the 2′ positions. The reaction is then quenched with 1 M TEAA and the sample is then reduced to ½ volume by rotovac before being desalted on a G25 size exclusion column. The oligo recovered is then analyzed spectrophotometrically for yield and for purity by capillary electrophoresis and by mass spectrometry.

[2′-O-(2-Methoxyethyl)]—12′-deoxy]—[2′-O-(Methoxyethyl)] Chimeric Phosphorothioate Oligonucleotides

[2′-O-(2-methoxyethyl)]—[2′-deoxy]-[-2′-O-(methoxyethyl)] chimeric phosphorothioate oligonucleotides are prepared as per the procedure above for the 2′-O-methyl chimeric oligonucleotide, with the substitution of phorothioate oligonucleotides are prepared as per the procedure abo 2′-O-(methoxyethyl) amidites for the 2′-O-methyl amidites.

[2′-O-(2-Methoxyethyl)Phosphodiester]—[2′-deoxy Phosphorothioate]—[2′-O-(2-Methoxyethyl)] Phosphodiester] Chimeric Oligonucleotides

[2′-O-(2-methoxyethyl phosphodiester]—[2′-deoxy phosphorothioate]—[2′-O-(methcixyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidization with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides, and mixed chimeric oligonucleotides/oligonucleosides are synthesized according to U.S. Pat. No. 5,623,065, herein incorporated by reference.

### Example 6

Oligonucleotide Isolation

After cleavage from the controlled pore glass column (Applied Biosystems) and deblocking in concentrated ammonium hydroxide at 55° C. for 18 hours, the oligonucleotides or oligonucleosides are purified by precipitation twice out of 0.5 M NaCl with 2.5 volumes ethanol. Synthesized oligonucleotides are analyzed by polyacrylamide gel electrophoresis on denaturing gels and judged to be at least 85% full-length material. The relative amounts of phosphorothioate and phosphodiester linkages obtained in synthesis are periodically checked by “P nuclear magnetic resonance spectroscopy, and for some studies oligonucleotides are purified by HPLC, as described by Chiang et al., J. Biol. Chem. 1991, 266, 18162-18171.

### Example 7

Oligonucleotide Synthesis—96 Well Plate Format

Oligonucleotides are synthesized via solid phase P(III) phosphoramidite chemistry on an automated synthesizer capable of assembling 96 sequences simultaneously in a standard 96 well format. Phosphodiester internucleotide linkages are afforded by oxidation with aqueous iodine. Phosphorothioate internucleotide linkages are generated by sulfurization utilizing 3,H-1,2 benzodithiole-3-one 1,1 dioxide (Beaucage Reagent) in anhydrous acetonitrile. Standard base-protected beta-cyanoethyldiisopropyl phosphoramidites can be purchased from commercial vendors (e.g. PE-Applied Biosystems, Foster City, Calif., or Pharmacia, Piscataway, N.J.). Non-standard nucleosides are synthesized as per known literature or patented methods. They are utilized as base protected betacyanoethyldiisopropyl phosphoramidites.

Oligonucleotides are cleaved from support and deprotected with concentrated NH₄OH at elevated temperature (55-60° C.) for 12-16 hours and the released product then dried in vacuo. The dried product is then re-suspended in sterile water to afford a master plate from which all analytical and test plate samples are then diluted utilizing robotic pipettors.

### Example 8

Oligonucleotide Analysis—96 Well Plate Format

The concentration of oligonucleotide in each well is assessed by dilution of samples and UV absorption spectroscopy. The full-length integrity of the individual products is evaluated by capillary electrophoresis (CE) in either the 96 well format (Beckman P/ACE™ MDQ) or, for individually prepared samples, on a commercial CE apparatus (e.g., Beckman P/ACE™ 5000, ABI 270). Base and backbone composition is confirmed by mass analysis of the compounds utilizing electrospray-mass spectroscopy. All assay test plates are diluted from the master plate using single and multi-channel robotic pipettors. Plates are judged to be acceptable if at least 85% of the compounds on the plate are at least 85% full length.

### Example 9

Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following 6 cell types are provided for illustrative purposes, but other cell types can be routinely used, provided that the target is expressed in the cell type chosen. This can be readily determined by methods routine in the art, for example Northern blot analysis, Ribonuclease protection assays, or RT-PCR.

T-24 Cells:

The human transitional cell bladder carcinoma cell line T-24 is obtained from the American Type Culture Collection (ATCC) (Manassas, Va.). T-24 cells are routinely cultured in complete McCoy's 5A basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells are routinely passaged by trypsinization and dilution when they reached 90% confluence. Cells are seeded into 96-well plates (Falcon-Primaria #3872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 can be obtained from the American Type Culture Collection (ATCC) (Manassas, Va.). A549 cells are routinely cultured in DMEM basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells are routinely passaged by trypsinization and dilution when they reached 90% confluence.

NHDF Cells:

Human neonatal dermal fibroblast (NHDF) can be obtained from the Clonetics Corporation (Walkersville Md.). NHDFs are routinely maintained in Fibroblast Growth Medium (Clonetics Corporation, Walkersville Md.) supplemented as recommended by the supplier. Cells are maintained for up to 10 passages as recommended by the supplier.

HEK Cells:

Human embryonic keratinocytes (HEK) can be obtained from the Clonetics Corporation (Walkersville Md.). HEKs are routinely maintained in Keratinocyte Growth Medium (Clonetics Corporation, Walkersville Md.) formulated as recommended by the supplier. Cells are routinely maintained for up to 10 passages as recommended by the supplier.

MCF-7 Cells:

The human breast carcinoma cell line MCF-7 is obtained from the American Type Culture Collection (Manassas, Va.). MCF-7 cells are routinely cultured in DMEM low glucose (Gibco/Life Technologies, Gaithersburg, Md.) supplemented with 10% fetal calf serum (Gibco/Life Technologies, Gaithersburg, Md.). Cells are routinely passaged by trypsinization and dilution when they reached 90% confluence. Cells are seeded into 96-well plates (Falcon-Primaria #3872) at a density of 7000 cells/well for use in RT-PCR analysis.

For Northern blotting or other analyses, cells may be seeded onto 100 mm or other standard tissue culture plates and treated similarly, using appropriate volumes of medium and oligonucleotide.

LA4 Cells:

The mouse lung epithelial cell line LA4 is obtained from the American Type Culture Collection (Manassas, Va.). LA4 cells are routinely cultured in F12K medium (Gibco/Life Technologies, Gaithersburg, Md.) supplemented with 15% fetal calf serum (Gibco/Life Technologies, Gaithersburg, Md.). Cells are routinely passaged by trypsinization and dilution when they reached 90% confluence. Cells are seeded into 96-well plates (Falcon-Primaria #3872) at a density of 3000-6000 cells/well for use in RT-PCR analysis.

For Northern blotting or other analyses, cells may be seeded onto 100 mm or other standard tissue culture plates and treated similarly, using appropriate volumes of medium and oligonucleotide.

Treatment with Antisense Compounds:

When cells reached 80% confluence, they are treated with oligonucleotide. For cells grown in 96-well plates, wells are washed once with 200 μL OPTI-MEM™-1 reduced-serum medium (Gibco BRL) and then treated with 130 μL of OPTI-MEMTM™-1 containing 3.75 μg/mL LIPOFECTIN™ (Gibco BRL) and the desired concentration of oligonucleotide. After 4-7 hours of treatment, the medium is replaced with fresh medium. Cells are harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations.

### Example 10

Analysis of Oligonucleotide Inhibition of mitoNEET Expression

Antisense modulation of mitoNEET expression can be assayed in a variety of ways known in the art. For example, mitoNEET mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions. Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed as multiplexable. Other methods of PCR are also known in the art.

Protein levels of mitoNEET can be quantitated in a variety of ways well known in the art, such as immunoprecipitation, Western blot analysis (immunoblotting), ELISA or fluorescence-activated cell sorting (FACS). Antibodies directed to mitoNEET can be identified and obtained from a variety of sources, such as the MSRS catalog of antibodies (Aerie Corporation, Birmingham, MI), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley Sons, Inc., 1997.

Immunoprecipitation methods are standard in the art and can be found at, for example, Ausubel, F. M. et al., Current Protocols in Molecular Biology, Volume 2, pp. 10.16.110.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Example 11

Poly(A)+ mRNA Isolation

Poly(A)+ mRNA is isolated according to Miura et al., Clin. Chem., 1996, 42, 1758-1764. Other methods for poly(A)+ mRNA isolation are taught in, for example, Ausubel, F. M. et al., Current Protocols in Molecular Biology, Volume 1, pp. 4.5.1-4.5.3, John Wiley & Sons, Inc., 1993. Briefly, for cells grown on 96-well plates, growth medium is removed from the cells and each well is washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) is added to each well, the plate is gently agitated and then incubated at room temperature for five minutes. 55 μL of lysate is transferred to Oligo d(T) coated 96-well plates (AGCT Inc., Irvine Calif.). Plates are incubated for 60 minutes at room temperature, washed 3 times with 200 μL of wash buffer (10 mM Tris-HC 1 pH 7.6, 1 mM EDTA, 0.3 M NaCl). After the final wash, the plate is blotted on paper towels to remove excess wash buffer and then air-dried for 5 minutes. 60 pL of elution buffer (5 mM Tris-HCl pH 7.6), preheated to 70° C. is added to each well, the plate is incubated on a 90° C. hot plate for 5 minutes, and the eluate is then transferred to a fresh 96-well plate.

Cells grown on 100 mm or other standard plates may be treated similarly, using appropriate volumes of all solutions.

### Example 12

Total RNA Isolation

Total mRNA is isolated using an RNEASY 96™ kit and buffers purchased from Qiagen Inc. (Valencia Calif.) following the manufacturer's recommended procedures. Briefly, for cells grown on 96-well plates, growth medium is removed from the cells and each well is washed with 200 μL cold PBS. 100 μL Buffer RLT is added to each well and the plate vigorously agitated for 20 seconds. 100 μL of 70% ethanol is then added to each well and the contents mixed by pipetting three times up and down. The samples are then transferred to the RNEASY 96™ well plate attached to a QIAVAC™ manifold fitted with a waste collection tray and attached to a vacuum source. Vacuum is applied for 15 seconds. 1 mL of Buffer RW1 is added to each well of the RNEASY 96™ plate and the vacuum again applied for 15 seconds. 1 mL of Buffer RPE is then added to each well of the RNEASY 96™ plate and the vacuum applied for a period of 15 seconds. The Buffer RPE wash is then repeated and the vacuum is applied for an additional 10 minutes. The plate is then removed from the QIAVAC™ manifold and blotted dry on paper towels. The plate is then re-attached to the QIAVAC™ manifold fitted with a collection tube rack containing 1.2 mL collection tubes. RNA is then eluted by pipetting 60 μL water into each well, incubating one minute, and then applying the vacuum for 30 seconds. The elution step is repeated with an additional 60 μL water.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 13

Real-time Quantitative PCR Analysis of mitoNEET mRNA Levels

Quantitation of mitoNEET mRNA levels is determined by real-time quantitative PCR using the ABI PRISM™ 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE, FAM™, or VIC, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ 7700 Sequence Detection System. In each assay, a series of parallel reactions containing serial dilutions of mRNA from untreated control samples generates a standard curve that is used to quantitate the percent inhibition after antisense oligonucleotide treatment of test samples.

PCR reagents can be obtained from PE-Applied Biosystems, Foster City, Calif. RT-PCR reactions are carried out by adding 25 μL PCR cocktail (1×TAQMAN™ buffer A, 5.5 MM MgCl₂, 300 μM each of dATP, dCTP and dGTP, 600 μM of dUTP, 100 nM each of forward primer, reverse primer, and probe, 20 Units RNAse inhibitor, 1.25 Units AMPLITAQ GOLD™, and 12.5 Units MuLV reverse transcriptase) to 96 well plates containing 25 μL poly(A) mRNA solution. The RT reaction is carried out by incubation for 30 minutes at 48° C. Following a 10 minute incubation at 95° C. to activate the AMPLITAQ GOLD™, 40 cycles of a two-step PCR protocol are carried out: 95° C. for 15 seconds (denaturation) followed by 60° C. for 1.5 minutes (annealing/extension).

Probes and primers to human mitoNEET were designed to hybridize to a human mitoNEET sequence, using published sequence, information (GenBank accession number NM_{—}018464, incorporated herein as FIG. 1). For human mitoNEET the PCR primers were:

forward primer: TCCTAGTGCACACGCCTTTG SEQ ID NO:618

reverse primer: ACTCGTACGCTGGAACTGGAA SEQ ID NO: 619 and the PCR

probe is: FAM™-AAGCGACGGCGCCATGAGTCTG SEQ ID NO:620-TAMRA

where FAM™ (PE-Applied Biosystems, Foster City, Calif.) is the fluorescent reporter dye) and TAMRA (PE-Applied Biosystems, Foster City, Calif.) is the quencher dye. For human cyclophilin the PCR primers were:

forward primer: CCCACCGTGTTCTTCGACAT SEQ ID NO:621

reverse primer: TTTCTGCTGTCTTTGGGACCTT SEQ ID NO; 622 and the PCR

probe is:5′JOE-CGCGTCTCCTTTGAGCTGTTTGCA SQE ID NO;623-TAMRA 3′ where JOE (PE-Applied Biosystems, Foster City, Calif.) is the fluorescent reporter dye) and TAMRA (PE-Applied Biosystems, Foster City, Calif.) is the quencher dye.

### Example 14

Antisense Inhibition of Human mitoNEET Expression by Chimeric Phosphorothioate Oligonucleotides Having 2′-MOE Wings and a Deoxy Gap

In accordance with the present invention, a series of oligonucleotides are designed to target different regions of the human mitoNEET RNA, using published sequences (GenBank accession number NM_{—}018464, incorporated herein as SEQ ID NO: 2 in FIG. 1). The oligonucleotides are shown in Table 1. “Position” indicates the first (5′-most) nucleotide number on the particular target sequence to which the oligonucleotide binds. The indicated parameters for each oligo were predicted using RNAstructure 3.7 by David H. Mathews, Michael Zuker, and Douglas H. Turner. The parameters are described either as free energy (The energy that is released when a reaction occurs. The more negative the number, the more likely the reaction will occur. All free energy units are in kcal/mol.) or melting temperature (the temperature at which two anneal strands of polynucleic acid separate. The higher the temperature, greater the affinity between the 2 strands.) When designing an antisense oligonucleotide (oligomers) that will bind with high affinity, it is desirable to consider the structure of the target RNA strand and the antisense oligomer. Specifically, for an oligomer to bind tightly (in the table described as ‘duplex formation’), it should be complementary to a stretch of target RNA that has little self-structure (in the table the free energy of which is described as ‘target structure’). Also, the oligomer should have little self-structure, either intramolecular (in the table the free energy of which is described as ‘intramolecular oligo’) or bimolecular (in the table the free energy of which is described as ‘intermolecular oligo’). Breaking up any self-structure amounts to a binding penalty. All compounds in Table 1 are chimeric oligonucleotides (“gapmers”) 20 nucleotides in length, composed of a central “gap” region consisting of ten 2′deoxynucleotides, which is flanked on both sides (5′ and 3′ directions) by four-nucleotide “wings”. The wings are composed of 2′-methoxyethyl (2′-MOE) nucleotides. The internucleoside (backbone) linkages are phosphorothioate (P═S) throughout the oligonucleotide. Cytidine residues in the 2′-MOE wings are 5-methylcytidines. All cytidine residues are 5-methylcytidines.

TABLE 1

| | | | | | | | |
|---|---|---|---|---|---|---|---|
| | | kcal/mol | kcal/mol | deg C. | kcal/mol | Intra- | Inter- |
| | | total | duplex | Tm of | target | molecular | molecular |
| position | oligo | binding | formation | Duplex | structure | oligo | oligo |

| | | | | | | | |
|---|---|---|---|---|---|---|---|
| 382 | TTGTCTCCAGTCTCTTCGTT | −24.4 | −26.1 | 78.4 | −1.7 | 0 | −3 |
| | SEQ.ID.NO:1 | | | | | | |
| 380 | GTCTCCAGTCTCTTCGTTAT | −24 | −25.7 | 77.5 | −1.7 | 0 | −3 |
| | SEQ.ID.NO:2 | | | | | | |
| 381 | TGTCTCCAGTCTCTTCGTTA | −24 | −25.7 | 77.3 | −1.7 | 0 | −3 |
| | SEQ.ID.NO:3 | | | | | | |
| 383 | ATTGTCTCCAGTCTCTTCGT | −23.8 | −26 | 77.9 | −2.2 | 0 | −3 |
| | SEQ.ID.NO:4 | | | | | | |
| 378 | CTCCAGTCTCTTCGTTATGT | −23.6 | −25.3 | 75.4 | −1.7 | 0 | −3 |
| | SEQ.ID.NO:5 | | | | | | |
| 377 | TCCAGTCTCTTCGTTATGTT | −22.8 | −24.5 | 73.7 | −1.7 | 0 | −3 |
| | SEQ.ID.NO:6 | | | | | | |
| 379 | TCTCCAGTCTCTTCGTTATG | −22.8 | −24.5 | 73.5 | −1.7 | 0 | −3 |
| | SEQ.ID.NO:7 | | | | | | |
| 376 | CCAGTCTCTTCGTTATGTTT | −22.5 | −24.2 | 72.3 | −1.7 | 0 | −3 |
| | SEQ.ID.NO:8 | | | | | | |
| 373 | GTCTCTTCGTTATGTTTTGT | −21.2 | −22.8 | 70.7 | −1.5 | 0 | −3 |
| | SEQ.ID.NO:9 | | | | | | |
| 384 | CATTGTCTCCAGTCTCTTCG | −20.8 | −25.5 | 75.3 | −4.7 | 0 | −2.4 |
| | SEQ.ID.NO:10 | | | | | | |
| 57 | ACCGAGCTCAAACGGGTTCG | −20.7 | −25.9 | 69.1 | −3.1 | −2.1 | −9.8 |
| | SEQ.ID.NO:11 | | | | | | |
| 375 | CAGTCTCTTCGTTATGTTTT | −20.6 | −22.3 | 68.7 | −1.7 | 0 | −3 |
| | SEQ.ID.NO:12 | | | | | | |
| 56 | CCGAGCTCAAACGGGTTCGC | −20.5 | −27.5 | 72.4 | −5.6 | −1.2 | −10.1 |
| | SEQ.ID.NO:13 | | | | | | |
| 374 | AGTCTCTTCGTTATGTTTTG | −20 | −21.6 | 67.3 | −1.5 | 0 | −3 |
| | SEQ.ID.NO:14 | | | | | | |
| 60 | GATACCGAGCTCAAACGGGT | −19.7 | −24.9 | 68 | −3.1 | −2.1 | −9 |
| | SEQ.ID.NO:15 | | | | | | |
| 58 | TACCGAGCTCAAACGGGTTC | −19.6 | −24.8 | 68.5 | −3.1 | −2.1 | −9 |
| | SEQ.ID.NO:16 | | | | | | |
| 59 | ATACCGAGCTCAAACGGGTT | −19.2 | −24.4 | 67.1 | −3.1 | −2.1 | −8.7 |
| | SEQ.ID.NO:17 | | | | | | |
| 385 | ACATTGTCTCCAGTCTCTTC | −18.3 | −24.9 | 76.2 | −6.6 | 0 | −3.8 |
| | SEQ.ID.NO:18 | | | | | | |
| 61 | GGATACCGAGCTCAAACGGG | −17.6 | −24.9 | 67.4 | −6 | −1.2 | −9 |
| | SEQ.ID.NO:19 | | | | | | |
| 54 | GAGCTCAAACGGGTTCGCGC | −17.4 | −27.3 | 73 | −8.8 | −0.7 | −9.9 |
| | SEQ.ID.NO:20 | | | | | | |
| 372 | TCTCTTCGTTATGTTTTGTG | −17.2 | −21.6 | 66.9 | −4.4 | 0 | −3 |
| | SEQ.ID.NO:21 | | | | | | |
| 521 | TTAGAATCCAGCGAAGGTGA | −17.2 | −22.3 | 64.4 | −5.1 | 0 | −4.4 |
| | SEQ.ID.NO:22 | | | | | | |
| 53 | AGCTCAAACGGGTTCGCGCG | −17.1 | −27.5 | 71.7 | −8.8 | −0.7 | −11.2 |
| | SEQ.ID.NO:23 | | | | | | |
| 386 | CACATTGTCTCCAGTCTCTT | −17 | −25.2 | 75.5 | −8.2 | 0 | −4 |
| | SEQ.ID.NO:24 | | | | | | |
| 284 | ATCCTCCATGTCAAAAGCAT | −16.9 | −23.1 | 66 | −6.2 | 0 | −4.3 |
| | SEQ.ID.NO:25 | | | | | | |
| 387 | CCACATTGTCTCCAGTCTCT | −16.8 | −27.1 | 78.9 | −10.3 | 0 | −4 |
| | SEQ.ID.NO:26 | | | | | | |
| 128 | TTCAACTCGTACGCTGGAAC | −16.7 | −22.7 | 64.7 | −5.5 | 0 | −8.3 |
| | SEQ.ID.NO:27 | | | | | | |
| 519 | AGAATCCAGCGAAGGTGAAT | −16.7 | −21.8 | 62.6 | −5.1 | 0 | −4.1 |
| | SEQ.ID.NO:28 | | | | | | |
| 371 | CTCTTCGTTATGTTTTGTGT | −16.5 | −22.4 | 68.7 | −5.9 | 0 | −2.4 |
| | SEQ.ID.NO:29 | | | | | | |
| 282 | CCTCCATGTCAAAAGCATGT | −16.4 | −23.9 | 67.6 | −5.5 | −2 | −5.3 |
| | SEQ.ID.NO:30 | | | | | | |
| 520 | TAGAATCCAGCGAAGGTGAA | −16.4 | −21.5 | 62.1 | −5.1 | 0 | −4.4 |
| | SEQ.ID.NO:31 | | | | | | |
| 283 | TCCTCCATGTCAAAAGCATG | −16.3 | −23.1 | 65.9 | −5.5 | −1.2 | 4.4 |
| | SEQ.ID.NO:32 | | | | | | |
| 55 | CGAGCTCAAACGGGTTCGCG | −16.1 | −26.3 | 69.2 | −9 | −0.7 | −10.1 |
| | SEQ.ID.NO:33 | | | | | | |
| 588 | TGCACCACGATGTTTCAACA | −16.1 | −23.7 | 66.6 | −7.6 | 0 | −5.5 |
| | SEQ.ID.NO:34 | | | | | | |
| 64 | CTAGGATACCGAGCTCAAAC | −16 | −22.3 | 63.9 | −5.6 | −0.3 | −8.8 |
| | SEQ.ID.NO:35 | | | | | | |
| 65 | ACTAGGATACCGAGCTCAAA | −16 | −22.3 | 63.9 | −5.6 | −0.3 | −8.8 |
| | SEQ.ID.NO:36 | | | | | | |
| 127 | TCAACTCGTACGCTGGAACT | −16 | −23.5 | 66.1 | −7 | 0 | −8.3 |
| | SEQ.ID.NO:37 | | | | | | |
| 124 | ACTCGTACGCTGGAACTGGA | −15.7 | −24.9 | 69.2 | −8.7 | 0 | −8.3 |
| | SEQ.ID.NO:38 | | | | | | |
| 285 | AATCCTCCATGTCAAAAGCA | −15.7 | −22.4 | 64 | −6.7 | 0 | −4.3 |
| | SEQ.ID.NO:39 | | | | | | |
| 522 | TTTAGAATCCAGCGAAGGTG | −15.5 | −21.8 | 63.5 | −6.3 | 0 | −4.4 |
| | SEQ.ID.NO:40 | | | | | | |
| 517 | AATCCAGCGAAGGTGAATCA | −15.4 | −22.3 | 63.6 | −6.9 | 0 | −4.4 |
| | SEQ.ID.NO:41 | | | | | | |
| 132 | TCCATTCAACTCGTACGCTG | −15.3 | −24.5 | 68.5 | −8.7 | 0 | −8.3 |
| | SEQ.ID.NO:42 | | | | | | |
| 513 | CAGCGAAGGTGAATCAGACA | −15.3 | −22.1 | 63.8 | −6.8 | 0 | −4.5 |
| | SEQ.ID.NO:43 | | | | | | |
| 505 | GTGAATCAGACAGAGGTGGT | −15.2 | −23.1 | 69.4 | −7.9 | 0 | −5.2 |
| | SEQ.ID.NO:44 | | | | | | |
| 511 | GCGAAGGTGAATCAGACAGA | −15.2 | −22 | 63.9 | −6.8 | 0 | −4.5 |
| | SEQ.ID.NO:45 | | | | | | |
| 129 | ATTCAACTCGTACGCTGGAA | −15 | −22.5 | 64.1 | −7 | 0 | −8.3 |
| | SEQ.ID.NO:46 | | | | | | |
| 388 | CCCACATTGTCTCCAGTCTC | −15 | −28.2 | 80.6 | −13.2 | 0 | −4 |
| | SEQ.ID.NO:47 | | | | | | |
| 518 | GAATCCAGCGAAGGTGAATC | −15 | −22.2 | 63.7 | −7.2 | 0 | −4.4 |
| | SEQ.ID.NO:48 | | | | | | |
| 587 | GCACCACGATGTTTCAACAA | −14.9 | −23 | 64.7 | −7.6 | −0.2 | −5.9 |
| | SEQ.ID.NO:49 | | | | | | |
| 123 | CTCGTACGCTGGAACTGGAA | −14.8 | −24 | 66.6 | −8.7 | 0 | −8.3 |
| | SEQ.ID.NO:50 | | | | | | |
| 506 | GGTGAATCAGACAGAGGTGG | −14.8 | −23.1 | 68.6 | −8.3 | 0 | −5.2 |
| | SEQ.ID.NO:51 | | | | | | |
| 52 | GCTCAAACGGGTTCGCGCGG | −14.7 | −28.7 | 73.7 | −12.2 | −0.7 | −11.8 |
| | SEQ.ID.NO:52 | | | | | | |
| 62 | AGGATACCGAGCTCAAACGG | −14.7 | −23.7 | 65.3 | −7.3 | −1.7 | −8.9 |
| | SEQ.ID.NO:53 | | | | | | |
| 516 | ATCCAGCGAAGGTGAATCAG | −14.7 | −23 | 65.9 | −8.3 | 0 | −4.5 |
| | SEQ.ID.NO:54 | | | | | | |
| 133 | ATCCATTCAACTCGTACGCT | −14.6 | −24.5 | 68.7 | −9.4 | 0 | −8.3 |
| | SEQ.ID.NO:55 | | | | | | |
| 512 | AGCGAAGGTGAATCAGACAG | −14.6 | −21.4 | 62.9 | −6.8 | 0 | −4.5 |
| | SEQ.ID.NO:56 | | | | | | |
| 215 | ATTTCGATGATCTTTAACAT | −14.5 | −18 | 56.1 | −3.5 | 0 | −4.9 |
| | SEQ.ID.NO:57 | | | | | | |
| 527 | CCACATTTAGAATCCAGCGA | −14.5 | −23.7 | 66.2 | −9.2 | 0 | −4.1 |
| | SEQ.ID.NO:58 | | | | | | |
| 291 | CTCCCAAATCCTCCATGTCA | −14.4 | −27.3 | 74.3 | −12.9 | 0 | −4.3 |
| | SEQ.ID.NO:59 | | | | | | |
| 592 | AATGTGCACCACGATGTTTC | −14.3 | −23.3 | 66.7 | −7.6 | −1.3 | −8.8 |
| | SEQ.ID.NO:60 | | | | | | |
| 214 | TTTCGATGATCTTTAACATA | −14.2 | −17.7 | 55.6 | −3.5 | 0 | −4.9 |
| | SEQ.ID.NO:61 | | | | | | |
| 216 | TATTTCGATGATCTTTAACA | −14.2 | −17.7 | 55.6 | −3.5 | 0 | 4.9 |
| | SEQ.ID.NO:62 | | | | | | |
| 130 | CATTCAACTCGTACGCTGGA | −14.1 | −23.9 | 67.3 | −9.3 | 0 | −8.3 |
| | SEQ.ID.NO:63 | | | | | | |
| 281 | CTCCATGTCAAAAGCATGTA | −14.1 | −21.6 | 63.4 | −5.5 | −2 | 5.3 |
| | SEQ.ID.NO:64 | | | | | | |
| 528 | ACCACATTTAGAATCCAGCG | −14.1 | −23.3 | 65.6 | −9.2 | 0 | −4.1 |
| | SEQ.ID.NO:65 | | | | | | |
| 320 | CCTCCAACAACGGCAGTACA | −14 | −26 | 70.1 | −12 | 0 | −5.3 |
| | SEQ.ID.NO:66 | | | | | | |
| 523 | ATTTAGAATCCAGCGAAGGT | −14 | −21.8 | 63.5 | −7.8 | 0 | −4.4 |
| | SEQ.ID.NO:67 | | | | | | |
| 122 | CGTACGCTGGAACTGGAAG | −13.9 | −23.1 | 65.1 | −8.7 | 0 | −8.3 |
| | SEQ.ID.NO:68 | | | | | | |
| 279 | CCATGTCAAAAGCATGTACT | −13.9 | −21.4 | 62.6 | −5.5 | −2 | −5.5 |
| | SEQ.ID.NO:69 | | | | | | |
| 504 | TGAATCAGACAGAGGTGGTA | −13.8 | −21.6 | 65.4 | −7.8 | 0 | 3.9 |
| | SEQ.ID.NO:70 | | | | | | |
| 286 | AAATCCTCCATGTCAAAAGC | −13.7 | −21 | 60.9 | −7.3 | 0 | −4.3 |
| | SEQ.ID.NO:71 | | | | | | |
| 290 | TCCCAAATCCTCCATGTCAA | −13.7 | −2.5.7 | 70.2 | −12 | 0 | −4.3 |
| | SEQ.ID.NO:72 | | | | | | |
| 515 | TCCAGCGAAGGTGAATCAGA | −13.7 | −23.6 | 67.2 | −9.9 | 0 | −4.5 |
| | SEQ.ID.NO:73 | | | | | | |
| 31 | CGGCGAGAGTAAAGGTGCCA | −13.6 | −26.2 | 70.9 | −10.4 | −2.2 | −6.2 |
| | SEQ.ID.NO:74 | | | | | | |
| 389 | GCCCACATTGTCTCCAGTCT | −13.6 | −29.6 | 83.3 | −16 | 0 | −4 |
| | SEQ.ID.NO:75 | | | | | | |
| 507 | AGGTGAATCAGACAGAGGTG | −13.6 | −21.9 | 66.2 | −8.3 | 0 | −5.2 |
| | SEQ.ID.NO:76 | | | | | | |
| 591 | ATGTGCACCACGATGTTTCA | −13.6 | −24.7 | 70 | −9.7 | −1.3 | −8.8 |
| | SEQ.ID.NO:77 | | | | | | |
| 63 | TAGGATACCGAGCTCAAACG | −13.5 | −22.2 | 62.5 | −8 | −0.3 | −8.8 |
| | SEQ.ID.NO:78 | | | | | | |
| 213 | TTCGATGATCTTTAACATAA | −13.4 | −16.9 | 53.5 | −3.5 | 0 | −4.9 |
| | SEQ.ID.NO:79 | | | | | | |
| 280 | TCCATGTCAAAAGCATGTAC | −13.4 | −20.9 | 62.1 | −5.5 | −2 | −5.3 |
| | SEQ.ID.NO:80 | | | | | | |
| 510 | CGAAGGTGAATCAGACAGAG | −13.4 | −20.2 | 60.2 | −6.8 | 0 | −4.5 |
| | SEQ.ID.NO:81 | | | | | | |
| 66 | CACTAGGATACCGAGCTCAA | −13.3 | −23.7 | 67.1 | −9.8 | 0.5 | −8.8 |
| | SEQ.ID.NO:82 | | | | | | |
| 126 | CAACTCGTACGCTGGAACTG | −13.3 | −23.1 | 64.7 | −9.3 | 0 | 7.7 |
| | SEQ.ID.NO:83 | | | | | | |
| 370 | TCTTCGTTATGTTTTGTGTG | −13.3 | −21.5 | 66.5 | −8.2 | 0 | −3 |
| | SEQ.ID.NO:84 | | | | | | |
| 593 | AAATGTGCACCACGATGTTT | −13.2 | −22.2 | 63.3 | −7.6 | −1.3 | −8.8 |
| | SEQ.ID.NO:85 | | | | | | |
| 500 | TCAGACAGAGGTGGTAGTCA | −13.1 | −24 | 73.4 | −9.6 | −1.2 | −4.6 |
| | SEQ.ID.NO:86 | | | | | | |
| 617 | TTTTTTTTTTTTTTTTGTTT | −13.1 | −16.9 | 56.1 | −3.8 | 0 | 0 |
| | SEQ.ID.NO:87 | | | | | | |
| 32 | CCGGCGAGAGTAAAGGTGCC | −13 | −27.5 | 73.2 | −13.3 | −1.1 | −5.3 |
| | SEQ.ID.NO:88 | | | | | | |
| 89 | GCCGTCGCTTGCAAAGGCGT | −13 | −29.9 | 77.5 | −13.9 | −3 | −11.6 |
| | SEQ.ID.NO:89 | | | | | | |
| 121 | CGTACGCTGGAACTGGAAGT | −13 | −23.9 | 66.6 | −10 | −0.8 | −8.8 |
| | SEQ.ID.NO:90 | | | | | | |
| 275 | GTCAAAAGCATGTACTATCT | −13 | −19.7 | 60.5 | −6.7 | 0 | −5 |
| | SEQ.ID.NO:91 | | | | | | |
| 529 | TACCACATTTAGAATCCAGC | −13 | −22.2 | 64.7 | −9.2 | 0 | −2.8 |
| | SEQ.ID.NO:92 | | | | | | |
| 67 | GCACTAGGATACCGAGCTCA | −12.9 | −26.2 | 73.4 | −12.6 | −0.3 | −8.8 |
| | SEQ.ID.NO:93 | | | | | | |
| 277 | ATGTCAAAAGCATGTACTAT | −12.9 | −18.4 | 57.1 | −5.5 | 0 | −5.5 |
| | SEQ.ID.NO:94 | | | | | | |
| 69 | GTGCACTAGGATACCGAGCT | −12.8 | −26.3 | 73.9 | −12.7 | −0.3 | −8.9 |
| | SEQ.ID.NO:95 | | | | | | |
| 509 | GAAGGTGAATCAGACAGAGG | −12.8 | −20.6 | 62.2 | −7.8 | 0 | −4 |
| | SEQ.ID.NO:96 | | | | | | |
| 319 | CTCCAACAACGGCAGTACAC | −12.7 | −24.2 | 67.3 | −11.5 | 0 | −5.3 |
| | SEQ.ID.NO:97 | | | | | | |
| 499 | CAGACAGAGGTGGTAGTCAT | −12.7 | −23.6 | 71.5 | −9.6 | −1.2 | −4.6 |
| | SEQ.ID.NO:98 | | | | | | |
| 212 | TCGATGATCTTTAACATAAA | −12.6 | −16.1 | 51.5 | −3.5 | 0 | −4.2 |
| | SEQ.ID.NO:99 | | | | | | |
| 276 | TGTCAAAAGCATGTACTATC | −12.6 | −18.8 | 58.4 | −6.2 | 0 | −5 |
| | SEQ.ID.NO:100 | | | | | | |
| 289 | CCCAAATCCTCCATGTCAAA | −12.6 | −24.6 | 66.8 | −12 | 0 | −4.3 |
| | SEQ.ID.NO:101 | | | | | | |
| 492 | AGGTGGTAGTCATTCTAATT | −12.6 | −21.3 | 66.3 | −8.7 | 0 | −3.8 |
| | SEQ.ID.NO:102 | | | | | | |
| 46 | ACGGGTTCGCGCGGCCGGCG | −12.5 | −34.7 | 82.3 | −17.7 | −3.6 | −17 |
| | SEQ.ID.NO:103 | | | | | | |
| 217 | TTATTTCGATGATCTTTAAC | −12.5 | −17.1 | 54.6 | −4.6 | 0 | −4.9 |
| | SEQ.ID.NO:104 | | | | | | |
| 269 | AGCATGTACTATCTTGGGGT | −12.4 | −24.4 | 72.9 | −12 | 0 | −5.3 |
| | SEQ.ID.NO:105 | | | | | | |
| 491 | GGTGGTAGTCATTCTAATTA | −12.3 | −21 | 65.5 | −8.7 | 0 | −3.8 |
| | SEQ.ID.NO:106 | | | | | | |
| 539 | GTTTGCAATATACCACATTT | −12.3 | −20.6 | 61.5 | −8.3 | 0 | −7.1 |
| | SEQ.ID.NO:107 | | | | | | |
| 595 | ACAAATGTGCACCACGATGT | −12.3 | −22.9 | 64.2 | −9.2 | −1.3 | −8.8 |
| | SEQ.ID.NO:108 | | | | | | |
| 50 | TCAAACGGGTTCGCGCGGCC | −12.2 | −29.8 | 75.1 | −15.8 | 0.2 | −11.8 |
| | SEQ.ID.NO:109 | | | | | | |
| 590 | TGTGCACCACGATGTTTCAA | −12.2 | −24 | 67.9 | −10.4 | −1.3 | −8.8 |
| | SEQ.ID.NO:110 | | | | | | |
| 45 | CGGGTTCGCGCGGCCGGCGA | −12.1 | −35.1 | 82.8 | −17.7 | −2.9 | −18.7 |
| | SEQ.ID.NO:111 | | | | | | |
| 498 | AGACAGAGGTGGTAGTCATT | −12.1 | −23 | 70.7 | −9.6 | −1.2 | −4.6 |
| | SEQ.ID.NO:112 | | | | | | |
| 134 | GATCCATTCAACTCGTACGC | −12 | −24.2 | 68.1 | −11.7 | 0 | −8.3 |
| | SEQ.ID.NO:113 | | | | | | |
| 278 | CATGTCAAAAGCATGTACTA | −12 | −19.1 | 58.4 | −5.5 | −1.5 | −5.5 |
| | SEQ.ID.NO:114 | | | | | | |
| 369 | CTTCGTTATGTTTTGTGTGA | −12 | −21.7 | 66.3 | −9.7 | 0 | −3 |
| | SEQ.ID.NO:115 | | | | | | |
| 252 | GGTTGTCTTTCTGGATGTGA | −11.9 | −24.1 | 73.3 | −12.2 | 0 | −2.6 |
| | SEQ.ID.NO:116 | | | | | | |
| 51 | CTCAAACGGGTTCGCGCGGC | −11.8 | −28.7 | 73.7 | −15.1 | −0.7 | −11.8 |
| | SEQ.ID.NO:117 | | | | | | |
| 70 | TGTGCACTAGGATACCGAGC | −11.8 | −25.4 | 71.8 | −12.7 | −0.3 | −9.7 |
| | SEQ.ID.NO:118 | | | | | | |
| 536 | TGCAATATACCACATTTAGA | −11.8 | −19.5 | 58.8 | −7.7 | 0 | −4.7 |
| | SEQ.ID.NO:119 | | | | | | |
| 594 | CAAATGTGCACCACGATGTT | −11.8 | −22.8 | 64.1 | −10.2 | −0.6 | −8.1 |
| | SEQ.ID.NO:120 | | | | | | |
| 321 | ACCTCCAACAACGGCAGTAC | −11.7 | −25.5 | 69.6 | −13.8 | 0 | −5.1 |
| | SEQ.ID.NO:121 | | | | | | |
| 412 | TCTTTTTTCTTGATGATCAG | −11.7 | −19.5 | 61.8 | −7.8 | 0 | −6.8 |
| | SEQ.ID.NO:122 | | | | | | |
| 503 | GAATCAGACAGAGGTGGTAG | −11.7 | −21.6 | 65.7 | −9.9 | 0 | −2.8 |
| | SEQ.ID.NO:123 | | | | | | |
| 218 | TTTATTTCGATGATCTTTAA | −11.6 | −17 | 54.4 | −5.4 | 0 | 4.9 |
| | SEQ.ID.NO:124 | | | | | | |
| 47 | AACGGGTTCGCGCGGCCGGC | −11.5 | −33.2 | 80.5 | −17.7 | −1.9 | −16.1 |
| | SEQ.ID.NO:125 | | | | | | |
| 103 | GTCAGACTCATGGCGCCGTC | −11.5 | −29.1 | 80.2 | −15.7 | 0 | −12 |
| | SEQ.ID.NO:126 | | | | | | |
| 211 | CGATGATCTTTAACATAAAA | −11.5 | −15 | 48.9 | −3.5 | 0 | −4.9 |
| | SEQ.ID.NO:127 | | | | | | |
| 251 | GTTGTCTTTCTGGATGTGAA | −11.5 | −22.2 | 68 | −10.7 | 0 | −3.4 |
| | SEQ.ID.NO:128 | | | | | | |
| 29 | GCGAGAGTAAAGGTGCCAGC | −11.4 | −26 | 72.8 | −14.6 | 0 | −6.2 |
| | SEQ.ID.NO:129 | | | | | | |
| 131 | CCATTCAACTCGTACGCTGG | −11.3 | −25.3 | 69.5 | −13.5 | 0 | −8.3 |
| | SEQ.ID.NO:130 | | | | | | |
| 274 | TCAAAAGCATGTACTATCTT | −11.3 | −18.6 | 57.8 | −7.3 | 0 | −5 |
| | SEQ.ID.NO:131 | | | | | | |
| 597 | AAACAAATGTGCACCACGAT | −11.3 | −20.3 | 58 | −7.6 | −1.3 | −8.8 |
| | SEQ.ID.NO:132 | | | | | | |
| 508 | AAGGTGAATCAGACAGAGGT | −11.2 | −21.2 | 64.1 | −10 | 0 | −4.5 |
| | SEQ.ID.NO:133 | | | | | | |
| 530 | ATACCACATTTAGAATCCAG | −11.2 | −20.4 | 60.7 | −9.2 | 0 | −2.4 |
| | SEQ.ID.NO:134 | | | | | | |
| 33 | GCCGGCGAGAGTAAAGGTGC | −11.1 | −27.3 | 73.9 | −14.6 | 0 | −11.4 |
| | SEQ.ID.NO:135 | | | | | | |
| 41 | TTCGCGCGGCCGGCGAGAGT | −11.1 | −32.5 | 80.7 | −15.8 | −3.7 | −19.4 |
| | SEQ.ID.NO:136 | | | | | | |
| 42 | GTTCGCGCGGCCGGCGAGAG | −11.1 | −32.5 | 80.7 | −15.8 | −3.7 | −19.4 |
| | SEQ.ID.NO:137 | | | | | | |
| 205 | TCTTTAACATAAAATCTTTT | −11.1 | −14.6 | 49.1 | −3.5 | 0 | −3.3 |
| | SEQ.ID.NO:138 | | | | | | |
| 268 | GCATGTACTATCTTGGGGTT | −11.1 | −24.5 | 73 | −13.4 | 0 | −5.3 |
| | SEQ.ID.NO:139 | | | | | | |
| 535 | GCAATATACCACATTTAGAA | −11.1 | −18.8 | 57 | −7.7 | 0 | −3.4 |
| | SEQ.ID.NO:140 | | | | | | |
| 616 | TTTTTTTTTTTTTTTGTTTA | −11.1 | −16.5 | 55.2 | −5.4 | 0 | −0.2 |
| | SEQ.ID.NO:141 | | | | | | |
| 39 | CGCGCGGCCGGCGAGAGTAA | −11 | −31 | 76.2 | −15.8 | −2.9 | −16.6 |
| | SEQ.ID.NO:142 | | | | | | |
| 40 | TCGCGCGGCCGGCGAGAGTA | −11 | −32.1 | 79.8 | −15.8 | −3.4 | −18.8 |
| | SEQ.ID.NO:143 | | | | | | |
| 102 | TCAGACTCATGGCGCCGTCG | −11 | −28.7 | 76.5 | −15.7 | −0.8 | −12.1 |
| | SEQ.ID.NO:144 | | | | | | |
| 135 | CGATCCATTCAACTCGTACG | −11 | −23.2 | 64.4 | −11.7 | 0 | −8 |
| | SEQ.ID.NO:145 | | | | | | |
| 206 | ATCTTTAACATAAAATCTTT | −11 | −14.5 | 48.9 | −3.5 | 0 | −2.7 |
| | SEQ.ID.NO:146 | | | | | | |
| 598 | TAAACAAATGTGCACCACGA | −11 | −20 | 57.5 | −7.6 | −1.3 | −8.8 |
| | SEQ.ID.NO:147 | | | | | | |
| 207 | GATCTTTAACATAAAATCTT | −10.9 | −15 | 49.8 | −3.5 | −0.3 | −4.1 |
| | SEQ.ID.NO:148 | | | | | | |
| 413 | TTCTTTTTTCTTGATGATCA | −10.9 | −19.6 | 61.9 | −8.7 | 0 | −6.5 |
| | SEQ.ID.NO:149 | | | | | | |
| 420 | TTTAAGTTTCTTTTTTCTTG | −10.9 | −17.8 | 58.2 | −6.9 | 0 | −2.7 |
| | SEQ.ID.NO:150 | | | | | | |
| 531 | TATACCACATTTAGAATCCA | −10.9 | −20.1 | 60 | −9.2 | 0 | −2.4 |
| | SEQ.ID.NO:151 | | | | | | |
| 480 | TTCTAATTAAACAATCAGGT | −10.8 | −16.5 | 53 | −5.7 | 0 | −3.8 |
| | SEQ.ID.NO:152 | | | | | | |
| 68 | TGCACTAGGATACCGAGCTC | −10.7 | −25.5 | 72.2 | −14.2 | −0.3 | −7.5 |
| | SEQ.ID.NO:153 | | | | | | |
| 204 | CTTTAACATAAAATCTTTTG | −10.7 | −14.2 | 48.1 | −3.5 | 0 | −3.7 |
| | SEQ.ID.NO:154 | | | | | | |
| 210 | GATGATCTTTAACATAAAAT | −10.7 | −14.2 | 47.8 | −3.5 | 0 | −4.9 |
| | SEQ.ID.NO:155 | | | | | | |
| 292 | TCTCCCAAATCCTCCATGTC | −10.7 | −27 | 74.8 | −16.3 | 0 | −4.3 |
| | SEQ.ID.NO:156 | | | | | | |
| 540 | AGTTTGCAATATACCACATT | −10.7 | −20.5 | 61.4 | −9.8 | 0 | −7.1 |
| | SEQ.ID.NO:157 | | | | | | |
| 514 | CCAGCGAAGGTGAATCAGAC | −10.6 | −23.4 | 66.2 | −12.8 | 0 | −4.5 |
| | SEQ.ID.NO:158 | | | | | | |
| 524 | CATTTAGAATCCAGCGAAGG | −10.6 | −21.3 | 61.7 | −10.7 | 0 | −4.1 |
| | SEQ.ID.NO:159 | | | | | | |
| 38 | GCGCGGCCGGCGAGAGTAAA | −10.5 | −29.5 | 74.3 | −15.8 | −2.9 | −14.1 |
| | SEQ.ID.NO:160 | | | | | | |
| 203 | TTTAACATAAAATCTTTTGT | −10.5 | −14.5 | 48.8 | −3.5 | −0.1 | −3.7 |
| | SEQ.ID.NO:161 | | | | | | |
| 219 | CTTTATTTCGATGATCTTTA | −10.5 | −18.6 | 58.3 | −8.1 | 0 | −4.9 |
| | SEQ.ID.NO:162 | | | | | | |
| 270 | AAGCATGTACTATCTTGGGG | −10.5 | −22.5 | 67 | −12 | 0 | −5 |
| | SEQ.ID.NO:163 | | | | | | |
| 390 | GGCCCACATTGTCTCCAGTC | −10.5 | −29.9 | 84 | −19.4 | 0 | −5.6 |
| | SEQ.ID.NO:164 | | | | | | |
| 411 | CTTTTTTCTTGATGATCAGA | −10.5 | −19.7 | 61.7 | −9.2 | 0 | −6.8 |
| | SEQ.ID.NO:165 | | | | | | |
| 602 | TGTTTAAACAAATGTGCACC | −10.5 | −19.1 | 57.3 | −7.6 | 0 | −9.9 |
| | SEQ.ID.NO:166 | | | | | | |
| 28 | CGAGAGTAAAGGTGCCAGCG | −10.4 | −25 | 68.8 | −14.6 | 0 | −6.2 |
| | SEQ.ID.NO:167 | | | | | | |
| 30 | GGCGAGAGTAAAGGTGCCAG | −10.4 | −25.4 | 71.2 | −13.3 | −1.7 | −6.2 |
| | SEQ.ID.NO:168 | | | | | | |
| 120 | GTACGCTGGAACTGGAAGTC | −10.4 | −23.5 | 67.9 | −12 | −1 | −6.4 |
| | SEQ.ID.NO:169 | | | | | | |
| 490 | GTGGTAGTCATTCTAATTAA | −10.4 | −19.1 | 60.4 | −8.7 | 0 | −3.8 |
| | SEQ.ID.NO:170 | | | | | | |
| 3 | CTAAAGCACCGACTCCGCGA | −10.3 | −26.8 | 69.3 | −16 | −0.2 | −7.2 |
| | SEQ.ID.NO:171 | | | | | | |
| 208 | TGATCTTTAACATAAAATCT | −10.3 | −14.9 | 49.5 | −3.5 | −1 | −4.9 |
| | SEQ.ID.NO:172 | | | | | | |
| 287 | CAAATCCTCCATGTCAAAAG | −10.3 | −19.9 | 58.4 | −9.6 | 0 | −4.3 |
| | SEQ.ID.NO:173 | | | | | | |
| 34 | GGCCGGCGAGAGTAAAGGTG | −10.2 | −26.7 | 72.3 | −14.6 | 0 | −12 |
| | SEQ.ID.NO:174 | | | | | | |
| 160 | GTCCCAGCAGCAATGGTAAC | −10.2 | −26.2 | 73.4 | −14.6 | −1.3 | −6 |
| | SEQ.ID.NO:175 | | | | | | |
| 532 | ATATACCACATTTAGAATCC | −10.2 | −19.4 | 58.8 | −9.2 | 0 | −2.4 |
| | SEQ.ID.NO:176 | | | | | | |
| 589 | GTGCACCACGATGTTTCAAC | −10.2 | −24.2 | 68.5 | −13.2 | −0.6 | −7.8 |
| | SEQ.ID.NO:177 | | | | | | |
| 263 | TACTATCTTGGGGTTGTCTT | −10.1 | −23.4 | 71.4 | −13.3 | 0 | −2.4 |
| | SEQ.ID.NO:178 | | | | | | |
| 119 | TACGCTGGAACTGGAAGTCA | −10 | −23 | 65.9 | −11.9 | −1 | −5.1 |
| | SEQ.ID.NO:179 | | | | | | |
| 209 | ATGATCTTTAACATAAAATC | −10 | −14 | 47.7 | −3.5 | −0.2 | −4.9 |
| | SEQ.ID.NO:180 | | | | | | |
| 502 | AATCAGACAGAGGTGGTAGT | −10 | −22.2 | 67.8 | −12.2 | 0 | −2.9 |
| | SEQ.ID.NO:181 | | | | | | |
| 159 | TCCCAGCAGCAATGGTAACT | −9.9 | −25.9 | 72.1 | −14.6 | −1.3 | 5.9 |
| | SEQ.ID.NO:182 | | | | | | |
| 419 | TTAAGTTTCTTTTTTCTTGA | −9.9 | −18.3 | 59.2 | −8.4 | 0 | −2.7 |
| | SEQ.ID.NO:183 | | | | | | |
| 538 | TTTGCAATATACCACATTTA | −9.9 | −19.1 | 58 | −9.2 | 0 | −7.1 |
| | SEQ.ID.NO:184 | | | | | | |
| 24 | AGTAAAGGTGCCAGCGGCGT | −9.8 | −28 | 75.6 | −15.8 | −2.4 | −10.6 |
| | SEQ.ID.NO:185 | | | | | | |
| 118 | ACGCTGGAACTGGAAGTCAG | −9.8 | −23.3 | 66.7 | −11.9 | −1.5 | −5.6 |
| | SEQ.ID.NO:186 | | | | | | |
| 136 | GCGATCCATTCAACTCGTAC | −9.8 | −24.2 | 68.1 | −13.7 | −0.4 | −3.9 |
| | SEQ.ID.NO:187 | | | | | | |
| 324 | TGGACCTCCAACAACGGCAG | −9.8 | −26.2 | 70 | −15.9 | −0.2 | −6.2 |
| | SEQ.ID.NO:188 | | | | | | |
| 4 | ACTAAAGCACCGACTCCGCG | −9.6 | −26.4 | 68.7 | −16 | −0.6 | −6.6 |
| | SEQ.ID.140:189 | | | | | | |
| 125 | AACTCGTACGCTGGAACTGG | −9.6 | −23.6 | 65.9 | −13.5 | 0 | −8.3 |
| | SEQ.ID.NO:190 | | | | | | |
| 258 | TCTTGGGGTTGTCTTTCTGG | −9.6 | −25.5 | 77 | −15.9 | 0 | 1.5 |
| | SEQ.ID.NO:191 | | | | | | |
| 481 | ATTCTAATTAAACAATCAGG | −9.6 | −15.3 | 50.3 | −5.7 | 0 | −3.8 |
| | SEQ.ID.NO:192 | | | | | | |
| 23 | GTAAAGGTGCCAGCGGCGTA | −9.5 | −27.7 | 74.8 | −15.8 | −2.4 | −10.6 |
| | SEQ.ID.NO:193 | | | | | | |
| 73 | GCGTGTGCACTAGGATACCG | −9.5 | −26.8 | 73.4 | −15.9 | −1.2 | −9.7 |
| | SEQ.ID.NO:194 | | | | | | |
| 541 | CAGTTTGCAATATACCACAT | −9.5 | −21.1 | 62.3 | −11.6 | 0 | −7.1 |
| | SEQ.ID.NO:195 | | | | | | |
| 421 | ATTTAAGTTTCTTTTTTCTT | −9.4 | −17.8 | 58.2 | −8.4 | 0 | −2.7 |
| | SEQ.ID.NO:196 | | | | | | |
| 495 | CAGAGGTGGTAGTCATTCTA | −9.4 | −23.2 | 71.6 | −13.8 | 0 | −3.8 |
| | SEQ.ID.NO:197 | | | | | | |
| 596 | AACAAATGTGCACCACGATG | −9.4 | −21 | 59.6 | −10.2 | −1.3 | −8.8 |
| | SEQ.ID.NO:198 | | | | | | |
| 615 | TTTTTTTTTTTTTTGTTTAA | −9.4 | −15.7 | 52.8 | −6.3 | 0 | −2.2 |
| | SEQ.ID.NO:199 | | | | | | |
| 37 | CGCGGCCGGCGAGAGTAAAG | −9.3 | −27.7 | 70.9 | −15.8 | −2.2 | −13 |
| | SEQ.ID.NO:200 | | | | | | |
| 99 | GACTCATGGCGCCGTCGCTT | −9.3 | −30.4 | 79.8 | −17.8 | −3.3 | −12.1 |
| | SEQ.ID.NO:201 | | | | | | |
| 117 | CGCTGGAACTGGAAGTCAGA | −9.3 | −23.7 | 67.4 | −12.5 | −1.9 | −5.8 |
| | SEQ.ID.NO:202 | | | | | | |
| 334 | GGGAACTTTTTGGACCTCCA | −9.3 | −25.8 | 72.2 | −15.6 | −0.7 | −6.5 |
| | SEQ.ID.NO:203 | | | | | | |
| 501 | ATCAGACAGAGGTGGTAGTC | −9.3 | −23.3 | 72.1 | −13.5 | −0.1 | −4.4 |
| | SEQ.ID.NO:204 | | | | | | |
| 534 | CAATATACCACATTTAGAAT | −9.3 | −17 | 53.3 | −7.7 | 0 | −2.7 |
| | SEQ.ID.NO:205 | | | | | | |
| 25 | GAGTAAAGGTGCCAGCGGCG | −9.2 | −27.4 | 73.6 | −15.8 | −2.4 | −10.6 |
| | SEQ.ID.NO:206 | | | | | | |
| 220 | GCTTTATTTCGATGATCTTT | −9.2 | −20.7 | 63 | −11.5 | 0 | −4.9 |
| | SEQ.ID.NO:207 | | | | | | |
| 325 | TTGGACCTCCAACAACGGCA | −9.2 | −26.3 | 70.1 | −15.9 | −1.1 | −8.1 |
| | SEQ.ID.NO:208 | | | | | | |
| 487 | GTAGTCATTCTAATTAAACA | −9.2 | −16.9 | 54.6 | −7.7 | 0 | −4.4 |
| | SEQ.ID.NO:209 | | | | | | |
| 586 | CACCACGATGTTTCAACAAG | −9.2 | −21.2 | 61.1 | −11.5 | −0.2 | −5.9 |
| | SEQ.ID.NO:210 | | | | | | |
| 250 | TTGTCTTTCTGGATGTGAAG | −9.1 | −21 | 64.8 | −11.9 | 0 | −3.4 |
| | SEQ.ID.NO:211 | | | | | | |
| 257 | CTTGGGGTTGTCTTTCTGGA | −9.1 | −25.7 | 76.6 | −16.6 | 0 | −2.8 |
| | SEQ.ID.NO:212 | | | | | | |
| 262 | ACTATCTTGGGGTTGTCTTT | −9.1 | −23.8 | 72.5 | −14.7 | 0 | −2.2 |
| | SEQ.ID.NO:213 | | | | | | |
| 264 | GTACTATCTTGGGGTTGTCT | −9.1 | −24.5 | 74.8 | −15.4 | 0 | −4 |
| | SEQ.ID.NO:214 | | | | | | |
| 35 | CGGCCGGCGAGAGTAAAGGT | −9 | −27.5 | 72.3 | −16.6 | −0.1 | −12 |
| | SEQ.ID.NO:215 | | | | | | |
| 288 | CCAAATCCTCCATGTCAAAA | −9 | −21.9 | 61.6 | −12.9 | 0 | −4.3 |
| | SEQ.ID.NO:216 | | | | | | |
| 318 | TCCAACAACGGCAGTACACA | −9 | −24 | 66.6 | −15 | 0 | −5.3 |
| | SEQ.ID.NO:217 | | | | | | |
| 335 | TGGGAACTTTTTGGACCTCC | −9 | −25.1 | 70.9 | −15.6 | −0.2 | −4.3 |
| | SEQ.ID.NO:218 | | | | | | |
| 479 | TCTAATTAAACAATCAGGTA | −9 | −16.1 | 52.1 | −7.1 | 0 | −3.8 |
| | SEQ.ID.NO:219 | | | | | | |
| 43 | GGTTCGCGCGGCCGGCGAGA | −8.9 | −33.7 | 82.6 | −19.2 | −3.5 | −19.4 |
| | SEQ.ID.NO:220 | | | | | | |
| 202 | TTAACATAAAATCTTTTGTA | −8.9 | −14.1 | 48 | −4.6 | −0.3 | −3.7 |
| | SEQ.ID.NO:221 | | | | | | |
| 293 | ATCTCCCAAATCCTCCATGT | −8.9 | −26.6 | 73.2 | −17.7 | 0 | −4.3 |
| | SEQ.ID.NO:222 | | | | | | |
| 393 | GAGGGCCCACATTGTCTCCA | −8.9 | −30.1 | 82.4 | −19.6 | 0 | −11.3 |
| | SEQ.ID.NO:223 | | | | | | |
| 5 | TACTAAAGCACCGACTCCGC | −8.8 | −25.3 | 68.1 | −16 | −0.2 | −4.4 |
| | SEQ.ID.NO:224 | | | | | | |
| 27 | GAGAGTAAAGGTGCCAGCGG | −8.8 | −25.4 | 71.2 | −16.6 | 0 | −6.2 |
| | SEQ.ID.NO:225 | | | | | | |
| 72 | CGTGTGCACTAGGATACCGA | −8.8 | −25.6 | 70.6 | −15.9 | −0.3 | −9.7 |
| | SEQ.ID.NO:226 | | | | | | |
| 315 | AACAACGGCAGTACACAGCT | −8.8 | −23.6 | 66.6 | −14 | −0.6 | −5.5 |
| | SEQ.ID.NO:227 | | | | | | |
| 322 | GACCTCCAACAACGGCAGTA | −8.8 | −25.9 | 70.3 | −17.1 | 0 | −5.1 |
| | SEQ.ID.NO:228 | | | | | | |
| 100 | AGACTCATGGCGCCGTCGCT | −8.7 | −30.3 | 79.7 | −18.3 | −3.3 | −12.1 |
| | SEQ.ID.NO:229 | | | | | | |
| 314 | ACAACGGCAGTACACAGCTT | −8.7 | −24.4 | 69 | −14.9 | −0.6 | −5.5 |
| | SEQ.ID.NO:230 | | | | | | |
| 414 | TTTCTTTTTTCTTGATGATC | −8.7 | −19 | 61 | −10.3 | 0 | −3.9 |
| | SEQ.ID.NO:231 | | | | | | |
| 271 | AAAGCATGTACTATCTTGGG | −8.6 | −20.6 | 62.2 | −12 | 0 | −5 |
| | SEQ.ID.NO:232 | | | | | | |
| 273 | CAAAAGCATGTACTATCTTG | −8.6 | −18.2 | 56.4 | −9.6 | 0 | −5 |
| | SEQ.ID.NO:233 | | | | | | |
| 493 | GAGGTGGTAGTCATTCTAAT | −8.6 | −21.8 | 67.4 | −13.2 | 0 | −3.8 |
| | SEQ.ID.NO:234 | | | | | | |
| 547 | AAGCTGCAGTTTGCAATATA | −8.6 | −21.1 | 63.2 | −10.3 | −2.2 | −9 |
| | SEQ.ID.NO:235 | | | | | | |
| 297 | CTTTATCTCCCAAATCCTCC | −8.5 | −25.5 | 71.1 | −17 | 0 | −1.8 |
| | SEQ.ID.NO:236 | | | | | | |
| 259 | ATCTTGGGGTTGTCTTTCTG | −8.4 | −24.3 | 74.1 | −15.9 | 0 | −1.5 |
| | SEQ.ID.NO:237 | | | | | | |
| 418 | TAAGTTTCTTTTTTCTTGAT | −8.4 | −18.2 | 58.8 | −9.8 | 0 | −2.7 |
| | SEQ.ID.NO:238 | | | | | | |
| 599 | TTAAACAAATGTGCACCACG | −8.4 | −19.5 | 56.6 | −9.7 | −1.3 | −8.8 |
| | SEQ.ID.NO:239 | | | | | | |
| 313 | CAACGGCAGTACACAGCTTT | −8.3 | −24.3 | 68.8 | −16 | 0.2 | −5.1 |
| | SEQ.ID.NO:240 | | | | | | |
| 392 | AGGGCCCACATTGTCTCCAG | −8.3 | −29.5 | 81.4 | −19.6 | 0 | −11.3 |
| | SEQ.ID.NO:241 | | | | | | |
| 494 | AGAGGTGGTAGTCATTCTAA | −8.3 | −21.8 | 67.7 | −13.5 | 0 | −3.7 |
| | SEQ.ID.NO:242 | | | | | | |
| 227 | TATCATAGCTTTATTTCGAT | −8.2 | −19.1 | 59.4 | −10.9 | 0 | 4.7 |
| | SEQ.ID.NO:243 | | | | | | |
| 316 | CAACAACGGCAGTACACAGC | −8.2 | −23.4 | 65.8 | −15.2 | 0 | −5.4 |
| | SEQ.ID.NO:244 | | | | | | |
| 394 | AGAGGGCCCACATTGTCTCC | −8.2 | −29.4 | 81.7 | −19.6 | 0 | −11.3 |
| | SEQ.ID.NO:245 | | | | | | |
| 472 | AAACAATCAGGTAACTTCAC | −8.2 | −17.8 | 55.5 | −8.7 | −0.8 | 3.7 |
| | SEQ.ID.NO:246 | | | | | | |
| 79 | GCAAAGGCGTGTGCACTAGG | −8.1 | −25.8 | 72.1 | −16 | −1.7 | −10.2 |
| | SEQ.ID.NO:247 | | | | | | |
| 152 | AGCAATGGTAACTGCTGCGA | −8.1 | −24.2 | 68.1 | −14.6 | −1.4 | −7 |
| | SEQ.ID.NO:248 | | | | | | |
| 260 | TATCTTGGGGTTGTCTTTCT | −8.1 | −24 | 73.7 | −15.9 | 0 | −1.5 |
| | SEQ.ID.NO:249 | | | | | | |
| 309 | GGCAGTACACAGCTTTATCT | −8.1 | −24.3 | 72.4 | −15.4 | −0.6 | −5.5 |
| | SEQ.ID.NO:250 | | | | | | |
| 496 | ACAGAGGTGGTAGTCATTCT | −8.1 | −23.7 | 72.9 | −15.6 | 0 | −3.7 |
| | SEQ.ID.NO:251 | | | | | | |
| 525 | ACATTTAGAATCCAGCGAAG | −8.1 | −20.3 | 59.9 | −12.2 | 0 | 4.1 |
| | SEQ.ID.NO:252 | | | | | | |
| 426 | TGTCCATTTAAGTTTCTTTT | −8 | −20.5 | 63.7 | −12.5 | 0 | −2.7 |
| | SEQ.ID.NO:253 | | | | | | |
| 546 | AGCTGCAGTTTGCAATATAC | −8 | −22 | 65.9 | −11.8 | −2.2 | −8.9 |
| | SEQ.ID.NO:254 | | | | | | |
| 585 | ACCACGATGTTTCAACAAGA | −8 | −21.1 | 61.1 | −12.6 | −0.2 | −5.9 |
| | SEQ.ID.NO:255 | | | | | | |
| 151 | GCAATGGTAACTGCTGCGAT | −7.9 | −24.2 | 67.8 | −15.5 | −0.6 | −6.4 |
| | SEQ.ID.NO:256 | | | | | | |
| 533 | AATATACCACATTTAGAATC | −7.9 | −16.7 | 53.2 | −8.8 | 0 | −2.7 |
| | SEQ.ID.NO:257 | | | | | | |
| 161 | TGTCCCAGCAGCAATGGTAA | −7.8 | −26 | 72.7 | −16.8 | −1.3 | −6 |
| | SEQ.ID.NO:258 | | | | | | |
| 162 | CTGTCCCAGCAGCAATGGTA | −7.8 | −27.6 | 77 | −18.4 | −1.3 | −6.6 |
| | SEQ.ID.NO:259 | | | | | | |
| 231 | GGTTTATCATAGCTTTATTT | −7.8 | −19.9 | 62.7 | −12.1 | 0 | −4.6 |
| | SEQ.ID.NO:260 | | | | | | |
| 296 | TTTATCTCCCAAATCCTCCA | −7.8 | −25.3 | 70.4 | −17.5 | 0 | −1.6 |
| | SEQ.ID.MO:261 | | | | | | |
| 603 | TTGTTTAAACAAATGTGCAC | −7.8 | −17.2 | 54 | −7.6 | −0.7 | −11.8 |
| | SEQ.ID.NO:262 | | | | | | |
| 71 | GTGTGCACTAGGATACCGAG | −7.7 | −24.8 | 70.9 | −16.2 | −0.3 | −9.7 |
| | SEQ.ID.NO:263 | | | | | | |
| 232 | AGGTTTATCATAGCTTTATT | −7.7 | −19.8 | 62.5 | −12.1 | 0 | −4.6 |
| | SEQ.ID.NO:264 | | | | | | |
| 368 | TTCGTTATGTTTTGTGTGAG | −7.7 | −20.8 | 64.5 | −13.1 | 0 | −3 |
| | SEQ.ID.NO:265 | | | | | | |
| 475 | ATTAAACAATCAGGTAACTT | −7.7 | −16.3 | 52.3 | −7.7 | −0.8 | −4.5 |
| | SEQ.ID.NO:266 | | | | | | |
| 482 | CATTCTAATTAAACAATCAG | −7.7 | −14.8 | 49.1 | −7.1 | 0 | −3.6 |
| | SEQ.ID.NO:267 | | | | | | |
| 600 | TTTAAACAAATGTGCACCAC | −7.7 | −18.8 | 56.3 | −10.4 | −0.2 | −8.8 |
| | SEQ.ID.NO:268 | | | | | | |
| 19 | AGGTGCCAGCGGCGTACTAA | −7.6 | −28.3 | 76.3 | −18.3 | −2.4 | −10.6 |
| | SEQ.ID.NO:269 | | | | | | |
| 440 | TGCAGCATCAAAAGTGTCCA | −7.6 | −23.5 | 67.6 | −15.9 | 0 | −6 |
| | SEQ.ID.NO:270 | | | | | | |
| 485 | AGTCATTCTAATTAAACAAT | −7.6 | −15.3 | 50.5 | −7.7 | 0 | −3.8 |
| | SEQ.ID.NO:271 | | | | | | |
| 537 | TTGCAATATACCACATTTAG | −7.6 | −19 | 57.9 | −11.4 | 0 | −6.6 |
| | SEQ.ID.NO:272 | | | | | | |
| 614 | TTTTTTTTTTTTTGTTTAAA | −7.6 | −14.9 | 50.7 | −7.3 | 0 | −4.6 |
| | SEQ.ID.NO:273 | | | | | | |
| 90 | CGCCGTCGCTTGCAAAGGCG | −7.5 | −29.5 | 74.2 | −18.7 | −3.3 | −11.6 |
| | SEQ.ID.NO:274 | | | | | | |
| 137 | TGCGATCCATTCAACTCGTA | −7.5 | −24 | 67.4 | −15.8 | −0.4 | −4 |
| | SEQ.ID.NO:275 | | | | | | |
| 267 | CATGTACTATCTTGGGGTTG | −7.5 | −22.7 | 68.3 | −15.2 | 0 | −4.8 |
| | SEQ.ID.NO:276 | | | | | | |
| 364 | TTATGTTTTGTGTGAGCCCC | −7.5 | −26.1 | 74.9 | −18.6 | 0 | −3.2 |
| | SEQ.ID.NO:277 | | | | | | |
| 116 | GCTGGAACTGGAAGTCAGAC | −7.4 | −23.1 | 67.8 | −13.8 | −1.9 | −5.8 |
| | SEQ.ID.NO:278 | | | | | | |
| 323 | GGACCTCCAACAACGGCAGT | −7.4 | −27.4 | 73.2 | −20 | 0 | −4.9 |
| | SEQ.ID.NO:279 | | | | | | |
| 344 | ATCACAGAATGGGAACTTTT | −7.4 | −19.6 | 59.4 | −10.6 | −1.6 | −4.9 |
| | SEQ.ID.NO:280 | | | | | | |
| 49 | CAAACGGGTTCGCGCGGCCG | −7.3 | −30.2 | 73.6 | −19.2 | −1.5 | −15.6 |
| | SEQ.ID.NO:281 | | | | | | |
| 189 | TTTTGTAAGCTAGATAACCA | −7.3 | −19.4 | 59.3 | −12.1 | 0 | −5.1 |
| | SEQ.ID.NO:282 | | | | | | |
| 201 | TAACATAAAATCTTTTGTAA | −7.3 | −13.3 | 46.2 | −5.4 | −0.3 | −3.7 |
| | SEQ.ID.NO:283 | | | | | | |
| 228 | TTATCATAGCTTTATTTCGA | −7.3 | −19.2 | 59.8 | −11.9 | 0 | −4.6 |
| | SEQ.ID.NO:284 | | | | | | |
| 261 | CTATCTTGGGGTTGTCTTTC | −7.3 | −24 | 73.7 | −16.7 | 0 | −1.5 |
| | SEQ.ID.NO:285 | | | | | | |
| 486 | TAGTCATTCTAATTAAACAA | −7.3 | −15 | 50 | −7.7 | 0 | −3.8 |
| | SEQ.ID.NO:286 | | | | | | |
| 497 | GACAGAGGTGGTAGTCATTC | −7.3 | −23.4 | 72.2 | −15.4 | −0.4 | −4.7 |
| | SEQ.ID.NO:287 | | | | | | |
| 471 | AACAATCAGGTAACTTCACG | −7.2 | −19.3 | 58 | −11.2 | −0.8 | −4.5 |
| | SEQ.ID.NO:288 | | | | | | |
| 483 | TCATTCTAATTAAACAATCA | −7.2 | −15.2 | 50.1 | −8 | 0 | −3.8 |
| | SEQ.ID.NO:289 | | | | | | |
| 488 | GGTAGTCATTCTAATTAAAC | −7.2 | −17.4 | 55.9 | −10.2 | 0 | −4.4 |
| | SEQ.ID.NO:290 | | | | | | |
| 551 | GTGAAAGCTGCAGTTTGCAA | −7.2 | −22.8 | 66.6 | −13.4 | −2.2 | −9.9 |
| | SEQ.ID.NO:291 | | | | | | |
| 612 | TTTTTTTTTTTGTTTAAACA | −7.2 | −15.6 | 51.9 | −7.4 | 0 | −10 |
| | SEQ.ID.NO:292 | | | | | | |
| 44 | GGGTTCGCGCGGCCGGCGAG | −7.1 | −34.3 | 83.7 | −21.6 | −2.9 | −19.4 |
| | SEQ.ID.NO:293 | | | | | | |
| 221 | AGCTTTATTTCGATGATCTT | −7.1 | −20.6 | 62.9 | −13.5 | 0 | −4.9 |
| | SEQ.ID.NO:294 | | | | | | |
| 425 | GTCCATTTAAGTTTCTTTTT | −7.1 | −20.6 | 64.2 | −13.5 | 0 | −2.7 |
| | SEQ.ID.NO:295 | | | | | | |
| 548 | AAAGCTGCAGTTTGCAATAT | −7.1 | −20.7 | 61.7 | −11.6 | −2 | −9.9 |
| | SEQ.ID.NO:296 | | | | | | |
| 552 | TGTGAAAGCTGCAGTTTGCA | −7.1 | −23.5 | 68.7 | −14.1 | −2.3 | −9.6 |
| | SEQ.ID.NO:297 | | | | | | |
| 138 | CTGCGATCCATTCAACTCGT | −7 | −25.2 | 69.8 | −17.5 | −0.4 | 4.1 |
| | SEQ.ID.NO:298 | | | | | | |
| 333 | GGAACTTTTTGGACCTCCAA | −7 | −23.9 | 67.5 | −15.6 | −1.2 | −8.3 |
| | SEQ.ID.NO:299 | | | | | | |
| 478 | CTAATTAAACAATCAGGTAA | −7 | −15 | 49.3 | −8 | 0 | −3.8 |
| | SEQ.ID.NO:300 | | | | | | |
| 18 | GGTGCCAGCGGCGTACTAAA | −6.9 | −27.6 | 73.7 | −18.3 | −2.4 | −10.6 |
| | SEQ.ID.NO:301 | | | | | | |
| 88 | CCGTCGCTTGCAAAGGCGTG | −6.9 | −28.1 | 73.4 | −17.6 | −3.6 | −12.2 |
| | SEQ.ID.NO:302 | | | | | | |
| 363 | TATGTTTTGTGTGAGCCCCA | −6.9 | −26.7 | 75.6 | −19.8 | 0 | −3.2 |
| | SEQ.ID.NO:303 | | | | | | |
| 395 | CAGAGGGCCCACATTGTCTC | −6.9 | −28.1 | 79.2 | −19.6 | 0 | −11.3 |
| | SEQ.ID.NO:304 | | | | | | |
| 476 | AATTAAACAATCAGGTAACT | −6.9 | −15.5 | 50.3 | −7.7 | −0.7 | −4.3 |
| | SEQ.ID.NO:305 | | | | | | |
| 36 | GCGGCCGGCGAGAGTAAAGG | −6.7 | −28.1 | 73.2 | −19.5 | −0.8 | −12 |
| | SEQ.ID.NO:306 | | | | | | |
| 74 | GGCGTGTGCACTAGGATACC | −6.7 | −27.2 | 76.1 | −18.8 | −1.7 | −8.7 |
| | SEQ.ID.NO:307 | | | | | | |
| 105 | AAGTCAGACTCATGGCGCCG | −6.7 | −26.8 | 73.1 | −18.2 | −0.3 | −12 |
| | SEQ.ID.NO:308 | | | | | | |
| 295 | TTATCTCCCAAATCCTCCAT | −6.7 | −25.2 | 70 | −18.5 | 0 | −1.1 |
| | SEQ.ID.NO:309 | | | | | | |
| 415 | GTTTCTTTTTTCTTGATGAT | −6.7 | −19.8 | 62.8 | −13.1 | 0 | −2.2 |
| | SEQ.ID.NO:310 | | | | | | |
| 422 | CATTTAAGTTTCTTTTTTCT | −6.7 | −18.4 | 59.2 | −11.7 | 0 | −2.6 |
| | SEQ.ID.NO:311 | | | | | | |
| 146 | GGTAACTGCTGCGATCCATT | −6.6 | −25.6 | 71.4 | −19 | 0 | −6.4 |
| | SEQ.ID.NO:312 | | | | | | |
| 317 | CCAACAACGGCAGTACACAG | −6.6 | −23.6 | 65.4 | −17 | 0 | −5.3 |
| | SEQ.ID.NO:313 | | | | | | |
| 345 | CATCACAGAATGGGAACTTT | −6.6 | −20.2 | 60.3 | −12 | −1.6 | −4.9 |
| | SEQ.ID.NO:314 | | | | | | |
| 526 | CACATTTAGAATCCAGCGAA | −6.6 | −21 | 60.8 | −14.4 | 0 | −4.1 |
| | SEQ.ID.NO:315 | | | | | | |
| 604 | TTTGTTTAAACAAATGTGCA | −6.6 | −17.1 | 53.8 | −7.6 | −1.7 | −13.8 |
| | SEQ.ID.NO:316 | | | | | | |
| 80 | TGCAAAGGCGTGTGCACTAG | −6.5 | −24.6 | 69.4 | −16 | −1.8 | −12 |
| | SEQ.ID.NO:317 | | | | | | |
| 106 | GAAGTCAGACTCATGGCGCC | −6.5 | −26.6 | 74.5 | −18.8 | −0.3 | −10.6 |
| | SEQ.ID.NO:318 | | | | | | |
| 147 | TGGTAACTGCTGCGATCCAT | −6.5 | −25.5 | 70.9 | −19 | 0 | −6.4 |
| | SEQ.ID.NO:319 | | | | | | |
| 148 | ATGGTAACTGCTGCGATCCA | −6.5 | −25.5 | 70.9 | −19 | 0 | −6.4 |
| | SEQ.ID.NO:320 | | | | | | |
| 188 | TTTGTAAGCTAGATAACCAA | −6.5 | −18.6 | 57.1 | −12.1 | 0 | −5.1 |
| | SEQ.ID.NO:321 | | | | | | |
| 246 | CTTTCTGGATGTGAAGGTTT | −6.5 | −21.9 | 66.4 | −15.4 | 0 | 3.3 |
| | SEQ.ID.NO:322 | | | | | | |
| 298 | GCTTTATCTCCCAAATCCTC | −6.5 | −25.3 | 71.7 | −18.8 | 0 | −2.8 |
| | SEQ.ID.NO:323 | | | | | | |
| 308 | GCAGTACACAGCTTTATCTC | −6.5 | −23.5 | 71.4 | −17 | 0 | 5.4 |
| | SEQ.ID.NO:324 | | | | | | |
| 91 | GCGCCGTCGCTTGCAAAGGC | −6.4 | −30.5 | 78.2 | −21.2 | −2.9 | −12.2 |
| | SEQ.ID.NO:325 | | | | | | |
| 187 | TTGTAAGCTAGATAACCAAT | −6.4 | −18.5 | 56.7 | −12.1 | 0 | −4.6 |
| | SEQ.ID.NO:326 | | | | | | |
| 233 | AAGGTTTATCATAGCTTTAT | −6.3 | −19 | 60 | −12.7 | 0 | −4.6 |
| | SEQ.ID.NO:327 | | | | | | |
| 243 | TCTGGATGTGAAGGTTTATC | −6.3 | −20.9 | 64.6 | −14.6 | 0 | −2.5 |
| | SEQ.ID.NO:328 | | | | | | |
| 249 | TGTCTTTCTGGATGTGAAGG | −6.3 | −22.1 | 67.1 | −15.8 | 0 | −3.4 |
| | SEQ.ID.NO:329 | | | | | | |
| 294 | TATCTCCCAAATCCTCCATG | −6.3 | −25.1 | 69.5 | −18.8 | 0 | −3.9 |
| | SEQ.ID.NO:330 | | | | | | |
| 157 | CCAGCAGCAATGGTAACTGC | −6.1 | −25.3 | 71 | −16.7 | −2.5 | −7.6 |
| | SEQ.ID.NO:331 | | | | | | |
| 245 | TTTCTGGATGTGAAGGTTTA | −6.1 | −20.7 | 63.8 | −14.6 | 0 | −3.3 |
| | SEQ.ID.NO:332 | | | | | | |
| 391 | GGGCCCACATTGTCTCCAGT | −6.1 | −30.7 | 84.7 | −23.3 | 0 | −10.6 |
| | SEQ.ID.NO:333 | | | | | | |
| 437 | AGCATCAAAAGTGTCCATTT | −6.1 | −21.2 | 63.1 | −15.1 | 0 | −4.1 |
| | SEQ.ID.NO:334 | | | | | | |
| 445 | TGATTTGCAGCATCAAAAGT | −6.1 | −20 | 60.2 | −13 | −0.7 | −7 |
| | SEQ.ID.NO:335 | | | | | | |
| 553 | ATGTGAAAGCTGCAGTTTGC | −6.1 | −22.8 | 67.5 | −15.3 | −1.2 | −9.9 |
| | SEQ.ID.NO:336 | | | | | | |
| 244 | TTCTGGATGTGAAGGTTTAT | −6 | −20.6 | 63.4 | −14.6 | 0 | −3 |
| | SEQ.ID.NO:337 | | | | | | |
| 310 | CGGCAGTACACAGCTTTATC | −6 | −24.2 | 70.4 | −17.4 | −0.6 | −5.5 |
| | SEQ.ID.NO:338 | | | | | | |
| 367 | TCGTTATGTTTTGTGTGAGC | −6 | −22.5 | 68.6 | −16.5 | 0 | −3 |
| | SEQ.ID.NO:339 | | | | | | |
| 470 | ACAATCAGGTAACTTCACGA | −6 | −20.6 | 61.1 | −13.7 | −0.8 | −5 |
| | SEQ.ID.NO:340 | | | | | | |
| 550 | TGAAAGCTGCAGTTTGCAAT | −6 | −21.6 | 63.4 | −13.4 | −2.2 | −9.9 |
| | SEQ.ID.NO:341 | | | | | | |
| 584 | CCACGATGTTTCAACAAGAC | −6 | −21.1 | 61.1 | −14.6 | −0.2 | −5.9 |
| | SEQ.ID.NO:342 | | | | | | |
| 20 | AAGGTGCCAGCGGCGTACTA | −5.9 | −28.3 | 76.3 | −20.7 | −1.7 | −9.9 |
| | SEQ.ID.NO:343 | | | | | | |
| 75 | AGGCGTGTGCACTAGGATAC | −5.9 | −25.2 | 72.8 | −17.6 | −1.7 | −9.4 |
| | SEQ.ID.NO:344 | | | | | | |
| 443 | ATTTGCAGCATCAAAAGTGT | −5.9 | −20.6 | 61.9 | −13.8 | −0.7 | −7.5 |
| | SEQ.ID.NO:345 | | | | | | |
| 601 | GTTTAAACAAATGTGCACCA | −5.9 | −19.8 | 58.5 | −13.2 | 0 | −9.1 |
| | SEQ.ID.NO:346 | | | | | | |
| 272 | AAAAGCATGTACTATCTTGG | −5.8 | −18.7 | 57.7 | −12.9 | 0 | −4.9 |
| | SEQ.ID.NO:347 | | | | | | |
| 332 | GAACTTTTTGGACCTCCAAC | −5.8 | −22.9 | 65.6 | −15.6 | −1.4 | −8.5 |
| | SEQ.ID.NO:348 | | | | | | |
| 580 | GATGTTTCAACAAGACAAAT | −5.8 | −16.7 | 52.8 | −10.4 | −0.2 | −5.9 |
| | SEQ.ID.NO:349 | | | | | | |
| 582 | ACGATGTTTCAACAAGACAA | −5.8 | −18.4 | 55.8 | −12.1 | −0.2 | −5.9 |
| | SEQ.ID.NO:350 | | | | | | |
| 605 | TTTTGTTTAAACAAATGTGC | −5.8 | −16.5 | 52.8 | −7.6 | −2 | −14.3 |
| | SEQ.ID.NO:351 | | | | | | |
| 26 | AGAGTAAAGGTGCCAGCGGC | −5.7 | −26.6 | 74.1 | −19.5 | −1.3 | −9.2 |
| | SEQ.ID.NO:352 | | | | | | |
| 104 | AGTCAGACTCATGGCGCCGT | −5.7 | −28.7 | 78.8 | −21.1 | −0.3 | −12 |
| | SEQ.ID.NO:353 | | | | | | |
| 113 | GGAACTGGAAGTCAGACTCA | −5.7 | −22.4 | 66.4 | −14.9 | −1.8 | −7.3 |
| | SEQ.ID.NO:354 | | | | | | |
| 300 | CAGCTTTATCTCCCAAATCC | −5.7 | −24.7 | 69.7 | −19 | 0 | −4.5 |
| | SEQ.ID.NO:355 | | | | | | |
| 462 | GTAACTTCACGACAAGCTGA | −5.7 | −21.6 | 63.1 | −15.9 | 0 | −5.1 |
| | SEQ.ID.NO:356 | | | | | | |
| 22 | TAAAGGTGCCAGCGGCGTAC | −5.6 | −26.7 | 72.2 | −18.7 | −2.4 | −10.6 |
| | SEQ.ID.NO:357 | | | | | | |
| 83 | GCTTGCAAAGGCGTGTGCAC | −5.6 | −26.8 | 74.2 | −18.9 | −2.3 | −11.6 |
| | SEQ.ID.NO:358 | | | | | | |
| 396 | TCAGAGGGCCCACATTGTCT | −5.6 | −28.1 | 79.2 | −21.2 | 0 | −10.5 |
| | SEQ.ID.NO:359 | | | | | | |
| 410 | TTTTTTCTTGATGATCAGAG | −5.6 | −18.8 | 59.8 | −13.2 | 0 | −6.8 |
| | SEQ.ID.NO:360 | | | | | | |
| 474 | TTAAACAATCAGGTAACTTC | −5.6 | −16.7 | 53.5 | −10.2 | −0.8 | −3.7 |
| | SEQ.ID.NO:361 | | | | | | |
| 549 | GAAAGCTGCAGTTTGCAATA | −5.6 | −21.3 | 63 | −13.5 | −2.2 | −9.9 |
| | SEQ.ID.NO:362 | | | | | | |
| 581 | CGATGTTTCAACAAGACAAA | −5.6 | −17.5 | 53.6 | −11.4 | −0.2 | −5.9 |
| | SEQ.ID.NO:363 | | | | | | |
| 1 | AAAGCACCGACTCCGCGATC | −5.5 | −26.6 | 69.5 | −20.3 | −0.6 | −7.2 |
| | SEQ.ID.NO:364 | | | | | | |
| 21 | AAAGGTGCCAGCGGCGTACT | −5.5 | −27.9 | 74.5 | −20 | −2.4 | −10.6 |
| | SEQ.ID.NO:365 | | | | | | |
| 362 | ATGTTTTGTGTGAGCCCCAT | −5.5 | −27 | 76.1 | −21.5 | 0 | −3.2 |
| | SEQ.ID.NO:366 | | | | | | |
| 417 | AAGTTTCTTTTTTCTTGATG | −5.5 | −18.5 | 59.4 | −13 | 0 | −2.4 |
| | SEQ.ID.NO:367 | | | | | | |
| 489 | TGGTAGTCATTCTAATTAAA | −5.5 | −17.2 | 55.3 | −11.7 | 0 | −3.8 |
| | SEQ.ID.NO:368 | | | | | | |
| 611 | TTTTTTTTTTGTTTAAACAA | −5.5 | −14.8 | 49.8 | −7.4 | −0.8 | −12 |
| | SEQ.ID.NO:369 | | | | | | |
| 115 | CTGGAACTGGAAGTCAGACT | −5.4 | −22.2 | 65.5 | −15.3 | −1.4 | −7.1 |
| | SEQ.ID.NO:370 | | | | | | |
| 150 | CAATGGTAACTGCTGCGATC | −5.4 | −22.8 | 65.3 | −17.4 | 0 | −6.4 |
| | SEQ.ID.NO:371 | | | | | | |
| 197 | ATAAAATCTTTTGTAAGCTA | −5.4 | −15.8 | 51.7 | −10.4 | 0 | −5.1 |
| | SEQ.ID.NO:372 | | | | | | |
| 112 | GAACTGGAAGTCAGACTCAT | −5.3 | −21.2 | 63.8 | −14.4 | −1.4 | −7.3 |
| | SEQ.ID.NO:373 | | | | | | |
| 226 | ATCATAGCTTTATTTCGATG | −5.3 | −19.4 | 59.9 | −14.1 | 0 | −4.7 |
| | SEQ.ID.NO:374 | | | | | | |
| 229 | TTTATCATAGCTTTATTTCG | −5.3 | −18.7 | 58.8 | −13.4 | 0 | −4.6 |
| | SEQ.ID.NO:375 | | | | | | |
| 101 | CAGACTCATGGCGCCGTCGC | −5.2 | −30.1 | 78.9 | −22.4 | −2.5 | −12.1 |
| | SEQ.ID.NO:376 | | | | | | |
| 190 | CTTTTGTAAGCTAGATAACC | −5.2 | −19.6 | 60 | −14.4 | 0 | −5.1 |
| | SEQ.ID.NO:377 | | | | | | |
| 223 | ATAGCTTTATTTCGATGATC | −5.2 | −19.3 | 60 | −14.1 | 0 | −4.7 |
| | SEQ.ID.NO:378 | | | | | | |
| 346 | CCATCACAGAATGGGAACTT | −5.2 | −22.1 | 63.6 | −15.3 | −1.6 | −5.3 |
| | SEQ.ID.NO:379 | | | | | | |
| 222 | TAGCTTTATTTCGATGATCT | −5.1 | −20.2 | 62 | −15.1 | 0 | 4.9 |
| | SEQ.ID.MO:380 | | | | | | |
| 234 | GAAGGTTTATCATAGCTTTA | −5.1 | −19.6 | 61.4 | −14.5 | 0 | −4.6 |
| | SEQ.ID.140:381 | | | | | | |
| 307 | CAGTACACAGCTTTATCTCC | −5.1 | −23.7 | 70.7 | −18.6 | 0 | −5.3 |
| | SEQ.ID.NO:382 | | | | | | |
| 312 | AACGGCAGTACACAGCTTTA | −5.1 | −23.3 | 67.2 | −17.4 | −0.6 | −5.5 |
| | SEQ.ID.NO:383 | | | | | | |
| 427 | GTGTCCATTTAAGTTTCTTT | −5.1 | −21.6 | 66.7 | −16.5 | 0 | −2.7 |
| | SEQ.ID.NO:384 | | | | | | |
| 76 | AAGGCGTGTGCACTAGGATA | −5 | −24.3 | 69.8 | −17.6 | −1.7 | −9.4 |
| | SEQ.ID.NO:385 | | | | | | |
| 158 | CCCAGCAGCAATGGTAACTG | −5 | −25.5 | 70.4 | −19.6 | −0.8 | −6 |
| | SEQ.ID.IJO:386 | | | | | | |
| 186 | TGTAAGCTAGATAACCAATT | −4.9 | −18.5 | 56.7 | −13.6 | 0 | −5.1 |
| | SEQ.ID.NO:387 | | | | | | |
| 200 | AACATAAAATCTTTTGTAAG | −4.9 | −13.6 | 46.8 | −8.1 | −0.3 | −3.5 |
| | SEQ.ID.NO:388 | | | | | | |
| 484 | GTCATTCTAATTAAACAATC | −4.9 | −15.7 | 51.5 | −10.8 | 0 | −3.8 |
| | SEQ.ID.NO:389 | | | | | | |
| 436 | GCATCAAAAGTGTCCATTTA | −4.8 | −20.9 | 62.3 | −16.1 | 0 | −3.4 |
| | SEQ.ID.NO:390 | | | | | | |
| 359 | TTTTGTGTGAGCCCCATCAC | −4.7 | −27.1 | 76.3 | −21 | −1.3 | −5.6 |
| | SEQ.ID.NO:391 | | | | | | |
| 469 | CAATCAGGTAACTTCACGAC | −4.7 | −20.6 | 61.1 | −15.1 | −0.6 | −4.8 |
| | SEQ.ID.NO:392 | | | | | | |
| 473 | TAAACAATCAGGTAACTTCA | −4.7 | −17.3 | 54.4 | −11.7 | −0.8 | −3.7 |
| | SEQ.ID.NO:393 | | | | | | |
| 108 | TGGAAGTCAGACTCATGGCG | −4.6 | −24 | 69.1 | −19.4 | 0 | −7.3 |
| | SEQ.ID.NO:394 | | | | | | |
| 199 | ACATAAAATCTTTTGTAAGC | −4.6 | −16.1 | 52.2 | −11.5 | 0 | −4.5 |
| | SEQ.ID.NO:395 | | | | | | |
| 542 | GCAGTTTGCAATATACCACA | −4.6 | −22.9 | 66.3 | −16.8 | −1.4 | −6.8 |
| | SEQ.ID.NO:396 | | | | | | |
| 114 | TGGAACTGGAAGTCAGACTC | −4.5 | −21.7 | 65.1 | −16.1 | −1 | −7.3 |
| | SEQ.ID.NO:397 | | | | | | |
| 456 | TCACGACAAGCTGATTTGCA | −4.5 | −22.9 | 65.6 | −17.5 | −0.8 | −5.1 |
| | SEQ.ID.NO:398 | | | | | | |
| 461 | TAACTTCACGACAAGCTGAT | −4.5 | −20.4 | 60.2 | −15.9 | 0 | −5.1 |
| | SEQ.ID.NO:399 | | | | | | |
| 613 | TTTTTTTTTTTTGTTTAAAC | −4.5 | −15 | 50.9 | −10 | 0 | −7.8 |
| | SEQ.ID.NO:400 | | | | | | |
| 303 | ACACAGCTTTATCTCCCAAA | −4.4 | −23.4 | 66.9 | −19 | 0 | −4.5 |
| | SEQ.ID.NO:401 | | | | | | |
| 247 | TCTTTCTGGATGTGAAGGTT | −4.3 | −22.2 | 67.7 | −17.9 | 0 | −3.3 |
| | SEQ.ID.NO:402 | | | | | | |
| 306 | AGTACACAGCTTTATCTCCC | −4.3 | −25 | 73.4 | −20.7 | 0 | −5.3 |
| | SEQ.ID.NO:403 | | | | | | |
| 366 | CGTTATGTTTTGTGTGAGCC | −4.3 | −24.1 | 70.8 | −19.8 | 0 | −3.2 |
| | SEQ.ID.NO:404 | | | | | | |
| 156 | CAGCAGCAATGGTAACTGCT | −4.2 | −24.2 | 69.3 | −16.7 | −3.3 | −9 |
| | SEQ.ID.NO:405 | | | | | | |
| 302 | CACAGCTTTATCTCCCAAAT | −4.2 | −23.2 | 66.3 | −19 | 0 | −4.5 |
| | SEQ.ID.NO:406 | | | | | | |
| 463 | GGTAACTTCACGACAAGCTG | −4.2 | −22.2 | 64.3 | −18 | 0 | −5.1 |
| | SEQ.ID.NO:407 | | | | | | |
| 583 | CACGATGTTTCAACAAGACA | −4.2 | −19.8 | 58.8 | −15.6 | 0.1 | −5.4 |
| | SEQ.ID.NO:408 | | | | | | |
| 607 | TTTTTTGTTTAAACAAATGT | −4.2 | −14.9 | 49.7 | −7.6 | −2 | −14.3 |
| | SEQ.ID.NO:409 | | | | | | |
| 48 | AAACGGGTTCGCGCGGCCGG | −4.1 | −30.7 | 74.8 | −22.6 | −1.9 | −16.1 |
| | SEQ.ID.NO:410 | | | | | | |
| 299 | AGCTTTATCTCCCAAATCCT | −4.1 | −24.9 | 70.4 | −20.8 | 0 | −4.3 |
| | SEQ.ID.NO:411 | | | | | | |
| 401 | GATGATCAGAGGGCCCACAT | −4.1 | −26.7 | 74.3 | −21 | 0 | −11.3 |
| | SEQ.ID.NO:412 | | | | | | |
| 402 | TGATGATCAGAGGGCCCACA | −4.1 | −26.7 | 74.1 | −21 | 0 | −11.3 |
| | SEQ.ID.NO:413 | | | | | | |
| 606 | TTTTTGTTTAAACAAATGTG | −4.1 | −14.8 | 49.4 | −7.6 | −2 | −14.3 |
| | SEQ.ID.NO:414 | | | | | | |
| 77 | AAAGGCGTGTGCACTAGGAT | −4 | −23.9 | 68.1 | −18.2 | −1.7 | −9.4 |
| | SEQ.ID.NO:415 | | | | | | |
| 109 | CTGGAAGTCAGACTCATGGC | −4 | −24.1 | 71 | −19.4 | −0.5 | −6.9 |
| | SEQ.ID.NO:416 | | | | | | |
| 311 | ACGGCAGTACACAGCTTTAT | −4 | −24 | 69.4 | −19.2 | −0.6 | −5.5 |
| | SEQ.ID.NO:417 | | | | | | |
| 544 | CTGCAGTTTGCAATATACCA | −4 | −22.9 | 66.4 | −16.7 | −2.2 | −7.4 |
| | SEQ.ID.NO:418 | | | | | | |
| 139 | GCTGCGATCCATTCAACTCG | −3.9 | −25.8 | 70.6 | −21.4 | −0.1 | −5.8 |
| | SEQ.ID.NO:419 | | | | | | |
| 174 | AACCAATTGCAGCTGTCCCA | −3.9 | −27.1 | 73.5 | −22.5 | 0 | −8.7 |
| | SEQ.ID.NO:420 | | | | | | |
| 301 | ACAGCTTTATCTCCCAAATC | −3.9 | −22.9 | 66.6 | −19 | 0 | 4.5 |
| | SEQ.ID.NO:421 | | | | | | |
| 328 | TTTTTGGACCTCCAACAACG | −3.9 | −22.9 | 64 | −17.5 | −1.4 | −8.5 |
| | SEQ.ID.NO:422 | | | | | | |
| 331 | AACTTTTTGGACCTCCAACA | −3.9 | −23 | 65.5 | −17.6 | 1.4 | −8.5 |
| | SEQ.ID.NO:423 | | | | | | |
| 154 | GCAGCAATGGTAACTGCTGC | −3.8 | −25.3 | 72 | −16.7 | −4.8 | −12 |
| | SEQ.ID.NO:424 | | | | | | |
| 163 | GCTGTCCCAGCAGCAATGGT | −3.8 | −29.7 | 82 | −22.4 | −3.5 | −9.7 |
| | SEQ.ID.NO:425 | | | | | | |
| 305 | GTACACAGCTTTATCTCCCA | −3.8 | −25.7 | 74.2 | −21.9 | 0 | −4.6 |
| | SEQ.ID.NO:426 | | | | | | |
| 339 | AGAATGGGAACTTTTTGGAC | −3.8 | −19.7 | 59.7 | −15.4 | −0.2 | −2.8 |
| | SEQ.ID.NO:427 | | | | | | |
| 397 | ATCAGAGGGCCCACATTGTC | −3.8 | −27.2 | 77.2 | −21.8 | 0 | −11.3 |
| | SEQ.ID.NO:428 | | | | | | |
| 545 | GCTGCAGTTTGCAATATACC | −3.8 | −24 | 69.4 | −18 | −2.2 | −8.7 |
| | SEQ.ID.NO:429 | | | | | | |
| 2 | TAAAGCACCGACTCCGCGAT | −3.7 | −25.9 | 67.7 | −21.4 | −0.6 | −7.2 |
| | SEQ.ID.NO:430 | | | | | | |
| 98 | ACTCATGGCGCCGTCGCTTG | −3.7 | −29.8 | 78.4 | −22.8 | −3.3 | −12 |
| | SEQ.ID.NO:431 | | | | | | |
| 145 | GTAACTGCTGCGATCCATTC | −3.7 | −24.8 | 70.5 | −21.1 | 0 | −6.4 |
| | SEQ.ID.NO:432 | | | | | | |
| 178 | AGATAACCAATTGCAGCTGT | −3.7 | −22.3 | 64.9 | −17.7 | 0 | −9.7 |
| | SEQ.ID.NO:433 | | | | | | |
| 457 | TTCACGACAAGCTGATTTGC | −3.7 | −22.3 | 64.8 | −18.6 | 0 | −5.1 |
| | SEQ.ID.NO:434 | | | | | | |
| 6 | GTACTAAAGCACCGACTCCG | −3.6 | −24.7 | 67.2 | −21.1 | 0 | −4.1 |
| | SEQ.ID.NO:435 | | | | | | |
| 78 | CAAAGGCGTGTGCACTAGGA | −3.6 | −24.6 | 69.3 | −19.9 | −0.9 | −9.4 |
| | SEQ.ID.NO:436 | | | | | | |
| 82 | CTTGCAAAGGCGTGTGCACT | −3.6 | −25.9 | 72 | −20.3 | −2 | −11.1 |
| | SEQ.ID.NO:437 | | | | | | |
| 175 | TAACCAATTGCAGCTGTCCC | −3.6 | −26.1 | 71.9 | −21.7 | 0 | −9.4 |
| | SEQ.ID.NO:438 | | | | | | |
| 428 | AGTGTCCATTTAAGTTTCTT | −3.6 | −21.5 | 66.6 | −17.9 | 0 | −2.6 |
| | SEQ.ID.NO:439 | | | | | | |
| 155 | AGCAGCAATGGTAACTGCTG | −3.5 | −23.5 | 68 | −16.7 | −3.3 | −9 |
| | SEQ.ID.NO:440 | | | | | | |
| 430 | AAAGTGTCCATTTAAGTTTC | −3.5 | −19.1 | 59.7 | −15.6 | 0 | −2.6 |
| | SEQ.ID.NO:441 | | | | | | |
| 444 | GATTTGCAGCATCAAAAGTG | −3.5 | −20 | 60.2 | −15.6 | −0.7 | −6.8 |
| | SEQ.ID.NO:442 | | | | | | |
| 468 | AATCAGGTAACTTCACGACA | −3.5 | −20.6 | 61.1 | −16.2 | −0.8 | −5 |
| | SEQ.ID.NO:443 | | | | | | |
| 477 | TAATTAAACAATCAGGTAAC | −3.5 | −14.3 | 48 | −10.8 | 0 | −3.5 |
| | SEQ.ID.NO:444 | | | | | | |
| 253 | GGGTTGTCTTTCTGGATGTG | −3.4 | −24.7 | 74.7 | −21.3 | 0 | −3 |
| | SEQ.ID.NO:445 | | | | | | |
| 409 | TTTTTCTTGATGATCAGAGG | −3.4 | −19.9 | 62.2 | −16.5 | 0 | −6.8 |
| | SEQ.ID.NO:446 | | | | | | |
| 442 | TTTGCAGCATCAAAAGTGTC | −3.4 | −21 | 63.4 | −17.1 | −0.2 | −7.5 |
| | SEQ.ID.NO:447 | | | | | | |
| 198 | CATAAAATCTTTTGTAAGCT | −3.3 | −16.8 | 53.5 | −13.5 | 0 | −4.8 |
| | SEQ.ID.NO:448 | | | | | | |
| 235 | TGAAGGTTTATCATAGCTTT | −3.3 | −19.9 | 61.9 | −16.6 | 0 | −5.8 |
| | SEQ.ID.NO:449 | | | | | | |
| 185 | GTAAGCTAGATAACCAATTG | −3.2 | −18.5 | 56.7 | −15.3 | 0 | −5.9 |
| | SEQ.ID.NO:450 | | | | | | |
| 336 | ATGGGAACTTTTTGGACCTC | −3.2 | −23.1 | 67.3 | −19.4 | −0.2 | −3.5 |
| | SEQ.ID.NO:451 | | | | | | |
| 424 | TCCATTTAAGTTTCTTTTTT | −3.2 | −19.5 | 61.2 | −16.3 | 0 | −2.7 |
| | SEQ.ID.NO:452 | | | | | | |
| 438 | CAGCATCAAAAGTGTCCATT | −3.2 | −21.8 | 63.9 | −18.6 | 0 | −4.1 |
| | SEQ.ID.NO:453 | | | | | | |
| 92 | GGCGCCGTCGCTTGCAAAGG | −3.1 | −29.9 | 76.7 | −23.5 | −3.3 | −13.3 |
| | SEQ.ID.NO:454 | | | | | | |
| 107 | GGAAGTCAGACTCATGGCGC | −3.1 | −25.8 | 73.5 | −22.1 | −0.3 | −7.3 |
| | SEQ.ID.NO:455 | | | | | | |
| 554 | AATGTGAAAGCTGCAGTTTG | −3.1 | −20.3 | 61.1 | −16.2 | −0.2 | −9.9 |
| | SEQ.ID.NO:456 | | | | | | |
| 608 | TTTTTTTGTTTAAACAAATG | −3.1 | −13.8 | 47.3 | −7.6 | −2 | −14.3 |
| | SEQ.ID.NO:457 | | | | | | |
| 10 | CGGCGTACTAAAGCACCGAC | −3 | −25.2 | 67 | −21.2 | −0.9 | −5.5 |
| | SEQ.ID.NO:458 | | | | | | |
| 327 | TTTTGGACCTCCAACAACGG | −3 | −24 | 66 | −19.5 | −1.4 | −8.5 |
| | SEQ.ID.NO:459 | | | | | | |
| 361 | TGTTTTGTGTGAGCCCCATC | −3 | −27.4 | 78 | −24.4 | 0 | −3.2 |
| | SEQ.ID.NO:460 | | | | | | |
| 567 | GACAAATGCCATAAATGTGA | −3 | −18.7 | 55.9 | −15.7 | 0 | −3.4 |
| | SEQ.ID.NO:461 | | | | | | |
| 7 | CGTACTAAAGCACCGACTCC | −2.9 | −24.7 | 67.2 | −21.8 | 0 | −4.3 |
| | SEQ.ID.NO:462 | | | | | | |
| 111 | AACTGGAAGTCAGACTCATG | −2.9 | −20.6 | 62.4 | −16.2 | −1.4 | −7.3 |
| | SEQ.ID.NO:463 | | | | | | |
| 225 | TCATAGCTTTATTTCGATGA | −2.9 | −20 | 61.2 | −17.1 | 0 | −4.7 |
| | SEQ.ID.NO:464 | | | | | | |
| 343 | TCACAGAATGGGAACTTTTT | −2.9 | −19.7 | 59.8 | −15.6 | −1.1 | −4.9 |
| | SEQ.ID.NO:465 | | | | | | |
| 149 | AATGGTAACTGCTGCGATCC | −2.8 | −24.1 | 67.7 | −21.3 | 0 | −6.4 |
| | SEQ.ID.NO:466 | | | | | | |
| 326 | TTTGGACCTCCAACAACGGC | −2.8 | −25.7 | 69.4 | −21.4 | −1.4 | −8.5 |
| | SEQ.ID.NO:467 | | | | | | |
| 543 | TGCAGTTTGCAATATACCAC | −2.8 | −22.2 | 65.1 | −17.3 | −2.1 | −7.2 |
| | SEQ.ID.NO:468 | | | | | | |
| 241 | TGGATGTGAAGGTTTATCAT | −2.7 | −20.3 | 62.3 | −17.1 | −0.2 | −5.4 |
| | SEQ.ID.NO:469 | | | | | | |
| 266 | ATGTACTATCTTGGGGTTGT | −2.7 | −23.2 | 70.6 | −20.5 | 0 | −4.8 |
| | SEQ.ID.NO:470 | | | | | | |
| 416 | AGTTTCTTTTTTCTTGATGA | −2.7 | −19.8 | 63 | −17.1 | 0 | −2.2 |
| | SEQ.ID.NO:471 | | | | | | |
| 256 | TTGGGGTTGTCTTTCTGGAT | −2.6 | −24.8 | 74.4 | −22.2 | 0 | −3 |
| | SEQ.ID.NO:472 | | | | | | |
| 365 | GTTATGTTTTGTGTGAGCCC | −2.6 | −25.3 | 74.7 | −22.7 | 0 | −3.2 |
| | SEQ.ID.NO:473 | | | | | | |
| 452 | GACAAGCTGATTTGCAGCAT | −2.6 | −23.3 | 67.7 | −17.3 | −3.4 | −8.6 |
| | SEQ.ID.NO:474 | | | | | | |
| 610 | TTTTTTTTTGTTTAAACAAA | −2.6 | −14 | 47.9 | −8.4 | −1.8 | −14 |
| | SEQ.ID.NO:475 | | | | | | |
| 97 | CTCATGGCGCCGTCGCTTGC | −2.5 | −31.4 | 81.9 | −25.6 | −3.3 | −11.3 |
| | SEQ.ID.NO:476 | | | | | | |
| 254 | GGGGTTGTCTTTCTGGATGT | −2.5 | −25.9 | 77.8 | −23.4 | 0 | −3 |
| | SEQ.ID.NO:477 | | | | | | |
| 96 | TCATGGCGCCGTCGCTTGCA | −2.4 | −31.2 | 81 | −25.6 | −2.5 | −14.4 |
| | SEQ.ID.NO:478 | | | | | | |
| 176 | ATAACCAATTGCAGCTGTCC | −2.4 | −24.1 | 68.4 | −20.9 | 0 | −9.4 |
| | SEQ.ID.NO:479 | | | | | | |
| 441 | TTGCAGCATCAAAAGTGTCC | −2.4 | −22.9 | 66.8 | −20 | 0 | −7.5 |
| | SEQ.ID.NO:480 | | | | | | |
| 446 | CTGATTTGCAGCATCAAAAG | −2.4 | −19.7 | 59.1 | −16.4 | −0.7 | −7.2 |
| | SEQ.ID.NO:481 | | | | | | |
| 458 | CTTCACGACAAGCTGATTTG | −2.4 | −21.4 | 62.7 | −19 | 0 | −5.1 |
| | SEQ.ID.NO:482 | | | | | | |
| 224 | CATAGCTTTATTTCGATGAT | −2.3 | −19.6 | 59.8 | −17.3 | 0 | −4.7 |
| | SEQ.ID.NO:483 | | | | | | |
| 230 | GTTTATCATAGCTTTATTTC | −2.3 | −19.1 | 61.4 | −16.8 | 0 | −4.6 |
| | SEQ.ID.NO:484 | | | | | | |
| 242 | CTGGATGTGAAGGTTTATCA | −2.3 | −21.2 | 64.3 | −18.4 | −0.2 | 5.4 |
| | SEQ.ID.NO:485 | | | | | | |
| 360 | GTTTTGTGTGAGCCCCATCA | −2.3 | −28.1 | 79.2 | −25.8 | 0 | −3.2 |
| | SEQ.ID.NO:486 | | | | | | |
| 429 | AAGTGTCCATTTAAGTTTCT | −2.3 | −20.7 | 63.9 | −18.4 | 0 | −2.6 |
| | SEQ.ID.NO:487 | | | | | | |
| 265 | TGTACTATCTTGGGGTTGTC | −2.2 | −23.6 | 72.4 | −21.4 | 0 | −4.8 |
| | SEQ.ID.NO:488 | | | | | | |
| 400 | ATGATCAGAGGGCCCACATT | −2.2 | −26.2 | 73.3 | −22.4 | 0 | −11.3 |
| | SEQ.ID.NO:489 | | | | | | |
| 9 | GGCGTACTAAAGCACCGACT | −2.1 | −25.3 | 68.6 | −22.2 | −0.9 | −4.4 |
| | SEQ.ID.NO:490 | | | | | | |
| 196 | TAAAATCTTTTGTAAGCTAG | −2.1 | −15.8 | 51.8 | −13.7 | 0 | −5.1 |
| | SEQ.ID.NO:491 | | | | | | |
| 439 | GCAGCATCAAAAGTGTCCAT | −2.1 | −23.5 | 67.7 | −21.4 | 0 | −4.7 |
| | SEQ.ID.NO:492 | | | | | | |
| 455 | CACGACAAGCTGATTTGCAG | −2.1 | −22.5 | 64.5 | −19.5 | −0.8 | −5.2 |
| | SEQ.ID.NO:493 | | | | | | |
| 459 | ACTTCACGACAAGCTGATTT | −2.1 | −21.6 | 63.3 | −19.5 | 0 | −5.1 |
| | SEQ.ID.NO:494 | | | | | | |
| 450 | CAAGCTGATTTGCAGCATCA | −2 | −23.6 | 68.5 | −17.4 | −4.2 | −9 |
| | SEQ.ID.NO:495 | | | | | | |
| 153 | CAGCAATGGTAACTGCTGCG | −1.9 | −24.3 | 67.9 | −19.6 | −2.8 | −10.8 |
| | SEQ.ID.NO:496 | | | | | | |
| 169 | ATTGCAGCTGTCCCAGCAGC | −1.9 | −29.9 | 83.8 | −24.4 | −3.6 | −11.3 |
| | SEQ.ID.NO:497 | | | | | | |
| 240 | GGATGTGAAGGTTTATCATA | −1.9 | −20 | 61.8 | −17.6 | −0.2 | −5.4 |
| | SEQ.ID.NO:498 | | | | | | |
| 423 | CCATTTAAGTTTCTTTTTTC | −1.9 | −19.5 | 61.2 | −17.6 | 0 | −2.7 |
| | SEQ.ID.NO:499 | | | | | | |
| 609 | TTTTTTTTGTTTAAACAAAT | −1.9 | −13.9 | 47.6 | −9.1 | −1.7 | −14 |
| | SEQ.ID.NO:500 | | | | | | |
| 255 | TGGGGTTGTCTTTCTGGATG | −1.8 | −24.7 | 73.8 | −22.9 | 0 | −3 |
| | SEQ.ID.NO:501 | | | | | | |
| 340 | CAGAATGGGAACTTTTTGGA | −1.8 | −20.2 | 60.4 | −17.9 | −0.2 | −2.9 |
| | SEQ.ID.NO:502 | | | | | | |
| 467 | ATCAGGTAACTTCACGACAA | −1.8 | −20.6 | 61.1 | −17.9 | −0.8 | −5 |
| | SEQ.ID.NO:503 | | | | | | |
| 17 | GTGCCAGCGGCGTACTAAAG | −1.6 | −26.4 | 71.6 | −22.4 | −2.4 | −10.6 |
| | SEQ.ID.NO:504 | | | | | | |
| 84 | CGCTTGCAAAGGCGTGTGCA | −1.6 | −27.4 | 73.5 | −22.8 | −3 | −11.2 |
| | SEQ.ID.NO:505 | | | | | | |
| 358 | TTTGTGTGAGCCCCATCACA | −1.6 | −27.7 | 77 | −23.5 | −2.6 | −8.2 |
| | SEQ.ID.NO:506 | | | | | | |
| 110 | ACTGGAAGTCAGACTCATGG | −1.5 | −22.5 | 67.2 | −19.5 | −1.4 | −7.3 |
| | SEQ.ID.NO:507 | | | | | | |
| 168 | TTGCAGCTGTCCCAGCAGCA | −1.5 | −30.6 | 84.8 | −24.4 | 4.7 | −12.6 |
| | SEQ.ID.NO:508 | | | | | | |
| 236 | GTGAAGGTTTATCATAGCTT | −1.5 | −21 | 64.8 | −18.9 | 0 | −8.5 |
| | SEQ.ID.NO:509 | | | | | | |
| 140 | TGCTGCGATCCATTCAACTC | −1.4 | −25 | 70.5 | −23.6 | 0 | −6.4 |
| | SEQ.ID.NO:510 | | | | | | |
| 341 | ACAGAATGGGAACTTTTTGG | −1.3 | −19.8 | 59.6 | −17.8 | −0.4 | −3.8 |
| | SEQ.ID.NO:511 | | | | | | |
| 466 | TCAGGTAACTTCACGACAAG | −1.3 | −20.6 | 61.3 | −18.4 | −0.8 | −5 |
| | SEQ.ID.NO:512 | | | | | | |
| 555 | AAATGTGAAAGCTGCAGTTT | −1.2 | −19.6 | 59.2 | −17.5 | −0.2 | −9.6 |
| | SEQ.ID.NO:513 | | | | | | |
| 11 | GCGGCGTACTAAAGCACCGA | −1.1 | −26.8 | 70.2 | −24.1 | −1.5 | −5.9 |
| | SEQ.ID.NO:514 | | | | | | |
| 81 | TTGCAAAGGCGTGTGCACTA | −1.1 | −24.7 | 69.5 | −21.4 | −2 | −12 |
| | SEQ.ID.NO:515 | | | | | | |
| 144 | TAACTGCTGCGATCCATTCA | −1 | −24.3 | 68.4 | −23.3 | 0 | −5.7 |
| | SEQ.ID.NO:516 | | | | | | |
| 195 | AAAATCTTTTGTAAGCTAGA | −1 | −16.7 | 53.7 | −15.7 | 0 | −5.1 |
| | SEQ.ID.NO:517 | | | | | | |
| 329 | CTTTTTGGACCTCCAACAAC | −1 | −23 | 65.5 | −20.5 | −1.4 | −8.5 |
| | SEQ.ID.NO:518 | | | | | | |
| 398 | GATCAGAGGGCCCACATTGT | −1 | −27.4 | 76.8 | −24.8 | 0. | −11.3 |
| | SEQ.ID.NO:519 | | | | | | |
| 408 | TTTTCTTGATGATCAGAGGG | −1 | −21 | 64.5 | −20 | 0 | −6.8 |
| | SEQ.ID.NO:520 | | | | | | |
| 432 | CAAAAGTGTCCATTTAAGTT | −1 | −18.6 | 57.3 | −17.6 | 0 | −2.6 |
| | SEQ.ID.NO:521 | | | | | | |
| 453 | CGACAAGCTGATTTGCAGCA | −1 | −24.1 | 67.9 | −18.9 | −4.2 | −9.5 |
| | SEQ.ID.NO:522 | | | | | | |
| 177 | GATAACCAATTGCAGCTGTC | −0.9 | −22.7 | 66.1 | −20.9 | 0 | −9.7 |
| | SEQ.ID.NO:523 | | | | | | |
| 337 | AATGGGAACTTTTTGGACCT | −0.9 | −22 | 63.7 | −20.6 | −0.2 | −3.5 |
| | SEQ.ID.NO:524 | | | | | | |
| 355 | GTGTGAGCCCCATCACAGAA | −0.9 | −27.4 | 75.6 | −23.7 | −2.8 | −8.3 |
| | SEQ.ID.NO:525 | | | | | | |
| 460 | AACTTCACGACAAGCTGATT | −0.9 | −20.8 | 61 | −19.9 | 0 | −5.1 |
| | SEQ.ID.NO:526 | | | | | | |
| 8 | GCGTACTAAAGCACCGACTC | −0.8 | −24.5 | 67.7 | −23.2 | −0.1 | −4.3 |
| | SEQ.ID.NO:527 | | | | | | |
| 142 | ACTGCTGCGATCCATTCAAC | −0.8 | −24.8 | 69.5 | −24 | 0 | −6.4 |
| | SEQ.ID.NO:528 | | | | | | |
| 304 | TACACAGCTTTATCTCCCAA | −0.8 | −23.8 | 68.5 | −23 | 0 | −4.3 |
| | SEQ.ID.NO:529 | | | | | | |
| 342 | CACAGAATGGGAACTTTTTG | −0.8 | −19.3 | 58.4 | −17.8 | −0.4 | −3.4 |
| | SEQ.ID.NO:530 | | | | | | |
| 449 | AAGCTGATTTGCAGCATCAA | −0.8 | −22.2 | 65.1 | −17.2 | −4.2 | −9 |
| | SEQ.ID.NO:531 | | | | | | |
| 93 | TGGCGCCGTCGCTTGCAAAG | −0.7 | −28.7 | 74.2 | −24.7 | −3.3 | −13.3 |
| | SEQ.ID.NO:532 | | | | | | |
| 167 | TGCAGCTGTCCCAGCAGCAA | −0.7 | −29.8 | 81.7 | −24.4 | −4.7 | −12.6 |
| | SEQ.ID.NO:533 | | | | | | |
| 95 | CATGGCGCCGTCGCTTGCAA | −0.6 | −30.1 | 77.1 | −26.2 | −3.3 | −13.3 |
| | SEQ.ID.NO:534 | | | | | | |
| 435 | CATCAAAAGTGTCCATTTAA | −0.6 | −18.4 | 56.4 | −17.8 | 0 | −2.4 |
| | SEQ.ID.NO:535 | | | | | | |
| 87 | CGTCGCTTGCAAAGGCGTGT | −0.5 | −27.3 | 73.2 | −23.2 | −3.6 | −12.2 |
| | SEQ.ID.NO:536 | | | | | | |
| 448 | AGCTGATTTGCAGCATCAAA | −0.5 | −22.2 | 65.1 | −17.5 | −4.2 | −9 |
| | SEQ.ID.NO:537 | | | | | | |
| 184 | TAAGCTAGATAACCAATTGC | −0.4 | −19.1 | 57.8 | −18.7 | 0 | −6.2 |
| | SEQ.ID.NO:538 | | | | | | |
| 431 | AAAAGTGTCCATTTAAGTTT | −0.4 | −18 | 56.4 | −17.6 | 0 | −2.6 |
| | SEQ.ID.NO:539 | | | | | | |
| 578 | TGTTTCAACAAGACAAATGC | −0.4 | −17.9 | 55.3 | −17.5 | 0 | −5 |
| | SEQ.ID.140:540 | | | | | | |
| 579 | ATGTTTCAACAAGACAAATG | −0.4 | −16.1 | 51.5 | −15.2 | −0.2 | −5.9 |
| | SEQ.ID.NO:541 | | | | | | |
| 248 | GTCTTTCTGGATGTGAAGGT | −0.3 | −23.3 | 70.8 | −23 | 0 | −3.4 |
| | SEQ.ID.NO:542 | | | | | | |
| 94 | ATGGCGCCGTCGCTTGCAAA | −0.2 | −28.7 | 73.9 | −25.2 | −3.3 | −13.3 |
| | SEQ.ID.NO:543 | | | | | | |
| 350 | AGCCCCATCACAGAATGGGA | −0.2 | −27.4 | 74.1 | −24 | −3.2 | −9.4 |
| | SEQ.ID.NO:544 | | | | | | |
| 406 | TTCTTGATGATCAGAGGGCC | −0.2 | −24.6 | 72.1 | −23.7 | 0 | −9 |
| | SEQ.ID.NO:545 | | | | | | |
| 465 | CAGGTAACTTCACGACAAGC | −0.2 | −22 | 63.9 | −20.9 | −0.8 | −5 |
| | SEQ.ID.NO:546 | | | | | | |
| 173 | ACCAATTGCAGCTGTCCCAG | −0.1 | −27.8 | 76.1 | −27 | −0.1 | −9 |
| | SEQ.ID.NO:547 | | | | | | |
| 330 | ACTTTTTGGACCTCCAACAA | −0.1 | −23 | 65.5 | −21.7 | −1.1 | −8.2 |
| | SEQ.ID.NO:548 | | | | | | |
| 354 | TGTGAGCCCCATCACAGAAT | −0.1 | −26.2 | 72.3 | −23.7 | −2.4 | −7.7 |
| | SEQ.ID.NO:549 | | | | | | |
| 447 | GCTGATTTGCAGCATCAAAA | −0.1 | −21.5 | 62.8 | −18 | −3.4 | −8.3 |
| | SEQ.ID.NO:550 | | | | | | |
| 143 | AACTGCTGCGATCCATTCAA | 0 | −23.9 | 66.9 | −23.9 | 0 | −6.4 |
| | SEQ.ID.NO:551 | | | | | | |
| 556 | TAAATGTGAAAGCTGCAGTT | 0 | −19.2 | 58.4 | −18.6 | 0.5 | −8.9 |
| | SEQ.ID.NO:552 | | | | | | |
| 141 | CTGCTGCGATCCATTCAACT | 0.2 | −25.5 | 70.8 | −25.7 | 0 | −6.4 |
| | SEQ.ID.NO:553 | | | | | | |
| 407 | TTTCTTGATGATCAGAGGGC | 0.2 | −22.7 | 68.6 | −22.9 | 0 | −6.8 |
| | SEQ.ID.NO:554 | | | | | | |
| 164 | AGCTGTCCCAGCAGCAATGG | 0.3 | −28.5 | 78.8 | −24.5 | −4.3 | −10.4 |
| | SEQ.ID.NO:555 | | | | | | |
| 433 | TCAAAAGTGTCCATTTAAGT | 0.3 | −18.9 | 58.3 | −19.2 | 0 | −2.6 |
| | SEQ.ID.NO:556 | | | | | | |
| 451 | ACAAGCTGATTTGCAGCATC | 0.3 | −23.1 | 67.9 | −19.2 | −4.2 | −9 |
| | SEQ.ID.NO:557 | | | | | | |
| 12 | AGCGGCGTACTAAAGCACCG | 0.4 | −26.2 | 69.3 | −25.4 | −1.1 | −6.6 |
| | SEQ.ID.NO:558 | | | | | | |
| 85 | TCGCTTGCAAAGGCGTGTGC | 0.4 | −27.1 | 74.1 | −23.9 | −3.6 | −12.2 |
| | SEQ.ID.NO:559 | | | | | | |
| 179 | TAGATAACCAATTGCAGCTG | 0.4 | −20.8 | 61.3 | −20.4 | 0 | −9.2 |
| | SEQ.ID.NO:560 | | | | | | |
| 180 | CTAGATAACCAATTGCAGCT | 0.4 | −21.7 | 63.2 | −22.1 | 0 | −6.2 |
| | SEQ.ID.NO:561 | | | | | | |
| 239 | GATGTGAAGGTTTATCATAG | 0.4 | −18.8 | 59.3 | −19.2 | 0 | −4.6 |
| | SEQ.ID.NO:562 | | | | | | |
| 338 | GAATGGGAACTTTTTGGACC | 0.4 | −21.7 | 63.2 | −22.1 | 0.6 | −2.9 |
| | SEQ.ID.NO:563 | | | | | | |
| 434 | ATCAAAAGTGTCCATTTAAG | 0.5 | −17.7 | 55.4 | −18.2 | 0 | −2.6 |
| | SEQ.ID.NO:564 | | | | | | |
| 16 | TGCCAGCGGCGTACTAAAGC | 0.6 | −27 | 72.4 | −25.2 | −2.4 | −10.6 |
| | SEQ.ID.NO:565 | | | | | | |
| 454 | ACGACAAGCTGATTTGCAGC | 0.6 | −23.6 | 67.3 | −21.1 | −3.1 | −9.7 |
| | SEQ.ID.NO:566 | | | | | | |
| 464 | AGGTAACTTCACGACAAGCT | 0.7 | −22.2 | 64.6 | −22 | −0.7 | −6.1 |
| | SEQ.ID.NO:567 | | | | | | |
| 13 | CAGCGGCGTACTAAAGCACC | 0.8 | −26.1 | 70.2 | −26.1 | −0.6 | −6.6 |
| | SEQ.ID.NO:568 | | | | | | |
| 15 | GCCAGCGGCGTACTAAAGCA | 0.8 | −27.7 | 73.6 | −26.8 | −1.7 | −10 |
| | SEQ.ID.NO:569 | | | | | | |
| 194 | AAATCTTTTGTAAGCTAGAT | 0.8 | −17.4 | 55.5 | −18.2 | 0 | −5.4 |
| | SEQ.ID.NO:570 | | | | | | |
| 193 | AATCTTTTGTAAGCTAGATA | 0.9 | −17.8 | 56.9 | −18.2 | −0.1 | −5.7 |
| | SEQ.ID.NO:571 | | | | | | |
| 347 | CCCATCACAGAATGGGAACT | 0.9 | −24 | 66.7 | −22.2 | −2.7 | −8.2 |
| | SEQ.ID.NO:572 | | | | | | |
| 349 | GCCCCATCACAGAATGGGAA | 1.2 | −26.7 | 71.6 | −24.7 | −3.2 | −9.4 |
| | SEQ.ID.NO:573 | | | | | | |
| 192 | ATCTTTTGTAAGCTAGATAA | 1.3 | −17.8 | 56.9 | −19.1 | 0 | −5.1 |
| | SEQ.ID.NO:574 | | | | | | |
| 351 | GAGCCCCATCACAGAATGGG | 1.3 | −27.4 | 74.1 | −25.9 | −2.8 | −9.4 |
| | SEQ.ID.NO:575 | | | | | | |
| 172 | CCAATTGCAGCTGTCCCAGC | 1.7 | −29.4 | 79.8 | −28.7 | −2.4 | −11 |
| | SEQ.ID.NO:576 | | | | | | |
| 557 | ATAAATGTGAAAGCTGCAGT | 1.7 | −19.1 | 58 | −20.1 | −0.2 | −8.7 |
| | SEQ.ID.NO:577 | | | | | | |
| 569 | AAGACAAATGCCATAAATGT | 1.8 | −17.4 | 53.2 | −19.2 | 0 | −3.3 |
| | SEQ.ID.NO:578 | | | | | | |
| 183 | AAGCTAGATAACCAATTGCA | 1.9 | −20.1 | 59.5 | −21.4 | −0.3 | −6.2 |
| | SEQ.ID.NO:579 | | | | | | |
| 357 | TTGTGTGAGCCCCATCACAG | 1.9 | −27.6 | 76.9 | −26.7 | −2.8 | −8.3 |
| | SEQ.ID.NO:580 | | | | | | |
| 356 | TGTGTGAGCCCCATCACAGA | 2.3 | −28.1 | 77.9 | −27.6 | −2.8 | −8.3 |
| | SEQ.ID.NO:581 | | | | | | |
| 171 | CAATTGCAGCTGTCCCAGCA | 2.4 | −28.1 | 77.4 | −27 | −3.5 | −11.4 |
| | SEQ.ID.NO:582 | | | | | | |
| 576 | TTTCAACAAGACAAATGCCA | 2.4 | −19.4 | 57.4 | −21.8 | 0 | −3 |
| | SEQ.ID.NO:583 | | | | | | |
| 165 | CAGCTGTCCCAGCAGCAATG | 2.5 | −28 | 77.3 | −26.2 | −4.3 | −9.6 |
| | SEQ.ID.NO:584 | | | | | | |
| 575 | TTCAACAAGACAAATGCCAT | 2.5 | −19.3 | 57.1 | −21.8 | 0 | −3 |
| | SEQ.ID.NO:585 | | | | | | |
| 399 | TGATCAGAGGGCCCACATTG | 2.6 | −26.2 | 73.2 | −27.2 | 0 | −11.3 |
| | SEQ.ID.NO:586 | | | | | | |
| 14 | CCAGCGGCGTACTAAAGCAC | 2.7 | −26.1 | 70.2 | −27.8 | −0.9 | −6.6 |
| | SEQ.ID.NO:587 | | | | | | |
| 348 | CCCCATCACAGAATGGGAAC | 2.8 | −25.1 | 68.3 | −24.7 | −3.2 | −9 |
| | SEQ.ID.NO:588 | | | | | | |
| 352 | TGAGCCCCATCACAGAATGG | 3 | −26.2 | 71.6 | −28.1 | −1 | −5.2 |
| | SEQ.ID.NO:589 | | | | | | |
| 170 | AATTGCAGCTGTCCCAGCAG | 3.1 | −27.4 | 76.6 | −27 | −3.5 | −11.2 |
| | SEQ.ID.NO:590 | | | | | | |
| 191 | TCTTTTGTAAGCTAGATAAC | 3.1 | −18 | 57.5 | −21.1 | 0 | −5.1 |
| | SEQ.ID.NO:591 | | | | | | |
| 238 | ATGTGAAGGTTTATCATAGC | 3.2 | −20 | 62.3 | −23.2 | 0 | −5.7 |
| | SEQ.ID.NO:592 | | | | | | |
| 577 | GTTTCAACAAGACAAATGCC | 3.4 | −19.9 | 59 | −23.3 | 0 | −3.7 |
| | SEQ.ID.NO:593 | | | | | | |
| 565 | CAAATGCCATAAATGTGAAA | 3.7 | −16.5 | 51.1 | −20.2 | 0 | −3.3 |
| | SEQ.ID.NO:594 | | | | | | |
| 566 | ACAAATGCCATAAATGTGAA | 3.7 | −17.4 | 53.1 | −21.1 | 0 | −3.3 |
| | SEQ.ID.NO:595 | | | | | | |
| 574 | TCAACAAGACAAATGCCATA | 3.7 | −18.9 | 56.3 | −22.6 | 0 | −3 |
| | SEQ.ID.NO:596 | | | | | | |
| 86 | GTCGCTTGCAAAGGCGTGTG | 3.8 | −26.5 | 73.2 | −26.7 | −3.6 | −12.2 |
| | SEQ.ID.NO:597 | | | | | | |
| 558 | CATAAATGTGAAAGCTGCAG | 3.9 | −18.6 | 56.4 | −21.9 | −0.2 | −8.2 |
| | SEQ.ID.NO:598 | | | | | | |
| 573 | CAACAAGACAAATGCCATAA | 3.9 | −17.8 | 53.5 | −21.7 | 0 | −3 |
| | SEQ.ID.NO:599 | | | | | | |
| 564 | AAATGCCATAAATGTGAAAG | 4.3 | −15.8 | 50 | −20.1 | 0 | −3.3 |
| | SEQ.ID.NO:600 | | | | | | |
| 568 | AGACAAATGCCATAAATGTG | 4.3 | −18.1 | 54.9 | −22.4 | 0 | −3.4 |
| | SEQ.ID.NO:601 | | | | | | |
| 405 | TCTTGATGATCAGAGGGCCC | 4.4 | −26.5 | 75.4 | −29.9 | 0 | −10 |
| | SEQ.ID.NO:602 | | | | | | |
| 166 | GCAGCTGTCCCAGCAGCAAT | 4.7 | −29.8 | 81.9 | −30.2 | −4.3 | −12 |
| | SEQ.ID.NO:603 | | | | | | |
| 559 | CCATAAATGTGAAAGCTGCA | 5.1 | −20.6 | 59.8 | −25.2 | −0.2 | −6.5 |
| | SEQ.ID.NO:604 | | | | | | |
| 563 | AATGCCATAAATGTGAAAGC | 5.2 | −18.3 | 55.1 | −23.5 | 0 | −3.1 |
| | SEQ.ID.NO:605 | | | | | | |
| 404 | CTTGATGATCAGAGGGCCCA | 5.3 | −26.8 | 74.8 | −30.5 | 0 | 11.3 |
| | SEQ.ID.NO:606 | | | | | | |
| 570 | CAAGACAAATGCCATAAATG | 5.5 | −16.9 | 51.9 | −22.4 | 0 | −3 |
| | SEQ.ID.NO:607 | | | | | | |
| 403 | TTGATGATCAGAGGGCCCAC | 5.7 | −26.1 | 73.4 | −30.2 | 0 | −11.3 |
| | SEQ.ID.NO:608 | | | | | | |
| 562 | ATGCCATAAATGTGAAAGCT | 5.7 | −19.9 | 58.6 | −25.1 | −0.2 | −4.8 |
| | SEQ.ID.NO:609 | | | | | | |
| 571 | ACAAGACAAATGCCATAAAT | 5.9 | −17.1 | 52.4 | −23 | 0 | −3 |
| | SEQ.ID.NO:610 | | | | | | |
| 237 | TGTGAAGGTTTATCATAGCT | 6.3 | −20.9 | 64.3 | −27.2 | 0 | −7.1 |
| | SEQ.ID.NO:611 | | | | | | |
| 572 | AACAAGACAAATGCCATAAA | 6.3 | −16.4 | 50.8 | −22.7 | 0 | −3 |
| | SEQ.ID.NO:612 | | | | | | |
| 353 | GTGAGCCCCATCACAGAATG | 6.5 | −26.2 | 72.3 | −31 | −1.7 | −6.4 |
| | SEQ.ID.NO:613 | | | | | | |
| 561 | TGCCATAAATGTGAAAGCTG | 7.5 | −19.9 | 58.6 | −26.9 | −0.2 | −5.1 |
| | SEQ.ID.NO:614 | | | | | | |
| 181 | GCTAGATAACCAATTGCAGC | 7.9 | −22.6 | 65.4 | −30.5 | 0 | −6.2 |
| | SEQ.ID.NO:615 | | | | | | |
| 182 | AGCTAGATAACCAATTGCAG | 9 | −20.8 | 61.6 | −29.2 | −0.3 | −6.2 |
| | SEQ.ID.NO:616 | | | | | | |
| 560 | GCCATAAATGTGAAAGCTGC | 9.6 | −21.7 | 62.4 | −30.8 | −0.2 | −5.2 |
| | SEQ.ID.NO:617 | | | | | | |

### Example 15

Western Blot Analysis of mitoNEET Protein Levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to mitoNEET is used, with a radiolabelled or fluorescently labeled secondary antibody directed against the primary antibody species. Bands are visualized using a PHOSPHORIMAGER™ (Molecular Dynamics, Sunnyvale Calif.).

What is claimed is:

1. An antisense compound 8 to 30 nucleobases in length targeted to a nucleic acid molecule encoding mitoNEET, wherein said antisense compound specifically hybridizes with and inhibits the expression of mitoNEET.

2. The antisense compound of claim 1 which is an antisense oligonucleotide.

3. The antisense compound of claim 2 wherein said antisense oligonucleotide comprises at least 8 contiguous nucleic acids of a nucleic acid sequence of SEQ ID NO.1-SEQ ID NO:617.

4. The antisense compound of claim 3 wherein said antisense oligonucleotide comprises a nucleic acid sequence of SEQ ID NO. 1-SEQ ID NO:617.

5. The antisense compound of claim 2 wherein said antisense oligonucleotide consists of at least 8 contiguous nucleic acids of a nucleic acid sequence of SEQ ID NO. 1-SEQ ID NO:617.

6. The antisense compound of claim 2 wherein said antisense oligonucleotide consists of a nucleic acid sequence of SEQ ID NO. 1-SEQ ID NO:617.

7. The antisense compound of claim 1 wherein the antisense oligonucleotide comprises at least one modified internucleoside linkage.

8. The antisense compound of claim 1 or 7 wherein the antisense oligonucleotide comprises at least one modified sugar moiety.

9. The antisense compound of claim 1 or 7 wherein the antisense oligonucleotide comprises at least one modified nucleobase.

10. The antisense compound of claim 8 wherein the antisense oligonucleotide comprises at least one modified nucleobase.

11. A composition comprising the antisense compound of claim 1 and a pharmaceutically acceptable carrier or diluent.

12. A composition comprising the antisense compound of claim 7 and a pharmaceutically acceptable carrier or diluent.

13. A composition comprising the antisense compound of claim 8 and a pharmaceutically acceptable carrier or diluent.

14. A composition comprising the antisense compound of claim 9 and a pharmaceutically acceptable carrier or diluent.

15. A composition comprising the antisense compound of claim 10 and a pharmaceutically acceptable carrier or diluent.

16. A method of treating a human having a disease or condition associated with mitoNEET comprising administering to said animal a therapeutically or prophylactically effective amount of the antisense compound of claim 1 so that expression of mitoNEET is inhibited.

17. The method of claim 16 wherein the disease or condition is selected from diabetes, an immunological disorder, a cardiovascular disorder including hypertension, a neurologic disorder, and ischemia/reperfusion injury.

18. A method of treating a human having a disease or condition associated with mitoNEET comprising administering to said animal a therapeutically or prophylactically effective amount of the antisense compound of claim 7 so that expression of mitoNEET is inhibited.

19. The method of claim 18 wherein the disease or condition is selected from diabetes, an immunological disorder, a cardiovascular disorder including hypertension, a neurologic disorder, and ischemia/reperfusion injury.

20. A method of treating a human having a disease or condition associated with mitoNEET comprising administering to said animal a therapeutically or prophylactically effective amount of the antisense compound of claim 8 so that expression of mitoNEET is inhibited.

21. The method of claim 20 wherein the disease or condition is selected from diabetes, an immunological disorder, a cardiovascular disorder including hypertension, a neurologic disorder, and ischemia/reperfusion injury.

22. A method of treating a human having a disease or condition associated with mitoNEET comprising administering to said animal a therapeutically or prophylactically effective amount of the antisense compound of claim 9 so that expression of mitoNEET is inhibited.

23. The method of claim 22 wherein the disease or condition is selected from diabetes, an immunological disorder, a cardiovascular disorder including hypertension, a neurologic disorder, and ischemia/reperfusion injury.

24. A method of treating a human having a disease or condition associated with mitoNEET comprising administering to said animal a therapeutically or prophylactically effective amount of the antisense compound of claim 10 so that expression of mitoNEET is inhibited.

25. The method of claim 24 wherein the disease or condition is selected from diabetes, an immunological disorder, a cardiovascular disorder including hypertension, a neurologic disorder, and ischemia/reperfusion injury.
